(19) United States
(12) Patent Application Publication
(10) Pub. No.: US 2025/0052755 A1
(43) Pub. Date: Feb. 13, 2025

(54) FLUORESCENT BIOSENSOR FOR ACETYL COENZYME A

(71) Applicant: UNIVERSITY OF UTAH RESEARCH FOUNDATION, Salt Lake City, UT (US)

(72) Inventors: Katharine L. Diehl, Salt Lake City, UT (US); Joseph J. Smith, Salt Lake City, UT (US)

(21) Appl. No.: 18/448,632

(22) Filed: Aug. 11, 2023

(51) Int. Cl. G01N 33/573; C07K 14/00

(52) U.S. Cl. CPC G01N 33/5735; C07K 14/001; C07K 2319/60

(57) ABSTRACT

Disclosed herein are a polypeptide biosensor and compositions comprising the polypeptide biosensor that detects acetyl coenzyme A (acetyl-CoA). The polypeptide comprises an acetyl-CoA binding protein and a fluorescent protein. Further described herein are methods of using the biosensor to detect acetyl-CoA and expression vectors comprising the biosensor.

### STATEMENT REGARDING FEDERALLY SPONSORED RESEARCH

This invention was made with government support under grant R35GM143080 awarded by the National Institutes of Health. The government has certain rights in the invention.

### REFERENCE TO AN ELECTRONIC SEQUENCE LISTING

This application was filed with a Sequence Listing XML in ST.26 XML format in accordance with 37 C.F.R. § 1.821. The Sequence Listing XML file submitted in the USPTO Patent Center, “026389-0021-US01.xml,” was created on Aug. 11, 2023, contains 88 sequences, has a file size of 134 Kbytes, and is incorporated by reference in its entirety into the specification.

### FIELD

This disclosure relates to a polypeptide biosensor and compositions comprising the polypeptide biosensor that detects acetyl coenzyme A (acetyl-CoA). The polypeptide comprises an acetyl-CoA binding protein and a fluorescent protein. The disclosure further relates to methods of using the biosensor to detect acetyl-CoA and expression vectors comprising the biosensor.

### INTRODUCTION

Acetyl-coenzyme A (acetyl-CoA) is a core metabolite that serves central metabolic, catabolic, and signaling functions. Current methods to quantify acetyl-CoA from cells include enzyme-coupled assays such as PicoProbe™ Acetyl-CoA assay (BioVision, Milpitas, CA) and mass spectrometry. Due to the relatively low abundance and stability of acetyl-CoA and other short-chain acyl-CoAs, indirect methods of acetyl-CoA detection are commonly employed. However, these methods are inherently destructive and require fractionation to obtain subcellular resolution. Fluorescent biosensors have been developed for cellular metabolites to enable real time imaging of, for example, ATP, NAD+, and glucose in live cells. However, no such biosensor exists for acetyl-CoA.

Thus, there is a need for a biosensor that detects acetyl-CoA.

### SUMMARY

In an aspect, the disclosure relates to a recombinant acetyl-coenzyme A (acetyl-CoA) biosensor polypeptide that may comprise an acetyl-CoA binding protein having an amino acid sequence at least 95% identical to SEQ ID NO: 1, wherein the acetyl-CoA binding protein may be divided into: a first acetyl-CoA binding protein fragment comprising an N-terminal portion of the acetyl-CoA binding protein; and a second acetyl-CoA binding protein fragment comprising a C-terminal portion of the acetyl-CoA binding protein, wherein the first and second acetyl-CoA binding protein fragments collectively may include all of the amino acids of the acetyl-CoA binding protein; and a fluorescent protein may be inserted between the first and second acetyl-CoA binding protein fragments and attached to a C-terminus of the first acetyl-CoA binding protein fragment and an N-terminus of the second acetyl-CoA binding protein fragment; and wherein: (i) the C-terminus may be an arginine at position 69 of SEQ ID NO: 1 (Arg69) and the N-terminus may be a glutamic acid at position 70 of SEQ ID NO: 1 (Glu70); (ii) the C-terminus may be a tryptophan at position 23 of SEQ ID NO: 1 (Trp23) and the N-terminus may be a proline at position 24 of SEQ ID NO: 1 (Pro24); (iii) the C-terminus may be a valine at position 71 of SEQ ID NO: 1 (Val71) and the N-terminus may be a threonine at position 72 of SEQ ID NO: 1 (Thr72); (iv) the C-terminus may be an aspartic acid at position 99 of SEQ ID NO: 1 (Asp99) and the N-terminus may be an alanine at position 100 of SEQ ID NO: 1 (Ala100); (v) the C-terminus may be an aspartic acid at 104 of SEQ ID NO: 1 (Asp104) and the N-terminus may be an arginine at position 105 of SEQ ID NO: 1 (Arg105); or (vi) the C-terminus may be a glycine at position 116 of SEQ ID NO: 1 (Glyl16) and the N-terminus may be a phenylalanine at position 117 of SEQ ID NO: 1 (Phe117); and wherein the recombinant acetyl-CoA biosensor polypeptide may selectively bind acetyl-CoA, and the binding of acetyl-CoA may induce a change in the fluorescence of the fluorescent protein. In an embodiment, the fluorescent protein may be a circularly permuted GFP (cpGFP), a circularly permuted yellow fluorescent protein (cpYFP), or a circularly permuted blue fluorescent protein (cpBFP). In some embodiments, the cpGFP may comprise an amino acid sequence of SEQ ID NO: 2, the cpYFP may comprise an amino acid sequence of SEQ ID NO: 3, and the cpBFP may comprise an amino acid sequence of SEQ ID NO: 4. In some embodiments, the acetyl-CoA binding protein may comprise an amino acid sequence at least 99% identical to SEQ ID NO: 1. In some embodiments, the acetyl-CoA binding protein may comprise the amino acid sequence of SEQ ID NO: 1. In some embodiments, the fluorescent protein may be either directly attached to the C-terminus of the first acetyl-CoA binding protein fragment or may be attached by a first amino acid linker that is from 1 to 3 amino acids in length; and the fluorescent protein may be either directly attached to the N-terminus of the second acetyl-CoA binding protein fragment or may be attached by a second linker that is from 1 to 3 amino acids in length. In some embodiments, the first and second amino acid linkers may be each independently selected from the group consisting of a Gly, Gly-Ala, Ala-Ser, and Gly-Ala-Ser. In some embodiments, (i) the first linker may be Gly-Ala and the second linker may be Gly-Ala; (ii) the first linker may be Ala-Ser and the second linker may be Ala-Ser; (iii) the first linker may be Gly-Ala-Ser and the second linker may be Gly; (iv) the C-terminus and N-terminus are directly attached to the fluorescent protein; (v) the C-terminus may be directly attached to the fluorescent protein and the second linker may be Gly-Ala-Ser; (vi) the first linker may be Gly-Ala-Ser and the N-terminus may be directly attached to the fluorescent protein; (vii) the first linker may be Gly-Ala and the N-terminus may be directly attached to the fluorescent protein; or (viii) the first linker may be Gly and the second linker may be Gly-Ala-Ser. In some embodiments, the recombinant acetyl-CoA biosensor polypeptide may further comprise one or more of a histidine tag, a TEV cleavage site, a FLAG® tag, a human influenza hemagglutinin (HA) tag, a nuclear export signal, a nuclear localization signal, a cytoplasmic localization signal, and a mitochondrial localization signal at the N-terminal portion of the acetyl-CoA binding protein. In some embodiments, (i) the recombinant acetyl-CoA biosensor polypeptide may comprise the amino acid sequence of SEQ ID NO: 5; (ii) the recombinant acetyl-CoA biosensor polypeptide may comprise the amino acid sequence of SEQ ID NO: 6; (iii) the recombinant acetyl-CoA biosensor polypeptide may comprise the amino acid sequence of SEQ ID NO: 7; (iv) the recombinant acetyl-CoA biosensor polypeptide may comprise the amino acid sequence of SEQ ID NO: 8; (v) the recombinant acetyl-CoA biosensor polypeptide may comprise the amino acid sequence of SEQ ID NO: 9; (vi) the recombinant acetyl-CoA biosensor polypeptide may comprise the amino acid sequence of SEQ ID NO: 10; (vii) the recombinant acetyl-CoA biosensor polypeptide may comprise the amino acid sequence of SEQ ID NO: 11; (viii) the recombinant acetyl-CoA biosensor polypeptide may comprise the amino acid sequence of SEQ ID NO: 12; (ix) the recombinant acetyl-CoA biosensor polypeptide may comprise the amino acid sequence of SEQ ID NO: 13; (x) the recombinant acetyl-CoA biosensor polypeptide may comprise the amino acid sequence of SEQ ID NO: 14; (xi) the recombinant acetyl-CoA biosensor polypeptide may comprise the amino acid sequence of SEQ ID NO: 15; (xii) the recombinant acetyl-CoA biosensor polypeptide may comprise the amino acid sequence of SEQ ID NO: 16; (xiii) the recombinant acetyl-CoA biosensor polypeptide may comprise the amino acid sequence of SEQ ID NO: 17; (xiv) the recombinant acetyl-CoA biosensor polypeptide may comprise the amino acid sequence of SEQ ID NO: 18; or (xv) the recombinant acetyl-CoA biosensor polypeptide may comprise the amino acid sequence of SEQ ID NO: 19. In some embodiments, the recombinant acetyl-CoA biosensor polypeptide may comprise the amino acid of SEQ ID NO: 5.

In a further aspect, the disclosure relates to an expression vector comprising: a nucleic acid that encodes the recombinant acetyl-CoA biosensor polypeptide as descried herein; and a promoter operably linked to the nucleic acid. In an embodiment, the expression vector may be a lentiviral vector, an adeno-associated virus (AAV) vector, or a cytomegalovirus (CMV) vector.

Another aspect of the disclosure provides a method of detecting acetyl-CoA in a sample that may comprise contacting the sample with the recombinant acetyl-CoA biosensor polypeptide as described herein; exciting the recombinant acetyl-CoA biosensor polypeptide in the sample at an excitation wavelength; measuring a fluorescence intensity of the recombinant acetyl-CoA biosensor polypeptide in the sample at an emission wavelength; and comparing the fluorescence intensity to a standard curve, wherein the fluorescence intensity correlates with a concentration of acetyl-CoA in the sample. In an embodiment, the excitation wavelength may be from about 460 nm to about 490 nm. In some embodiments, the excitation wavelength may be 485 nm. In some embodiments, the emission wavelength may be from about 513 nm to about 540 nm. In some embodiments, the emission wavelength may be 514 nm. In some embodiments, the pH of the sample may be maintained at a pH of 6.5-8.0.

Another aspect of the disclosure provides a method of monitoring acetyl-CoA activity in a cell, comprising: providing a cell with the recombinant acetyl-CoA biosensor polypeptide of any one of clauses 1-11; exciting the recombinant acetyl-CoA biosensor polypeptide in the cell at a first excitation wavelength between about 400 nm and about 430 nm while measuring a first fluorescence intensity at an emission wavelength between about 513 nm and about 540 nm; exciting the recombinant acetyl-CoA biosensor polypeptide in the cell at a second excitation wavelength between about 460 nm and about 490 nm while measuring a second fluorescence intensity at the emission wavelength; and normalizing the second fluorescence intensity based on the first fluorescence intensity. In some embodiments, normalizing may comprise dividing the second fluorescence intensity by the first fluorescence intensity. In some embodiments, the method may further comprise treating the cell with an acetyl-CoA precursor or nutrient affecting the function of the cell and comparing the normalized fluorescence intensity of the cell to the normalized fluorescence intensity of a control cell. In some embodiments, one or more of a nuclear export signal, a nuclear localization signal, a cytoplasmic localization signal, and a mitochondrial localization signal may be attached to an N-terminus of the recombinant acetyl-CoA biosensor polypeptide. In some embodiments, the method may further comprise determining where acetyl-CoA is localized in the cell. In some embodiments, the first excitation wavelength may be 405 nm. In some embodiments, the second excitation wavelength may be 485 nm. In some embodiments, the emission wavelength may be 514 nm. In some embodiments, the providing step may comprise transforming the cell with a plasmid comprising a polynucleotide that encodes the recombinant acetyl-CoA biosensor polypeptide.

The disclosure provides for other aspects and embodiments that will be apparent in light of the following detailed description and accompanying figures.

### BRIEF DESCRIPTION OF THE DRAWINGS

FIG. 1A is a graph showing a representative plot of surface plasmon resonance (SPR) data for acetyl coenzyme A (acetyl-CoA) binding to a PanZ-CFP fusion protein. The dissociation constants (“KD”) are shown in the figure. FIG. 1B is a graph showing a representative plot of surface plasmon resonance (SPR) data for coenzyme A (CoA) binding to a PanZ-CFP fusion protein. The dissociation constants (“KD”) are shown in the figure. FIG. 1C is a table of kinetic data for acetyl-CoA binding to a PanZ-CFP fusion protein obtained from the SPR experiments like those depicted in FIG. 1A. FIG. 1D is a table of kinetic data for acetyl-CoA binding to a PanZ-CFP fusion protein obtained from the SPR experiments like those depicted in FIG. 1B. This data shows that PanZ has selectivity for binding to acetyl-CoA over the structurally very similar CoA molecule, suggesting that it would have sufficient selectivity to be the basis for a useful biosensor in cells where both molecules exist.

FIG. 2 is a model depicting the structure of PanZ (PDB 4CRZ) with its six unstructured loops labeled that were tested as cpGFP insertion sites to make an acetyl-CoA biosensor described herein.

FIG. 3 is a bar graph showing the fluorescence intensity of a series of PanZ-cpGFP fusion proteins in the presence of 1 mM acetyl-CoA or in the absence of acetyl-CoA. λₑₓ=485 nm, λₑₘ=514 nm, n=3. All of these fusions contain an Ala-Ser (AS) linker at each junction, i.e., (Nterm) PanZ-AS-cpGFP-AS-PanZ (Cterm). The PanZ-cpGFP fusion proteins vary in their overall intensity depending on the site of cpGFP insertion. Some of these proteins, such as when the linker is attached to asparagine at position 45 and proline at position 90, have a very low fluorescence intensity, suggesting they would be poor sensors.

FIG. 4 is a bar graph showing the fluorescence change of the same series of PanZ-cpGFP fusion proteins from FIG. 3 in the presence of 1 mM acetyl-CoA compared to in the absence of acetyl-CoA ((F₁−F₀)/F₀). λₑₓ=485, λₑₘ=514 nm, n=3. The location of each cpGFP insertion site listed at the bottom of the graph is labeled according to what loop it corresponds with in FIG. 2. All of these fusions contain an Ala-Ser (AS) linker at each junction, i.e., (Nterm) PanZ-AS-cpGFP-AS-PanZ (Cterm). From this data, it is shown that several of the fusion proteins show a reproducible change in intensity in the presence of acetyl-CoA that is a larger change than for cpGFP alone, which represents the background since cpGFP has no intrinsic acetyl-CoA binding ability. These include tryptophan at position 23 (W23), arginine at position 69 (R69), valine at position 71 (V71), aspartic acid at position 99 (D99), aspartic acid at position 104 (D104), and glycine at position 116 (G116). R69 had the largest magnitude of change, so it was selected to further optimize. Nevertheless, the other foregoing insertion sites can be characterized as acetyl-CoA sensors as well.

FIG. 5 is a heat map showing the fluorescence response of a series of linker variations of the PanZ (R69)-N-termlinker-cpGFP-C-termlinker-(E70) PanZ fusion protein, where in this instance, the N-termlinker is the peptide linker that attaches the N-terminal end of the fluorescent protein cpGFP to the C terminal end of the PanZ (R69) acetyl CoA binding protein fragment, and the C-termlinker is the peptide linker that attaches the C-terminal end of the fluorescent protein to the N-terminal end of the PanZ (E70) acetyl-CoA binding protein fragment. The N-terminal linker was either Gly-Ala-Ser (GAS), Gly-Ala (GA), Gly (G), or no linker and was paired with a C-terminal linker of either Gly-Ala-Ser (GAS), Gly-Ala (GA), Gly (G), or no linker as shown on the axes of the heat map. The data was quantified as a fold-change in each fusion protein's fluorescence emission in the presence of 1 mM acetyl-CoA compared to in the absence of acetyl-CoA. (F₁−F₀)/F₀). λₑₓ=485, λₑₘ=514 nm. From this data, it is shown that the N-terminal GA (N-GA)/C-terminal GA (C-GA) linker combination gives the largest magnitude of sensor response (about 0.6-fold). However, the N-terminal GAS (N-GAS)/C-terminal G (C-G), no linker on the N-terminal (NO)/no linker on the C-terminal (CO), and no linker on the N-terminal (NO)/C-terminal GAS (C-GAS) linker combinations are also well above the background defined by cpGFP alone in FIGS. 3-4. N-GAS/CO, N-GA/CO, and N-terminal G (N-G)/C-GAS are also above that background level, performing similarly to the original N-terminal AS (N-AS)/C-terminal AS (C-AS) linker. The N-GA/C-GA linker was further studied because it had the highest fluorescence response when compared to the other linkers that were tested.

FIG. 6 is a bar graph depicting the fluorescence response of the PanZ (R69)-AS-cpGFP-AS-(E70) PanZ fusion protein from FIG. 4 and of the PanZ (R69)-GA-cpGFP-GA-(E70) PanZ fusion protein from FIG. 5 as a comparison. The N-GA/C-GA linker combination significantly enhanced the magnitude of the fluorescence change of the sensor compared to N-AS/C-AS linker combination. A big dynamic range equates to a better detection sensitivity, so the N-GA/C-GA linker combination was further studied.

FIG. 7 is a bar graph showing the fluorescence response of the PanZ (R69)-GA-cpGFP-GA-(E70) PanZ fusion protein from FIG. 4 compared to the same yellow and blue fluorescent protein fusions (i.e., PanZ (R69)-GA-cpYFP-GA-(E70) PanZ and PanZ (R69)-GA-cpBFP-GA-(E70) PanZ) to acetyl-CoA. ((F₁−F₀)/F₀). cpGFP: λₑₓ=485, λₑₘ=514 nm; cpYFP: λₑₓ=505, λₑₘ=535 nm; cpBFP: λₑₓ=389, λₑₘ=440 nm. The fluorescence response of the fusion protein comprising cpGFP had the largest dynamic range of the three fusion proteins, but the fluorescence response of the fusion protein comprising cpYFP was above background fluorescence. The fluorescence response of the fusion protein comprising cpBFP was similar to that of the fusion protein comprising cpGFP and the N-AS/C-AS linker combination shown in FIG. 6.

FIG. 8 is an image of a Coomassie stained SDS-PAGE gel of the purified PanZ (R69)-GA-cpGFP-GA-(E70) PanZ fusion protein (“sensor”; “elution” lane).

FIG. 9 is a line graph depicting the fluorescence response of the sensor (i.e., PanZ (R69)-GA-cpGFP-GA-(E70) PanZ fusion protein or “PancAce”) and of cpGFP alone to a range of concentrations of acetyl-CoA or CoA as indicated on the X-axis of the line graph. ((F₁-F₀)/F₀). λₑₓ=485, λₑₘ=514 nm, n=3-5. This line graph shows that the sensor responds only to acetyl-CoA and not CoA, which is consistent with FIGS. 1A-1D. This line graph also shows the range of concentrations of acetyl-CoA that the sensor can detect (from about 10 μM up to about 2 mM with a K_{D} of about 250 μM).

FIG. 10A and FIG. 10B are molecular models showing the acetyl-CoA binding sites of PanZ from Protein Data Bank (PDB) 4CRZ (FIG. 10A) and the sensor (i.e., PanZ (R69)-AS-cpGFP-AS-(E70) PanZ or “PancAce”) from molecular modeling (FIG. 10B). The schematics highlight the interactions between the amino acids in each protein with the acetyl-CoA molecule. This shows that the insertion of the cpGFP appears to disrupt the acetyl-CoA binding site in PanZ to an extent and explains why the affinity of the sensor for acetyl-CoA is lower than that of PanZ by itself.

FIG. 11 is a bar graph showing the fluorescence response of the sensor (i.e., PanZ (R69)-GA-cpGFP-GA-(E70) PanZ or “PancAce”) and of cpGFP alone to a 1 mM concentration of different biologically-relevant acyl-CoAs as indicated on the X-axis. (F₁−F₀)/F₀). λₑₓ=485, λₑₘ=514 nm, n=3. These data show how selective the sensor is for acetyl-CoA over other structurally similar molecules that would be expected to be in samples that would be used for detection of acetyl-CoA with the sensor. The sensor is highly selective for acetyl-CoA over most of the acyl-CoA species, but the sensor does have some response to propionyl-CoA, which varies from acetyl-CoA by only one CH₂ group.

FIG. 12 is a line graph showing the fluorescence response of the sensor (i.e., PanZ (R69)-GA-cpGFP-GA-(E70) PanZ or “PancAce”) and of cpGFP alone to a range of propionyl-CoA concentrations (F₁−F₀)/F₀). The data for acetyl-CoA and CoA are reproduced here from FIG. 9. The sensor has an approximately 2.5-fold higher affinity for acetyl-CoA compared to propionyl-CoA. λₑₓ=485, λₑₘ=514 nm, n=3-5. This graph quantifies the selectivity of the sensor for acetyl-CoA over propionyl-CoA since propionyl-CoA was the only potential interferent based on FIG. 11. The sensor prefers acetyl-CoA by a factor of about 2.5-fold. Based on the amount of propionyl-CoA in cells compared to acetyl-CoA, this margin should not affect the accuracy of the sensor for detecting acetyl-CoA.

FIG. 13 is a line graph depicting the fluorescence emission spectra of the sensor (i.e., PanZ (R69)-GA-cpGFP-GA-(E70) PanZ or “PancAce”) and of cpGFP alone collected using an excitation wavelength of 485 nm. The emission peak of the sensor is from 513-540 nm. 514 nm was used for other experiments herein because it is at the top of the emission spectra peak.

FIG. 14 is a line graph showing the fluorescence excitation spectra of the sensor (i.e., PanZ (R69)-GA-cpGFP-GA-(E70) PanZ or “PancAce”) and of cpGFP when incubated with concentrations of acetyl-CoA that are specified on the X-axis. The data was collected using an emission wavelength of 514 nm. There are two excitation peaks, one at 405 nm (400-430 nm) and one at 485 nm (460-490 nm). This figure shows the 405 nm and 485 nm excitation peaks of the sensor. It further shows that the 405 nm excitation is essentially invariant to the presence of acetyl-CoA, while the 485 nm peak is highly sensitive to the presence of acetyl-CoA. This shows that the 485 nm/405 nm enables ratiometric imaging, which can improve the accuracy of the sensor measurements, especially in cell imaging applications.

FIG. 15 is a dot plot showing the fluorescence intensity of the sensor (i.e., PanZ (R69)-GA-cpGFP-GA-(E70) PanZ or “PancAce”) and of cpGFP alone in response to a range of pHs (F₁−F₀)/F₀). λₑₓ=485, λₑₘ=514 nm, n=3. This figure shows that the sensor has a similar emission intensity dependence on pH (i.e., pH sensitivity) to cpGFP alone.

FIG. 16 is a dot plot showing the fluorescence of live E. coli that express either PancAce (sensor) or cpGFP and were deprived of all glucose over the time period shown on the X-axis of the plot. Depriving the E. coli of glucose in their media should cause their acetyl-CoA levels to drop over time because glucose is their main metabolic precursor of acetyl-CoA. The fluorescence (λₑₓ=405 nm, 485 nm; λₑₘ=514 nm) was measured by flow cytometry. The fluorescence was normalized by dividing F₄₈₈/F₄₀₅ and then (F₁−F₀)/F₀ where F₀ were non-deprived cells, n=3, ** p<0.005, *** p<0.0005.

FIG. 17 is a dot plot showing the fluorescence of live E. coli that express either PancAce (sensor) or cpGFP and were first deprived of all glucose for 3 hours and then refed with 28 mM glucose for the time periods shown on the X-axis of the plot. If E. coli were deprived of glucose for 3 h, it is known from FIG. 16 that their acetyl-CoA levels drop. Here, if the E. coli are refed, the acetyl-CoA levels should go back up again (as shown). The fluorescence (λₑₓ=405 nm, 485 nm; λₑₘ=514 nm) was measured by flow cytometry. The fluorescence was normalized by dividing F₄₈₈/F₄₀₅ and then (F₁−F₀)/F₀ where F₀ were cells that were not refed with glucose, n=3, *p<0.05.

FIG. 18 is a line graph showing the fluorescence of live E. coli that express either PancAce (sensor) or cpGFP that were either fed 28 mM glucose for 3 hours prior (“pre-fed”) or deprived of glucose for 3 hours prior (“pre-starved”). At time 0:00, the cells were given either phosphate buffered saline (“+PBS”) or 28 mM glucose (“+glucose”), and the fluorescence was measured by a plate reader (λₑₓ=405 nm, 485 nm; λₑₘ=514 nm) over 1 hour as shown on the X-axis. The fluorescence was normalized by dividing F₄₈₈/F₄₀₅ and then (F₁−F₀)/F₀ where F₀ is defined by the fluorescence from each population of cells prior to time 0:00, n=3. Only the cells that express the sensor and were pre-starved of glucose showed a large jump in signal, which is indicative of those cells having relatively low acetyl-CoA to start and then recovering their acetyl-CoA levels once they were given glucose.

FIG. 19 is a line graph showing the fluorescence of live E. coli that express PancAce (sensor) that were either fed 28 mM glucose for 3 hours prior (“pre-fed”) or deprived of glucose for 3 hours prior (“pre-starved”). At time 0:05, the cells were given 280 μM acetate, and the fluorescence was measured by a plate reader (λₑₓ=405 nm, 485 nm; λₑₘ=514 nm) over 1 hour as shown on the X-axis. The fluorescence was normalized by dividing F₄₈₈/F₄₀₅ and then (F₁-F₀)/F₀ where F₀ is defined by the fluorescence from each population of cells prior to time 0:05, n=3. Acetate can be an alternative source of acetyl-CoA for the cells, so here shows that if the cells have been deprived of glucose and have low acetyl-CoA levels, then they will use acetate to make acetyl-CoA. However, if the cells have had access to glucose (pre-fed), then the infusion of acetate causes no change in the cells because they continue to use the glucose that they have to produce acetyl-CoA.

FIG. 20 is a line graph showing the fluorescence of live E. coli that express PancAce (sensor) that were either fed 28 mM glucose or 280 μM acetate for 3 hours prior. At time 0:05, the cells were given either 28 mM glucose or 280 μM acetate as indicated in the figure legend, and the fluorescence was measured by a plate reader (λₑₓ=405 nm, 485 nm; λₑₘ=514 nm) over 1 hour as shown on the X-axis. The fluorescence was normalized by dividing F₄₈₈/F₄₀₅ and then (F₁−F₀)/F₀ where F₀ is defined by the fluorescence from each population of cells prior to time 0:05, n=3. This data shows that E. coli prefer glucose as their acetyl-CoA source compared to acetate.

FIG. 21 is a line graph showing the fluorescence of live E. coli that express PancAce (sensor) that were deprived of glucose for 3 prior with or without 28 mM 2-deoxyglucose (“−2-DG” or “+2-DG”). 2-DG was used to inhibit glycolysis, which is the metabolic process that converts glucose to acetyl-CoA. At time 0:05, the cells were given 28 mM glucose, and the fluorescence was measured by a plate reader (λₑₓ=405 nm, 485 nm; λₑₘ=514 nm) over 1 hour as shown on the X-axis. The fluorescence was normalized by dividing F₄₈₈/F₄₀₅ and then (F₁-F₀)/F₀ where F₀ is defined by the fluorescence from each population of cells prior to time 0:05, n=3. The data shows that in the presence of 2-DG, the cells are less able to recover acetyl-CoA levels because glycolysis is inhibited.

FIG. 22 is images showing fluorescence confocal microscopy of Hela cells that stably express PancAce (“sensor”) or cpGFP with a cytoplasmic localization sequence (“cyto”), nuclear localization sequence (“nuc”), or mitochondrial localization sequence (“mito”). The scale bars represent 80 μm. λₑₓ=485, λₑₘ=514 nm. This shows that the Hela cells are expressing the fluorescent protein constructs and that the protein translated from the constructs is going to the correct compartment corresponding to the localization sequence comprised by the protein.

FIG. 23A, FIG. 23B, and FIG. 23C are bar graphs showing the normalized sensor signal from live Hela cells that express the sensor in the nucleus (FIG. 23A), cytoplasm (FIG. 23B), or mitochondrion (FIG. 23C). In these experiments the Hela cells were deprived of different combinations of acetyl-CoA precursor nutrients and the effect on acetyl-CoA levels in the different compartments of the cell were measured. The cells were given either fetal bovine serum (FBS) and deprived of glucose (“+FBS/−glucose”), given dialyzed fetal bovine serum (“dFBS”) instead of FBS and given glucose (“+dFBS/+glucose), or given dFBS and deprived of glucose (“+dFBS/−glucose”) for 18 hours prior to imaging the cells by confocal fluorescence microscopy (λₑₓ=405 nm, 485 nm; λₑₘ=514 nm). To obtain the normalized signal, the signal from PancAce was normalized by dividing F₄₈₈/F₄₀₅, then F_{Pan}/F_{GFP}, then normalizing to the signal from cells that were given +FBS/+glucose for the same time interval (“F₀”): (F₁−F₀)/F₀. n=5-6. * p<0.05, ** p<0.005, *** p<0.0005, ****<0.0001. This data shows that the sensor can detect compartment-specific changes in acetyl-CoA in live cells.

FIG. 24 is bar graphs showing the normalized sensor signal from live Hela cells that express the sensor in the nucleus or cytoplasm. Similar to the E. coli experiments, here the cells were deprived of glucose and then refed with glucose to see whether the acetyl-CoA levels were recovered in the Hela cells after the Hela cells had access to glucose again. The cells were deprived of glucose for 18 hours and then given 4.5 g/L glucose for 1 hour or 3 hours prior to imaging the cells by confocal fluorescence microscopy (λₑₓ=405 nm, 485 nm; λₑₘ=514 nm). To obtain the normalized signal, the signal from PancAce was normalized by dividing F₄₈₈/F₄₀₅, then F_{Pan}/F_{GFP}, then normalizing to the signal from cells that were given +FBS/+glucose for the same time intervals (“F₀”): (F₁−F₀)/F₀. n=5-6. * p<0.05, ** p<0.005.

FIG. 25A, FIG. 25B, and FIG. 25C are bar graphs showing the normalized sensor signal from live Hela cells that express the sensor in the nucleus (FIG. 25A), cytoplasm (FIG. 25B), or mitochondrion (FIG. 25C). Different enzymes that produce or consume acetyl-CoA in different compartments in the cell are being knocked-down. The cells were transfected with siRNAs targeting the proteins indicated on the X-axis of the graph. The cells were imaged by confocal fluorescence microscopy (λₑₓ=405 nm, 485 nm; λₑₘ=514 nm) at 48 hours post-transfection. To obtain the normalized signal, the signal from PancAce was normalized by dividing F₄₈₈/F₄₀₅, then F_{Pan}/F_{GFP}, then normalizing to the signal from cells that were transfected with non-targeting siRNA for the same time interval (“F₀”): (F₁−F₀)/F₀. n=5-6. * p<0.05, ** p<0.005. These data show that compartmentalized acetyl-CoA level changes depend on the specific enzyme targeted.

FIG. 26 is images depicting a series of immunoblots that show the knockdown of the specified protein via the siRNAs that were used in the Hela cells in FIGS. 25A-25C. Site-specific antibodies were used for each siRNA target (“NT”=non-targeting siRNA). Actin is shown as a control for protein loading.

FIG. 27 is a bar graph showing the normalized sensor signal from live Hela cells that express the sensor in the nucleus or cytoplasm. The biochemical data herein (FIG. 11 and FIG. 12) indicated that the sensor might also bind to propionyl-CoA in addition to acetyl-CoA in a cell. It has been shown that branched-chain amino acids (“BCAAs”) deprivation causes propionyl-CoA levels to significantly increase in the nucleus of cells, but not acetyl-CoA levels. The cells were deprived of the BCAAs, isoleucine and valine, for 24 hours before the cells were imaged by confocal fluorescence microscopy (λₑₓ=405 nm, 485 nm; λₑₘ=514 nm). To obtain the normalized signal, the signal from PancAce was normalized by dividing F₄₈₈/F₄₀₅, then F_{Pan}/F_{GFP}, then normalizing to the signal from cells that were given BCAAs for the same time interval (“F₀”): (F₁−F₀)/F₀. n=5-6. * p<0.05, ** p<0.005. In the cytoplasm, both propionyl-CoA and acetyl-CoA levels decreased by about the same amount. These data indicate a reduction in the cytoplasm and not the nucleus, which implies that the sensor is in fact selective for acetyl-CoA in cells.

### DETAILED DESCRIPTION

Described herein are recombinant polypeptide biosensors that can be used to detect and/or quantify acetyl coenzyme A (acetyl-CoA). The recombinant polypeptide biosensors comprise an acetyl-CoA binding protein that is divided into first and second acetyl-CoA binding protein fragments and a fluorescent protein inserted between the first and second Acetyl-CoA binding protein fragments. Further described herein are methods of using the biosensor to detect and/or quantify acetyl-CoA and expression vectors comprising nucleic acids that encode the recombinant polypeptide biosensors.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Acetyl-CoA” or “acetyl COA” are abbreviations of acetyl coenzyme A. Acetyl-CoA has a number of physiological roles and is a component of cellular respiration that adds acetyl groups to biochemical reactions. These reactions are used in metabolizing proteins, carbohydrates, and lipids that provide energy sources in the forms of adenosine triphosphate (ATP), lactic acid, and ketone bodies. Acetyl-CoA also plays an important regulatory role in intracellular mechanisms and it is essential for energy production when fasting or starving.

“Acetyl-CoA biosensor,” “recombinant acetyl-CoA biosensor,” “biosensor,” or “sensor” are used interchangeably herein and refer to any of the recombinant acetyl-CoA biosensor polypeptides described herein. “ACoABP-X fusion protein” refers to a recombinant acetyl-CoA biosensor polypeptide where an acetyl-CoA binding protein (“ACoABP”) is divided into first and second acetyl-CoA binding protein fragments and a fluorescent protein (“X”) is inserted between the first and second acetyl-CoA binding protein fragments. For example, PanZ-cpGFP refers to a recombinant acetyl-CoA biosensor, as described herein, where the acetyl-CoA binding protein is “PanZ”, the PanZ is divided into first and second acetyl-CoA binding protein fragments and the circularly permuted green fluorescent protein (“cpGFP”) is inserted between the first and second acetyl-CoA binding protein fragments of the PanZ. “ACoABP (N #₁)-N₀₋₃-X-N₀₋₃-(N #2) AcCoABP,” is also used to refer to a recombinant acetyl-CoA biosensor described herein, where:
(a) “N” generally refers to amino acids;
(b) ACoABP (N #₁) refers to a first acetyl-CoA binding protein fragment comprising an N-terminal portion of an acetyl-CoA binding protein, where (N #₁) is the identity and position of the C-terminal amino acid of the first acetyl-CoA binding protein fragment at the position where the acetyl-CoA binding protein has been divided;
(c) (N #₂) AcCoABP refers to a second acetyl-CoA binding protein fragment comprising a C-terminal portion of the acetyl-CoA binding protein, where (N #₂) is the identity and position of the N-terminal amino acid of the second acetyl-CoA binding protein fragment at the position where the acetyl-CoA binding protein has been divided;
(d) X is a fluorescent protein inserted between the first and second acetyl-CaO binding protein fragments and attached to the C-terminus of the first acetyl-CoA binding protein fragment and the N-terminus of the second acetyl-CoA binding protein fragment; and
(e) “N₀₋₃” refers to an amino acid linker that is either a direct bond (i.e., N₀) or includes 1-3 amino acids (i.e., N₁₋₃).

For example, PanZ (R69)-GA-cpGFP-GA-(E70) PanZ refers to a recombinant acetyl-CoA polypeptide where the AcCoA binding protein is PanZ, the acetyl-CoA binding protein has been recombinantly divided between the Arg69 (R69) and glu70 (E70) of PanZ to form a first acetyl-CoA binding protein fragment (i.e., “PanZ (R69)”) and a second acetyl-CoA binding protein fragment (i.e., “(E70) PanZ”), a circularly permuted green fluorescent protein (i.e., “cpGFP”) has been recombinantly inserted between the first and second acetyl-CoA binding protein fragments. Specifically, in this example, the N-terminal end of the cpGFP is attached to R69 at the C-terminus of the first acetyl-CoA binding protein fragment by a two amino acid linker consisting of an N-terminal glycine and C-terminal alanine (“GA). Similarly, the C-terminal end of the cpGFP is attached to E70 at the N-terminus of the second acetyl-CoA binding protein fragment by a two amino acid linker consisting of an N-terminal glycine and a C-terminal alanine (“GA”).

The term “PancAce” is used herein to refer to the recombinant acetyl-CoA biosensor having the amino acid sequence of SEQ ID NO: 5.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Conservative amino acid substitution” as used herein refers to a substitution of an amino acid residue for another amino acid residue having similar biochemical properties. “Conservative” amino acid substitutions are those substitutions that do not substantially affect or decrease an activity of a polypeptide such as an acetyl-CoA binding domain or a fluorescent protein. A polypeptide can include one or more conservative substitutions up to and including 1-10 total conservative substitutions, 1% conservative substitutions, 5% conservative substitutions, 10% conservative substitutions, 15% conservative substitutions, 20% conservative substitutions, 25% conservative substitutions, 30% or more conservative substitutions, or any intervening value. Specific, non-limiting examples of a conservative substitution include the following shown in TABLE 1.

TABLE 1
Conservative Amino
Acid Substitutions

| Original | |
|---|---|
| Amino | Conservative |
| Acid | Substitutions |
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

While examples of polypeptide sequences are provided in the amino acid sequences attached to this application, not all variants of polypeptide sequences with all possible combinations of conservative amino acid substitutions encompassed by the disclosure are provided in the sequence listing. This table can be used in combination with the sequence listing to provide explicit examples of polypeptide sequences encompassed by the disclosure.

The terms “control,” “reference level,” and “reference” are used herein interchangeably. The reference level may be a predetermined value or range, which is employed as a benchmark against which to assess the measured result. “Control group” as used herein refers to a group of control subjects or cells. A control may be a subject or cell without a recombinant acetyl-coenzyme A (acetyl-CoA) biosensor polypeptide as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Fluorescent protein” as used herein refers to any protein characterized by a barrel structure that allows the protein to absorb light at one or more absorbance wavelength(s) and fluoresce (i.e., emit light) at one or more emission wavelength(s) in the visible spectrum. Fluorescent proteins may include, but are not limited to, green fluorescent proteins (GFPs), yellow fluorescent proteins (YFPs), blue fluorescent proteins (BFPs), red fluorescent proteins (RFPs) and cyan fluorescent proteins (CFPs), among others. The fluorescent proteins may be modified or derivatized to enhance fluorescence or GFPs. For example, enhanced fluorescent proteins may include amino acid mutations relative to corresponding wild type fluorescent proteins, that enhance the fluorescence of the protein. Numerous enhanced fluorescent proteins have been made and are well characterized in the art including, but not limited to enhanced GFPs (EGFPs), enhanced YFPs (EYFPs), enhanced BFPs (EBFPs), enhanced CFPs (ECFPs), and the like. The fluorescent protein also may be circularly permuted by fusing the original N- and C-termini of a fluorescent protein together, either directly or using a peptide linker, and forming new termini near the chromophore while still retaining a similar 3-dimensional structure as the original fluorescent protein. Circularly permuted fluorescent proteins also are well known in the art and include, but are not limited to, circularly permuted GFPs (cpGFPs), circularly permuted YFPs (cpYFPs), circularly permuted BFPs (cpBFPs), circularly permuted CFPs (cpCFPs) and circularly permuted RFPs (cpRFPs), among others.

“Fusion protein” as used herein refers to a chimeric protein created through the joining of two or more genes that originally coded for separate proteins. The translation of the fusion gene results in a single polypeptide with functional properties derived from each of the original proteins. A fusion protein may also be a recombinant protein.

“Genetic construct” as used herein refers to DNA or RNA molecules that comprise a polynucleotide that encodes a protein. The coding sequence includes initiation and termination signals operably linked to regulatory elements including a promoter and polyadenylation signal capable of directing expression in cells or cells of a subject to whom the nucleic acid molecule is administered. As used herein, the term “expressible form” refers to gene constructs that contain the necessary regulatory elements operable linked to a coding sequence that encodes a protein such that when present in a cell or subject, the coding sequence will be expressed.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Label” or “tag” as used interchangeably herein refer to any substance capable of aiding a machine, detector, sensor, device, column, or enhanced or unenhanced human eye from differentiating a labeled nucleotide, polynucleotide, polypeptide, or composition from an unlabeled nucleotide, polynucleotide, polypeptide, or composition. Labels may be used for any of a number of purposes and one skilled in the art will understand how to match the proper label with the proper purpose. Examples of uses of labels include purification of biomolecules, identification of biomolecules, detection of the presence of biomolecules, detection of protein folding, and localization of biomolecules within a cell, tissue, or organism. Examples of labels include but are not limited to: radioactive isotopes (such as carbon-14 or ¹⁴C) or chelates thereof; dyes (fluorescent or nonfluorescent), stains, enzymes, nonradioactive metals, magnets, protein tags, any antibody epitope, any specific example of any of these; any combination between any of these, or any label now known or yet to be disclosed. A label may be covalently attached to a biomolecule or bound through hydrogen bonding, Van Der Waals, or other forces. A label may be covalently or otherwise bound to the N-terminus, the C-terminus, or any amino acid of a polypeptide, or the 5′ end, the 3′ end or any nucleic acid residue in the case of a polynucleotide.

An example of a label is a protein tag. A protein tag comprises a sequence of one or more amino acids that may be used as a label as discussed above, particularly for use in protein purification. In some examples, the protein tag is covalently bound to the polypeptide. It may be covalently bound to the N-terminal amino acid of a polypeptide, the C-terminal amino acid of a polypeptide, or any other amino acid of the polypeptide. The protein tag may be encoded by a polynucleotide sequence that is immediately 5′ of a nucleic acid sequence coding for the polypeptide such that the protein tag is in the same reading frame as the nucleic acid sequence encoding the polypeptide. Protein tags may be used for all of the same purposes as labels listed above and are well known in the art. Examples of protein tags include chitin binding protein (CBP), maltose binding protein (MBP), glutathione-S-transferase (GST), poly-histidine (His), thioredoxin (TRX), FLAG® tag, TEV cleavage site, V5, c-Myc, human influenza hemagglutinin (HA) tag, a nuclear export signal (NES), a nuclear localization signal or nuclear localization sequence (NLS), a cytoplasmic localization signal or cytoplasmic localization sequence (CLS), and a mitochondrial localization signal or mitochondrial localization sequence (MLS), and the like.

A His-tag facilitates purification and binding to on metal matrices, including nickel matrices, including nickel matrices bound to solid substrates such as agarose plates or beads, glass plates or beads, or polystyrene or other plastic plates or beads. Other protein tags include BCCP, calmodulin, Nus, Thioredoxin, Streptavidin, SBP, and Ty, or any other combination of one or more amino acids that can work as a label described above.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, fluorescent proteins, and receptors. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. A domain of a polypeptide or protein may be any part of a protein that exhibits a particular defined structure and/or mediates a particular protein function. An example of a domain is the acetyltransferase (GNAT) domain of PanZ (PanD regulatory factor). Exemplary domains include domains with acetyl-CoA binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. A motif may include 3, 4, 5, 6, or 7 sequential amino acids. A domain may be comprised of a series of the same type of motif.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, human U6 (hU6) promoter, and CMV IE promoter.

The term “recombinant” when used with reference to, for example, a cell, nucleic acid, protein, or vector, indicates that the cell, nucleic acid, protein, or vector has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Sample” or “test sample” as used herein can mean any sample in which the presence and/or level of a target is to be detected or determined or any sample comprising a recombinant acetyl-CoA biosensor polypeptide or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” as used herein refers to any vertebrate or invertebrate, including, but not limited to, a mammal that wants or is in need of the herein described compositions or methods and bacteria cells. The subject may be a human or a non-human. The subject may be a cell. The subject may be a bacterial cell such as, but not limited to, Escherichia coli (E. Coli). The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to bind acetyl-CoA and emit a fluorescent signal upon binding acetyl-CoA. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome, or yeast artificial chromosome. A vector may be a DNA or RNA vector. A vector may be a self-replicating extrachromosomal vector, and preferably, is a DNA plasmid. For example, the vector may encode a recombinant acetyl-CoA biosensor polypeptide.

### 2. Recombinant Acetyl-Coenzyme A Biosensor Polypeptides

Provided herein are recombinant acetyl-coenzyme A (acetyl-CoA) biosensor polypeptides that can detect free acetyl-CoA in solution, as well as in a cell. The recombinant acetyl-CoA biosensor polypeptide may include an acetyl-CoA binding protein and a fluorescent protein. The recombinant acetyl-CoA biosensor polypeptide may selectively bind acetyl-CoA. This binding causes a specific conformational change in the biosensor and results in a change in fluorescence emission. The binding of acetyl-CoA may induce a change in the fluorescence of the fluorescent protein. This change in fluorescence allows for detection of acetyl-CoA. The acetyl-CoA binding protein may have an amino acid sequence at least 40%, at least 45%, at least 50%, at least 55%, at least 60%, at least 65%, at least 70%, at least 75%, at least 80%, at least 85%, at least 90%, at least 95%, or at least 99% identical to SEQ ID NO: 1. In some embodiments, the acetyl-CoA binding protein may have an amino acid sequence of SEQ ID NO: 1. The acetyl-CoA binding protein may be derived from Escherichia coli (E. Coli), such as the acetyl-CoA binding protein PanZ, which may also be referred to as “PanM” in the art. The acetyl-CoA binding protein may also be derived from enterobacterial species including Shigella, Salmonella, Klebsiella, and Yersinia. As discussed above, the acetyl-CoA binding protein may be divided into a first acetyl-CoA binding protein fragment including an N-terminal portion of the acetyl-CoA binding protein, and a second acetyl-CoA binding protein fragment including a C-terminal portion of the acetyl-CoA binding protein. The first acetyl-CoA binding protein fragment and the second acetyl-CoA binding protein fragment collectively may include all of the amino acids of the acetyl-CoA binding protein. The first acetyl-CoA binding protein fragment comprises the amino acids at or near the N-terminal portion of the acetyl-CoA binding protein and is attached to the N-terminal end of the fluorescent protein, while the second acetyl-CoA binding fragment comprises the amino acids at or near the C-terminal portion of the acetyl-CoA binding protein and is attached to the C-terminal end of the fluorescent protein.

The fluorescent protein may include any protein characterized by a barrel structure that allows the protein to absorb light at one or more absorbance wavelength(s) and fluoresce (i.e., emit light) at one or more emission wavelength(s) in the visible spectrum. As discussed in more detail above, fluorescent proteins may include, but are not limited to, GFPs, YFPs, BFPs, RFPs, CFPs, EGFPs, EYFPs, EBFPs, ECFPs, cpGFPs, cpYFPs, cpBFPs, cpCFPs, and cpRFPs, among others. In some embodiments, the fluorescent protein may be a cpGFP, a cpYFP, or a cpBFP. In some embodiments, the cpGFP may have an amino acid sequence of SEQ ID NO: 2, the cpYFP may have an amino acid sequence of SEQ ID NO: 3, and the cpBFP may have an amino acid sequence of SEQ ID NO: 4.

The fluorescent protein is inserted between the first acetyl-CoA binding protein fragment and the second acetyl-CoA binding protein fragment. Specifically, the fluorescent protein is attached to a C-terminus of the first acetyl-CoA binding protein fragment and an N-terminus of the second acetyl-CoA binding protein fragment. In some embodiments, the C-terminus of the first acetyl-CoA binding protein fragment may be an arginine at position 69 (Arg69) of the acetyl-CoA binding protein and the N-terminus of the second acetyl-CoA binding protein fragment may be a glutamic acid at position 70 (Glu70) of the acetyl-CoA binding protein. In some embodiments, the C-terminus of the first acetyl-CoA binding protein fragment may be a tryptophan at position 23 (Trp23) of the acetyl-CoA binding protein and the N-terminus of the second acetyl-CoA binding protein fragment may be a proline at position 24 (Pro24) of the acetyl-CoA binding protein. In some embodiments, the C-terminus of the first acetyl-CoA binding protein fragment may be a valine at position 71 (Val71) of the acetyl-CoA binding protein and the N-terminus of the second acetyl-CoA binding protein fragment may be a threonine at position 72 (Thr72) of the acetyl-CoA binding protein. In some embodiments, the C-terminus of the first acetyl-CoA binding protein fragment may be an aspartic acid at position 99 (Asp99) of the acetyl-CoA binding protein and the N-terminus of the second acetyl-CoA binding protein fragment may be an alanine at position 100 (Ala100) of the acetyl-CoA binding protein. In some embodiments, the C-terminus of the first acetyl-CoA binding protein fragment may be an aspartic acid at 104 (Asp104) of the acetyl-CoA binding protein and the N-terminus of the second acetyl-CoA binding protein fragment may be an arginine at position 105 (Arg105) of the acetyl-CoA binding protein. In some embodiments, the C-terminus of the first acetyl-CoA binding protein fragment may be a glycine at position 116 (Gly116) of the acetyl-CoA binding protein and the N-terminus of the second acetyl-CoA binding protein fragment may be a phenylalanine at position 117 (Phe117) of the acetyl-CoA binding protein.

In some embodiments, the N-terminus of the fluorescent protein may be directly attached to the C-terminus of the first acetyl-CoA binding protein fragment (i.e., by a peptide bond). In other embodiments, the N-terminus of the fluorescent protein may be attached to the C-terminus the first acetyl-CoA binding protein fragment by a first amino acid linker (which also may be referred to as a peptide linker) that is from 1-3 amino acids in length. In some embodiments, the first amino acid linker may be selected from the group consisting of a Gly linker, a Gly-Ala linker, an Ala-Ser linker, and a Gly-Ala-Ser linker.

In some embodiments, the C-terminus of the fluorescent protein may be directly attached to the N-terminus of the second acetyl-CoA binding protein fragment (i.e., by a peptide bond). In other embodiments, the C-terminus of the fluorescent protein may be attached to the N-terminus the second acetyl-CoA binding protein fragment by a second peptide linker that is from 1-3 amino acids in length. In some embodiments, the second amino acid linker may be selected from the group consisting of a Gly linker, a Gly-Ala linker, an Ala-Ser linker, and a Gly-Ala-Ser linker.

In some embodiments, the first amino acid linker may be Gly-Ala and the second amino acid linker may be Gly-Ala. In some embodiments, the first amino acid linker may be Ala-Ser and the second amino acid linker may be Ala-Ser. In some embodiments, the first amino acid linker may be Gly-Ala-Ser and the second amino acid linker may be Gly. In some embodiments, the C-terminus of the first acetyl-CoA binding protein fragment may be directly attached to the N-terminus of the fluorescent protein and the N-terminus of the second acetyl-CoA binding protein fragment may be directly attached to the C-terminus of the fluorescent protein. In some embodiments, the C-terminus of the first acetyl-CoA binding protein fragment may be directly attached to the N-terminus of the fluorescent protein and the second amino acid linker may be Gly-Ala-Ser. In some embodiments, the first amino acid linker may be Gly-Ala-Ser and the N-terminus of the second acetyl-CoA binding protein fragment may be directly attached to the fluorescent protein. In some embodiments, the first linker may be Gly-Ala and the N-terminus of the second acetyl-CoA binding protein fragment may be directly attached to C-terminus of the fluorescent protein. In some embodiments, the first linker may be Gly and the second linker may be Gly-Ala-Ser.

In some embodiments, the recombinant acetyl-CoA biosensor polypeptide may have the amino acid sequence of SEQ ID NO: 5. In some embodiments, the recombinant acetyl-CoA biosensor polypeptide may have the amino acid sequence of SEQ ID NO: 6. In some embodiments, the recombinant acetyl-CoA biosensor polypeptide may have the amino acid sequence of SEQ ID NO: 7. In some embodiments, the recombinant acetyl-CoA biosensor polypeptide may have the amino acid sequence of SEQ ID NO: 8. In some embodiments, the recombinant acetyl-CoA biosensor polypeptide may have the amino acid sequence of SEQ ID NO: 9. In some embodiments, the recombinant acetyl-CoA biosensor polypeptide may have the amino acid sequence of SEQ ID NO: 10. In some embodiments, the recombinant acetyl-CoA biosensor polypeptide may have the amino acid sequence of SEQ ID NO: 11. In some embodiments, the recombinant acetyl-CoA biosensor polypeptide may have the amino acid sequence of SEQ ID NO: 12. In some embodiments, the recombinant acetyl-CoA biosensor polypeptide may have the amino acid sequence of SEQ ID NO: 13. In some embodiments, the recombinant acetyl-CoA biosensor polypeptide may have the amino acid sequence of SEQ ID NO: 14. In some embodiments, the recombinant acetyl-CoA biosensor polypeptide may have the amino acid sequence of SEQ ID NO: 15. In some embodiments, the recombinant acetyl-CoA biosensor polypeptide may have the amino acid sequence of SEQ ID NO: 16. In some embodiments, the recombinant acetyl-CoA biosensor polypeptide may have the amino acid sequence of SEQ ID NO: 17. In some embodiments, the recombinant acetyl-CoA biosensor polypeptide may have the amino acid sequence of SEQ ID NO: 18. In some embodiments, the recombinant acetyl-CoA biosensor polypeptide may have the amino acid sequence of SEQ ID NO: 19. A particular embodiment of the present disclosure provides a recombinant acetyl-CoA biosensor polypeptide having the amino acid sequence of SEQ ID NO: 5 and having selectivity for acetyl-CoA.

A recombinant acetyl-CoA biosensor polypeptide described herein may include one or more additional elements such as one or more of tags (e.g. a histidine tag, a tobacco etch virus protease (TEV) cleavage site, a FLAG® tag, a human influenza hemagglutinin (HA) tag), localization sequences (e.g., a nuclear export signal (NES), a nuclear localization signal (NLS), a cytoplasmic localization signal (CLS), and a mitochondrial localization signal (MLS)), labels (e.g., a fluorescent label), modified amino acids, artificial amino acids, and the like. The additional element(s) may be at the N-terminal portion of the acetyl-CoA binding protein and/or the C-terminal portion of the acetyl-CoA binding protein.

The syntheses of the recombinant acetyl-CoA biosensor polypeptides described herein can be carried out by any method known in the art. For example, the recombinant acetyl-CoA biosensor polypeptides described herein may be produced by recombinant methods where the recombinant acetyl-CoA biosensor polypeptide may be produced through recombinant DNA technology. This may involve inserting DNA encoding the recombinant acetyl-CoA biosensor polypeptide into bacterial or mammalian cells, expressing the recombinant acetyl-CoA biosensor polypeptide in the cells, and then purifying the recombinant acetyl-CoA biosensor polypeptide from the cells using methods known in the art.

### 3. GENETIC CONSTRUCTS

The recombinant acetyl-CoA biosensor polypeptide may be encoded by or comprised within a genetic construct. The genetic construct, such as a plasmid or expression vector, may comprise a nucleic acid that encodes the recombinant acetyl-CoA biosensor polypeptide. In some embodiments, an expression vector may comprise a nucleic acid that encodes a recombinant acetyl-CoA biosensor polypeptide described herein and a promoter operably linked to the nucleic acid.

Genetic constructs may include polynucleotides such as vectors and plasmids. The vector may be an expression vector or system to produce protein by routine techniques and readily available starting materials including Sambrook et al., Molecular Cloning and Laboratory Manual, Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the recombinant acetyl-CoA biosensor polypeptide and may further comprise an initiation codon, which may be upstream of the recombinant acetyl-CoA biosensor polypeptide coding sequence, and a stop codon, which may be downstream of the recombinant acetyl-CoA biosensor polypeptide coding sequence. The genetic construct may include more than one stop codon, which may be downstream of the recombinant acetyl-CoA biosensor polypeptide coding sequence. A stop codon may be in-frame with a coding sequence in the recombinant acetyl-CoA biosensor polypeptide. The genetic construct may include one or more stop codons that are out of frame of a coding sequence in the recombinant acetyl-CoA biosensor polypeptide. The initiation and termination codon may be in frame with the recombinant acetyl-CoA biosensor polypeptide coding sequence.

The vector may also comprise a promoter that is operably linked to the recombinant acetyl-CoA biosensor polypeptide coding sequence. The promoter may be a constitutive promoter, an inducible promoter, a repressible promoter, or a regulatable promoter. The promoter may be a ubiquitous promoter. The promoter may be a tissue-specific or organelle-specific promoter. The promoter operably linked to the recombinant acetyl-CoA biosensor polypeptide coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metallothionein.

The genetic construct may also comprise a polyadenylation signal, which may be downstream of the recombinant acetyl-CoA biosensor polypeptide coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal.

Coding sequences in the genetic construct may be optimized for stability and high levels of expression.

The genetic construct may also comprise an enhancer upstream of the recombinant acetyl-CoA biosensor polypeptide coding sequence. The enhancer may be necessary for DNA expression. The enhancer may be a viral enhancer such as one selected from CMV, HA, RSV, or EBV. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as green fluorescent protein (“GFP”) and/or a selectable marker, such as hygromycin (“Hygro”).

The genetic construct may be useful for transfecting, transducing, or transforming cells with a nucleic acid encoding the recombinant acetyl-CoA biosensor polypeptide, wherein the transfected, transduced, or transformed host cell may be cultured and maintained under conditions wherein expression of the recombinant acetyl-CoA biosensor polypeptide takes place. The genetic construct may be transformed, transfected, or transduced into a cell. The genetic construct may be formulated into any suitable type of delivery vehicle including, for example, a viral vector, lentiviral vector, adeno-associated virus (AAV) vector, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed, transfected, or transduced with a recombinant acetyl-CoA biosensor polypeptide or component thereof as detailed herein. Suitable cell types are detailed herein. In some embodiments, the cell is a bacterial cell.

a. Viral Vectors

A genetic construct may be a viral vector. Further provided herein is a viral delivery system. Viral delivery systems may include, for example, lentivirus, retrovirus, adenovirus, cytomegalovirus (CMV), mRNA electroporation, or nanoparticles. In some embodiments, the vector is a lentiviral vector. In some embodiments, the viral vector is an adeno-associated virus (AAV) vector. In some embodiments, the viral vector is a CMV vector.

Lentiviral vectors may be used to deliver the recombinant acetyl-CoA biosensor polypeptide using various construct configurations. AAV vectors may be used to deliver the recombinant acetyl-CoA biosensor polypeptide using various construct configurations. CMV vectors may be used to deliver the recombinant acetyl-CoA biosensor polypeptide using various construct configurations. In some embodiments, the lentiviral vector is a modified lentiviral vector. In some embodiments, the AAV vector is a modified AAV vector. In some embodiments, the CMV vector is a modified CMV vector. The AAV vector or modified AAV vector may be based on one or more of several capsid types, including AAV1, AAV2, AAV5, AAV6, AAV8, and AAV9.

### 4. COMPOSITIONS

Further provided herein are compositions comprising the above-described recombinant acetyl-CoA biosensor polypeptides. In some embodiments, the composition may comprise from about 0.1 μM to about 10 μM, about 0.5 μM to about 10 μM, about 1 μM to about 10 μM, about 1.5 μM to about 10 μM, about 2 μM to about 10 μM, about 2.5 μM to about 10 μM, about 3 μM to about 10 μM, about 3.5 μM to about 10 μM, about 4 μM to about 10 μM, about 4.5 μM to about 10 μM, about 5 μM to about 10 μM, about 5.5 μM to about 10 μM, about 6 μM to about 10 μM, about 6.5 μM to about 10 μM, about 7 μM to about 10 μM, about 7.5 μM to about 10 μM, about 8 μM to about 10 μM, about 8.5 μM to about 10 μM, about 9 μM to about 10 μM, about 9.5 μM to about 10 μM, about 0.1 μM to about 9.5 μM, about 0.1 μM to about 9 μM, about 0.1 μM to about 8.5 μM, about 0.1 μM to about 8 μM, about 0.1 μM to about 7.5 μM, about 0.1 μM to about 7 μM, about 0.1 μM to about 6.5 μM, about 0.1 μM to about 6 μM, about 0.1 μM to about 5.5 μM, about 0.1 μM to about 5 μM, about 0.1 μM to about 4.5 μM, about 0.1 μM to about 4 μM, about 0.1 μM to about 3.5 μM, about 0.1 μM to about 3 μM, about 0.1 μM to about 2.5 μM, about 0.1 μM to about 2 μM, about 0.1 μM to about 1.5 μM, or about 0.1 μM to about 1 μM of the recombinant acetyl-CoA biosensor polypeptide or recombinant DNA encoding the recombinant acetyl-CoA biosensor. In some embodiments, the composition may comprise about 1 μM of the recombinant acetyl-CoA biosensor polypeptide or recombinant DNA encoding the recombinant acetyl-CoA biosensor. The recombinant acetyl-CoA biosensor polypeptides or recombinant DNA encoding the recombinant acetyl-CoA biosensor as detailed herein may be formulated into compositions in accordance with standard techniques well known to those skilled in the art. The compositions can be formulated according to the mode of administration to be used. The compositions may be sterile, pyrogen free, and particulate free. An isotonic formulation may also be used. Generally, additives for isotonicity may include sodium chloride, dextrose, mannitol, sorbitol, and lactose. In some cases, isotonic solutions such as phosphate buffered saline may be preferred. Stabilizers may include gelatin and albumin.

### 5. ADMINISTRATION

The recombinant acetyl-CoA biosensor polypeptides disclosed herein or compositions comprising the same may be administered or provided to a cell. The cell may be a bacterial cell. The cell may be in a subject. The recombinant acetyl-CoA biosensor polypeptides disclosed herein or compositions comprising the same may be administered or delivered to an organelle of a cell, such as the nucleus, mitochondria, cytoplasm, and the like. Methods of introducing a peptide into a host cell are known in the art, and any known method can be used to introduce a recombinant acetyl-CoA biosensor polypeptide into a cell. Suitable methods may include, for example, transformation, transduction, transfection, electroporation, direct microinjection, and the like.

The recombinant acetyl-CoA biosensor polypeptides as detailed herein or the compositions comprising the same may be administered to a subject. Such compositions can be administered in dosages and by techniques well known to those skilled in the medical arts taking into consideration such factors as the age, sex, weight, and condition of the particular subject, and the route of administration. The presently disclosed recombinant acetyl-CoA biosensor polypeptides or compositions comprising the same may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, intraarticular, or combinations thereof. The recombinant acetyl-CoA biosensor polypeptides or compositions comprising the same may be delivered to a subject by several technologies including liposome mediated, nanoparticle facilitated, recombinant vectors such as recombinant lentivirus, recombinant adenovirus, and recombinant adenovirus associated virus. For veterinary use, the recombinant acetyl-CoA biosensor polypeptides or compositions comprising the same may be administered as a suitably acceptable formulation in accordance with normal veterinary practice. The veterinarian may readily determine the dosing regimen and route of administration that is most appropriate for a particular animal. The recombinant acetyl-CoA biosensor polypeptides or compositions comprising the same may be administered by traditional syringes, or other physical methods such as electroporation (“EP”), “hydrodynamic method”, or ultrasound.

Upon delivery of the presently disclosed recombinant acetyl-CoA biosensor polypeptides as detailed herein, or the compositions comprising the same, the transfected, transduced, transformed cells may express the recombinant acetyl-CoA biosensor polypeptide.

a. Cell Types

Any of the delivery methods and/or routes of administration detailed herein can be utilized with a myriad of cell types. Further provided herein is a cell transformed, transfected, or transduced with a recombinant acetyl-CoA biosensor polypeptide as detailed herein. For example, provided herein is a cell comprising an isolated polynucleotide encoding a recombinant acetyl-CoA biosensor polypeptide as detailed herein. In some embodiments, the cell may be a bacterial cell such as, but not limited to, E. Coli, Shigella, Salmonella, Klebsiella, and Yersinia. In some embodiments, the cell may be a yeast such as Saccharomyces cerevisiae. In some embodiments, the cell may be an immune cell. Immune cells may include, for example, lymphocytes such as T cells and B cells and natural killer (NK) cells, innate immune cells, adaptive immune cells, NKT cells, IFN-γ producing killer dendritic cells (IKDC), memory T cells (TCMs), and effector T cells (TEs). The cell may be a stem cell such as a human stem cell, an embryonic stem cell, a hematopoietic stem cell, an induced pluripotent stem cell (iPSC), stem cell-derived cell types such as neurons. The cell may be a primary cell such as a neuron, a muscle cell, a kidney cell, and the like. Cells may further include, but are not limited to, immortalized myoblast cells, dermal fibroblasts, bone marrow-derived progenitors, skeletal muscle progenitors, human skeletal myoblasts, CD133+ cells, mesoangioblasts, cardiomyocytes, hepatocytes, chondrocytes, mesenchymal progenitor cells, hematopoietic stem cells, smooth muscle cells, and MyoD-or Pax7-transduced cells. The cell may be a cancer cell. The cell may be a cell from a cell line such as a Human Embryonic Kidney (HEK) 293 cell.

### 6. METHODS

a. Methods of Detecting Acetyl-CoA

Provided herein are methods of detecting and/or quantifying acetyl-CoA in a sample. The methods may include contacting a sample with the recombinant acetyl-CoA biosensor polypeptide described herein and exciting the recombinant acetyl-CoA biosensor polypeptide in the sample at an excitation wavelength. The excitation wavelength may be from about 460 nm to about 490 nm. In a particular embodiment, the excitation wavelength may be 485 nm. After excitation of the recombinant acetyl-CoA biosensor polypeptide, the method may include measuring a fluorescence intensity of the recombinant acetyl-CoA biosensor polypeptide in the sample at an emission wavelength. The emission wavelength may be from about 513 nm to about 540 nm. In a particular embodiment, the emission wavelength may be 514 nm. The method may further includes comparing the fluorescence intensity to a standard curve, wherein the fluorescence intensity correlates with a concentration of acetyl-CoA in the sample. The standard curve may be generated by exciting the recombinant acetyl-CoA biosensor polypeptide in one or more control samples at the excitation wavelength and measuring a fluorescence intensity of the recombinant acetyl-CoA biosensor polypeptide in the one or more control samples at the emission wavelength. The standard curve may be based upon (i) one or more control samples comprising the recombinant acetyl-CoA biosensor polypeptide as described herein and without acetyl-CoA and (ii) one or more samples comprising the recombinant acetyl-CoA biosensor polypeptide as described herein and a known concentration (or series of known concentrations) of acetyl-CoA. The pH of the sample may be maintained at a pH of 6.5-8.0.

b. Methods of Monitoring Acetyl-CoA Activity in a Cell

Provided herein are methods of monitoring acetyl-CoA activity in a cell. The methods may include providing a cell with a recombinant acetyl-CoA biosensor polypeptide described herein and exciting the recombinant acetyl-CoA biosensor polypeptide in the cell at a first excitation wavelength between about 400 nm and about 430 nm while measuring a first fluorescence intensity at an emission wavelength between about 513 nm and about 540 nm. In a particular embodiment, the first excitation wavelength may be 405 nm. In another particular embodiment, the emission wavelength may be 514 nm. The method may further include exciting the recombinant acetyl-CoA biosensor polypeptide in the cell at a second excitation wavelength between about 460 nm and about 490 nm while measuring a second fluorescence intensity at the emission wavelength. In a particular embodiment, the second excitation wavelength may be 485 nm. The second fluorescence intensity may be normalized based on the first fluorescence intensity. Normalizing may include dividing the second fluorescence intensity by the first fluorescence intensity.

The cell may be treated with an acetyl-CoA precursor or nutrient affecting the function of the cell. Then, the normalized fluorescence intensity of the cell may be compared to the normalized fluorescence intensity of a control cell that did not receive the acetyl-CoA precursor or the nutrient. The acetyl-CoA precursor or nutrient may be one or more of glucose, fatty acids (e.g., fatty acyl CoA, octanoate, and palmitate), amino acids (e.g., glutamine, isoleucine, and valine), acyl CoA dehydrogenase, mono- and dicarboxylates (e.g., acetate, lactate, and alpha-ketoglutarate), and ketone bodies (e.g., acetoacetate and 3-beta-hydroxybutyrate).

The method may further comprise determining where the acetyl-CoA is localized in the cell by using a NES, a NLS, a CLS, or a MLS attached to the recombinant acetyl-CoA biosensor polypeptide as described herein.

### 7. KITS

Provided herein is a kit, which may be used to detect, quantify, monitor activity of, determine the presence of, and/or determine the location of acetyl-CoA. The kit comprises genetic constructs or a composition comprising the same, as described above, and instructions for using said composition. In some embodiments, the kit may comprise at least one genetic construct comprising a polynucleotide sequence that encodes a recombinant acetyl-CoA biosensor polypeptide described herein, wherein the polynucleotide may, for example, comprise a nucleic acid sequence selected from SEQ ID NOS: 24-38, SEQ ID NOS: 62-76, SEQ ID NO: 83, SEQ ID NO: 85, and SEQ ID NO: 87, a complement thereof, a variant thereof, or a fragment thereof. The kit may comprise at least one recombinant acetyl-CoA biosensor polypeptide comprising, for example, an amino acid sequence selected from SEQ ID NOS: 5-19, SEQ ID NOS: 43-57, SEQ ID NO: 77, SEQ ID NO: 79, and SEQ ID NO: 81, a complement thereof, a variant thereof, or fragment thereof. The kit may further include instructions for using the genetic construct or the recombinant acetyl-CoA biosensor polypeptide.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### 8. EXAMPLES

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Materials and Methods

Molecular Cloning. All PCR was done with Q5® High-Fidelity DNA Polymerase kit (New England Biolabs (NEB), Ipswich, MA) according to manufacturer's protocols. All primers were ordered from University of Utah Core Labs (Salt Lake City, UT). All plasmids were sequence verified by GENEWIZ®.

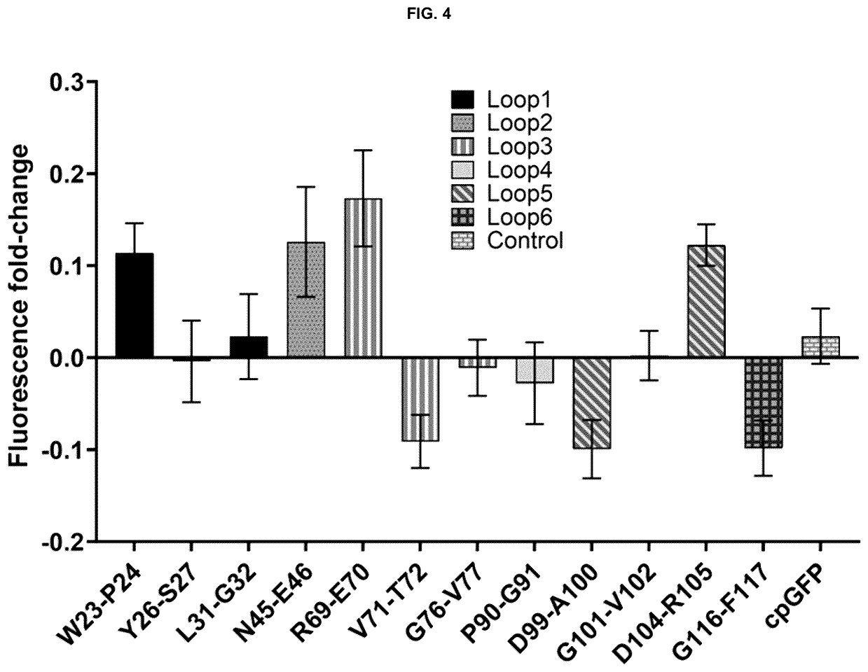

cpGFP Insertion Sites Screen. The E. coli PanZ gene was purchased from Integrated DNA Technologies (IDT, Coralville, IA) and was subcloned into the pET30A vector using the N-terminal 6× histidine tag followed by a TEV cleavage site (HTSD1.10). The cpGFP gene was Addgene 186790 and subcloned into the pET30A vector using the N-terminal 6× histidine tag followed by a TEV cleavage site (HTSD1.00). cpGFP was inserted into the PanZ gene between the amino acids as shown in FIG. 4, based off of recombinant protein sequence (based off of PanZ sequence), with an AS amino acid linker on the N and C-terminal ends of cpGFP using NEBuilder® HiFi DNA Assembly Master Mix (NEB) (HTSD6.00-6.13).

Linker Length Screen. cpGFP (HTSD1.00) was inserted into 6× histidine tagged PanZ (HTSD1.10) after amino acid R69 with all 16 possible combinations of N- and C-terminal linkers with amino acid sequences GAS, GA, G, no linker using NEBuilder® HiFi DNA Assembly Master Mix (NEB) (HTSD8.00-8.15).

cpGFP Mutation to Other Color Fluorophores. Quick Change Mutagenesis was done to cpGFP (HTSD1.00) to generate point mutants cpYFP (HTSD1.01) and cpBFP (HTSD1.02). NEBuilder® HiFi DNA Assembly Master Mix (NEB) was then used to insert them into 6x histidine tagged PanZ (HTSD1.10) with GA amino acid linkers on the N- and C-terminal of the R69-E70 position. This yielded Banana PANcACe (HTSD8.05Y) and Blueberry PANcACe (HTSD8.05B).

Lentiviral vectors. PancAce and cpGFP were subcloned from the pet30A plasmid into the pLJM1 plasmid (Addgene 19319) using the NEBuilder® Hifi DNA Assembly. The localization tags were subcloned from plasmids (Addgene 186787, 186788, and 186789).

Recombinant Protein Preparation. PanZ, cpGFP, cpYFP, cpBFP, and all sensor construct plasmids were transformed into BL21 Rosetta (DE3) cells for expression. Glycerol stocks of each construct were saved at −80° C. Glycerol stocks were used to inoculate 10 mL LB media with kanamycin. Cultures were grown at 37° C. overnight then used to inoculate 1 L LB with kanamycin. The 1 L cultures were grown at 37° C. to OD600=0.6. The temperature was then turned to 18° C. and flasks were induced with 0.5 mM IPTG for 18 hrs. Bacteria were pelleted at 4000 g for 30 minutes. The pellet was then purified or stored at −80° C. until purification. The pellet from 1 L of culture was resuspended in lysis buffer (50 mM Tris pH 7.5, 500 mM NaCl, 10 mM imidazole) then sonicated for 1 minute on, 3 minutes off with duty cycle 50%. Clarified lysates were obtained by spinning lysates at 17000 g for 30 minutes. Clarified lysates were then run over 1 mL of equilibrated Ni-resin and washed with 30 mL wash buffer (50 mM Tris pH 7.5, 50 mM NaCl, 50 mM imidazole). Recombinant proteins were then eluted with 5, 1 mL aliquots of elution buffer (50 mM Tris pH 7.5, 500 mM NaCl, 500 mM imidazole). Aliquots were then pooled, and buffer exchanged in (EMD Millipore, Burlington, MA; 30 kDa MWCO) into storage buffer (50 mM Tris pH 7.5, 150 mM NaCl, 10% glycerol). Proteins were concentrated to ˜100 μM then aliquoted and stored at −80° C. until use.

Surface Plasmon Resonance. Acetyl-CoA and CoA binding to PanZ-CFP were analyzed via SPR using a MASS-1 instrument (Bruker Daltonics, Billerica, MA). Independent experiments were performed using freshly prepared PanZ-CFP, Acetyl-CoA, and CoA stocks on two separate days (surfaces 1 & 2). HLC200M sensor surfaces (Xantec Bioanalytics, Düsseldorf, Germany) were activated with EDC/NHS, and then 250 nM PanZ-CFP ligand was captured on the sensor “Spot B” at 10 μL/minute at 2020 RU (surface 1) and 2360 RU (surface 2). The surface was then blocked with 1 μM ethanolamine. The experimental running buffer was 50 mM Tris pH 7.5, 150 mM NaCl, 0.05% Tween-20 for surface 1 replicates and 200 mM Tris pH 7.5, 150 mM NaCl, 0.05% Tween-20 for surface 2 replicates. Experiments were performed at room temperature with a flowrate of 30 μL/minute. A three-fold dilution series (81-0.33 μM) with 1 minute association and 2 minutes dissociation was run in multiple replicates (at least two for each analyte on each surface). Data were double blanked by subtracting the blank in-line control surface (Spot A) and buffer reference injections. Kinetic binding data were globally fit to the Langmuir model for 1:1 binding for each replicate of concentration series using the Sierra Analyser software (version 3.4.1, Bruker Daltonics, Billerica, MA). The representative replicate data and fit were exported and then plotted using GraphPad Prism version 9.4.1. The average KD from these fits is reported along with the standard error (five replicates for acetyl-CoA and four for CoA).

Position and Linker Screen. Assays were done in 96 well plates with 100 μL final well volumes. All sensors and control proteins were diluted to 10 μM in protein storage buffer (50 mM Tris pH 7.5, 150 mM NaCl, 10% glycerol). Acetyl-CoA was dissolved in water at a concentration of 10 mM. A 5× assay buffer was used (1 μM Tris pH 7.5, 750 mM NaCl). Wells were set up in triplicate with 60 μL water, 20 μL reaction buffer, and 10 μL sensor. Then either 10 μL of water or 10 μL of acetyl-CoA were added to 1 μM sensor before reading. Plates were read at excitation 485 nm emission 528 nm. Triplicates were averaged and percent difference between no acetyl-CoA and acetyl-CoA wells were calculated.

Acetyl-CoA and CoA Titration and Spectrum Scans. Assays were done in 384 well plates with 30 μL final well volume. All sensors and control proteins were diluted to 6 μM in protein storage buffer. Acetyl-CoA and CoA were dissolved in water at a concentration of 60 UM then serial diluted to 30 μM, 15 μM, 6 μM, 3 μM, 0.6 μM, 0.06 μM, and 0.006 μM. Wells were set up in triplicate with 14 μL water, 6 μL assay buffer, and 5 μL sensor. 5 μL of an acetyl-CoA stock or water were added to 1 μM sensor before reading. For Titration curves, plates were read at excitation 485 nm and emission 514 nm. For excitation scans plates were excited from 400 nm to 500 nm at 1 nm increments and emissions were read at 428 nm. For emission scans plates were excited at 485 nm, 427 nm, or 405 nm and emissions were read from 500 nm to 575 nm in 1 nm increments. Triplicates were averaged and plotted for scans and percent difference between no acetyl-CoA and acetyl-CoA wells were calculated for titrations.

pH Titration. Assays were done in 384 well plates with 30 μL final volume. All sensors and control proteins were diluted to 6 μM in protein storage buffer. Acetyl-CoA was dissolved in water at a concentration of 6 mM. Different assay buffers were made, all 1M Tris, 750 mM NaCl with pH 6.5, 6.75, 7.0, 7.25, 7.5, 7.75, and 8.0. Wells were set up in triplicate with 14 μL water, 6 μL assay buffer, and 5 μL sensor. 5 μL of acetyl-CoA or water were added to 1 UM sensor before reading. Plates were read at excitation 485 nm and emission 514 nm. Triplicates were averaged and plotted.

Acyl-CoA Screen. Assays were done in 384 well plates with 30 μL final volume. All sensors and control proteins were diluted to 6 μM in protein storage buffer. CoA, acetyl-CoA, propionyl-CoA, butyryl-CoA, malonyl-CoA, and succinyl-CoA were dissolved in water at a concentration of 6 mM. Wells were set up in triplicate with 14 μL water, 6 μL assay buffer, and 5 μL sensor. 5 μL of an acyl-CoA or water were added to 1 μM sensor before reading. Plates were read at excitation 485 nM and emission 514 nm. Triplicates were averaged and percent difference between acyl-CoAs and water were calculated.

Flow Cytometry Starvation. All assays were done with the BD FACSAria™ III Cell Sorter. All sensors and control proteins were expressed in Rosetta (DE3) cells. PanZ was used as a non-fluorescent control, cpGFP was used as a fluorescent control, and PANcACe was the experimental. Cultures were grown in LB with kanamycin overnight at 37° C. then used to induce new cultures. Cells were grown at 37° C. to an OD600 of 0.6 then induced with 0.5 mM IPTG and grown overnight at 18° C. The overnight culture was divided into 5, 1 mL aliquots for fed, 15 minutes, 1 hour, 2 hours, and 3 hours starved timepoints. Aliquots were spun down for 5 minutes at 4000 g. Fed, 15 minutes, 1 hour, and 2 hours cells were resuspended in feeding media (i.e. PBS+28 mM glucose) while 3 hours cells were resuspended in starvation media (i.e. PBS). All cultures were put at 37° C. to shake between steps. At 2 hours, 1 hour, and 15 minutes before reading, cells were spun down as before and resuspended in starvation media then returned to shaking. On the flow cytometer cells were read with 405 nm and 485 nm excitations with 514 nm emission.

Flow Cytometry Refeeding. All assays were done with the BD FACSAria™ III Cell Sorter. All sensors and control proteins were expressed in Rosetta (DE3) cells. PanZ was used as a non-fluorescent control, cpGFP was used as a fluorescent control, and PANcACe was the experimental. Cultures were grown in LB with kanamycin overnight at 37° C. then used to induce new cultures. Cells were grown at 37° C. to an OD600 of 0.6 then induced with 0.5 mM IPTG and grown overnight at 18° C. The overnight culture was divided into 5, 1 mL aliquots for starved, 15 minutes, 1 hour, 2 hours, and 3 hours refeeding timepoints. Aliquots were spun down for 5 minutes at 4000 g. Cells were resuspended in starvation media for 3 hours before being moved into feeding media for the duration of their time point. Cells waiting to be starved were kept in feeding media. Cells were shaken at 37° C. between steps. On the flow cytometer cells were read with 405 nm and 485 nm excitations with 514 nm emission.

Flow Cytometry Data Analysis. Analysis was done in Flowing Software (Turku Bioscience, Turku, Finland). First a PanZ histogram was used as a negative control to determine how much signal came from non-fluorescent cells. The region with more fluorescence than PanZ was marked as Region 1. For PANcACe and cpGFP the ratio of 485 excitation/405 excitation was calculated on a cell-by-cell basis. This ratio was plotted in a histogram including only cells from region 1. The median of this ratio for each time point was taken. For the starvation assays the percent difference was calculated between each starvation timepoint and the fed control. For the refeeding assay the percent difference was calculated between each refeeding timepoint and the starved control.

Plate Reader Refeeding. All assays were done with the BioTek Synergy™ Plate Reader with injectors. All sensors and control proteins were expressed in Rosetta (DE3) cells. PanZ was used as a non-fluorescent control, cpGFP was used as a fluorescent control, and PANcACe was the experimental. Reads were done with excitation 405 nm and 485 nm and emission at 514 nm. Cultures were grown in LB with kanamycin overnight at 37° C. then used to induce new cultures. Cells were grown at 37° C. to an OD600 of 0.6 then induced with 0.5 mM IPTG and grown overnight at 18° C. Cultures were split into 2, 600 μL aliquots and spun at 4000 rpm for 5 minutes.

Glucose Starvation and Refeeding. The supernatant was discarded and pellets were resuspended in 600 UL either starvation buffer (100 mM NaPO₄, 2 mM MgCl₂, 15 mM (NH₄)₂SO₄+0 mM glucose) or refed buffer (100 mM NaPO₄, 2 mM MgCl₂, 15 mM (NH₄)₂SO₄+28 mM glucose). 85 μL was added to 3 wells of a 96 well plate for each sample. Plates were shaken at room temperature for 3 hours. Plates were read for 10 minutes every 30 seconds with 10 seconds of shaking between reads. 15 μL starvation buffer was added to fed cells and 15 μL of refeeding buffer (100 mM NaPO₄, 2 mM MgCl₂, 15 mM (NH₄)₂SO₄+280 mM glucose) was added to starved cells via injectors. Plates were read for 60 minutes every 30 seconds with 10 seconds of shaking between reads.

Acetate Starvation and Refeeding. The supernatant was discarded and pellets were resuspended in 600 μL either starvation buffer (100 mM NaPO₄, 2 mM MgCl₂, 15 mM (NH₄)₂SO₄+0 mM glucose) or fed buffer (100 mM NaPO₄, 2 mM MgCl₂, 15 mM (NH₄)₂SO₄+28 mM glucose). 85 μL was added to 3 wells of a 96 well plate for each sample. Plates were shaken at room temperature for 3 hours. Plates were read for 10 minutes every 30 seconds with 10 seconds of shaking between reads. 15 μL starvation buffer was added to fed cells and 15 μL of refeeding buffer (100 mM NaPO₄, 2 mM MgCl₂, 15 mM (NH₄)₂SO₄+100 μM acetate) was added to starved cells via injectors. Plates were read for 60 minutes every 30 seconds with 10 seconds of shaking between reads.

2-Deoxyglucose Starvation and Refeeding. The supernatant was discarded and pellets were resuspended in 600 μL either starvation buffer (100 mM NaPO₄, 2 mM MgCl₂, 15 mM (NH₄)₂SO₄+28 mM 2-deoxyglucose) or refeeding buffer (100 mM NaPO₄, 2 mM MgCl₂, 15 mM (NH₄)₂SO₄+28 mM glucose). 85 μL was added to 3 wells of a 96 well plate for each sample. Plates were shaken at room temperature for 3 hours. Plates were read for 10 minutes every 30 seconds with 10 seconds of shaking between reads. 15 L starvation buffer was added to fed cells and 15 μL refeeding buffer was added to starved cells via injectors. Plates were read for 60 minutes every 30 seconds with 10 seconds of shaking between reads.

Human cell handling and preparation of the stable Hela cell lines. The 293T and Hela cells were maintained in standard DMEM with 4.5 g/L glucose (11995-065, Gibco, Waltham, MA), 10% v/v heat inactivated FBS (10082-147, Gibco, Waltham, MA), and 1% v/v penicillin/streptomycin (15070063, Gibco, Waltham, MA) at 37° C. and 5% CO₂. The cells were subcultured by trypsinization (25200-056, Gibco, Waltham, MA).

Lentivirus. 293T cells were used to produce lentivirus from the pLJM1 proviral vector containing the PancAce or cpGFP gene with the corresponding localization tags. For each construct, a 10-cm dish of 293T cells in standard media with 3% v/v FBS was transfected with 4 μg provirus plasmid (pLJM1 with gene of interest), 4 μg HIV gag-pol plasmid (psPAX2), and 0.57 μg VSV-G plasmid (pMD2.G) using 26 μL of X-tremeGENE™ HP (6366244001, Sigma Millipore, St. Louis, MO) in 1 mL of Opti-MEM™ (11058-021, Gibco, Waltham, MA). After 24 h, the cells were recovered into 10 mL of standard media with 3% v/v FBS. After 48 h, the virus-containing media was harvested and replaced with 10 mL of standard media with 3% v/v FBS. After 72 h, the virus-containing media was harvested and combined with the media collected at 48 h. The virus was filtered through 0.45 μm to remove cell debris and was stored unconcentrated in 1 mL aliquots at −80° C. The Hela cells were transduced in 6-well plates with 1 mL of unconcentrated virus+10 μg/mL polybrene (TR-1003-G, Sigma Millipore, St. Louis, MO) per well for 24 h. At 48 hours post-transduction, the cells were subjected to 1 μg/mL puromycin (J67236.XF, ThermoFisher, Waltham, MA) to select for infected cells, and the selection was maintained until all the non-transduced HeLa cells were dead (about 72 h). Low passage frozen stocks were prepared of the six resultant polyclonal HeLa cell lines (PancAce mito, nuc, cyto and cpGFP mito, nuc, cyto) in 10% DMSO-containing standard media. For experiments, cells were thawed and passaged for a maximum of 4-5 weeks as sensor/GFP expression and cell viability tended to be compromised after that period.

Perturbations of the Hela cells. The PancAce/cpGFP-expressing HeLa cells were counted and plated into glass bottom 96-well plates suitable for confocal microscopy (P96-1.5H-N, CellVis). The control condition (Fed or non-targeting siRNA) in each experiment was performed in triplicate wells, and the experimental conditions were performed in singlet wells. Each experiment was performed at least 4 times on entirely different days and plates. Immediately before beginning a microscopy session, the media was changed to serum and phenol red free based media that otherwise matched the contents of the corresponding experimental condition.

Deprivation experiments. DMEM containing no glucose, phenol red, or FBS (A14430-01, Gibco, Waltham, MA) plus 4 mM glutamine (Gibco, Waltham, MA, 25030081) was used as the base deprivation media. For the Fed condition, 4.5 g/L glucose (A24940-01, Gibco, Waltham, MA) and 10% v/v FBS was added to the base media. For the (−) glucose condition, only 10% v/v FBS was added. For the dFBS condition, 4.5 g/L glucose and 10% v/v dFBS (A33820-01, Gibco, Waltham, MA) was added. For the dFBS/(−) glucose condition, only 10% v/v dFBS was added. The cells were placed in these media formulations for approximately 16 h.

Refeeding experiments. The cells were deprived of glucose as described above in DMEM with 4 mM glutamine and 10% FBS but without glucose or phenol red. After 16 h, the media was changed to DMEM with 4 mM glutamine and 4.5 g/L glucose but without phenol red or FBS (to prepare the cells for imaging). Images were collected at the indicated time points.

Branch chain amino acid (BCAA) deprivation. Powdered DMEM without glucose, glutamine, isoleucine, leucine, valine, sodium pyruvate, sodium bicarbonate, or phenol red was used as the basis for these media formulations (US Biological, Salem, MA, D9800-36). This powdered DMEM was reconstituted in water using sodium bicarbonate to adjust the pH and sterile filtered prior to use as described by the manufacturer. The media was supplemented with 4.5 g/L glucose, 4 mM glutamine, 0.8 mM leucine, and 10% FBS to generate the BCAA-deprived media (i.e., no isoleucine or valine). The control media was generated in the same way, but 0.8 mM isoleucine and 0.8 mM valine were also added. Cells were incubated in either the (+) BCAA or (−) BCAA media for 24 h.

siRNA experiments. siRNAs were purchased as SMARTPool™ from Horizon (Dharmacon, Lafayette, CO). The siRNAs were reconstituted as instructed by the manufacturer in 1×siRNA buffer (Horizon, Waterbeach, United Kingdom) in RNase-free water to a concentration of 20 μM. These stock aliquots were stored at −20° C. Each well of a 96-well plate was transfected with 0.3 μL of Lipofectamine 3000 (L3000001 ThermoFisher, Waltham, MA) and 50 nM siRNA (final concentration) in 300 μL total of Opti-MEM™. After incubation for 5-6 hours, the cells were recovered by changing the media to standard DMEM with 10% FBS and 1% pen/strep. The knockdowns were validated by immunoblot as detailed in the Immunoblotting section below. The siRNAs and antibodies are listed in TABLE 2 and TABLE 3.

TABLE 2
SMARTPool ™ siRNAs

| siRNA | Catalog ID |
|---|---|
| Control: ON-TARGETplus Non-targeting Pool | D-001810-10 |
| ON-TARGETplus Human ACLY siRNA | L-004915-00-0005 |
| ON-TARGETplus Human PDHA1 siRNA | L-010329-00-0005 |
| ON-TARGETplus Human ACSS2 siRNA | L-010396-00-0005 |
| ON-TARGETplus Human CRAT siRNA | L-009524-00-0005 |
| ON-TARGETplus Human EP300 siRNA | L-003486-00-0005 |
| ON-TARGETplus Human ACACA siRNA | L-004551-00-0005 |
| ON-TARGETplus Human ACACB siRNA | L-004759-00-0005 |

TABLE 3
Antibodies

| Antibody | Manufacturer | Catalog ID |
|---|---|---|
| Mouse anti-actin mAb | Cell Signaling Technologies (Danvers, MA) | 3700 |
| Rabbit anti-ACLY mAb | Cell Signaling Technologies (Danvers, MA) | 13390 |
| Rabbit anti-ACSS2 mAb | Cell Signaling Technologies (Danvers, MA) | 3658 |
| Rabbit anti-PDH mAb | Cell Signaling Technologies (Danvers, MA) | 3205 |
| Rabbit anti-CrAT pAb | Proteintech ® | 15170-1-AP |
| Rabbit anti-p300 mAb | Cell Signaling Technologies (Danvers, MA) | 57625 |
| Rabbit anti-ACC mAb | Cell Signaling Technologies (Danvers, MA) | 3676 |

The cells were imaged 48 hours after transfection. Immediately before beginning the microscopy session, the media was changed to phenol red and serum free media containing 4.5 g/L glucose and 4 mM glutamine.

Fluorescence microscopy data collection. The cells were maintained at 37° C. and 5% CO₂ during imaging using an Oko Lab (Pozzuoli N A, Italy) stage top incubator. A Leica SP8 White Light Confocal microscope (Leica Microsystems, Wetzlar, Germany) was used for imaging. A Leica 20× dry objective was used (Leica Microsystems, Wetzlar, Germany). Excitation was performed at 405 nm and 488 nm, and emission was measured at 515 nm. Both excitation lasers were operated at 6% power. The gain of the PMT detector varied depending on the overall brightness of the cells on a given day such that pixel saturation was avoided. The scan parameters were set to 200x scan speed, 2.00 zoom factor, 3.5 μm pinhole. For each well, 10 FOVs were collected encompassing about 100 cells per FOV.

Fluorescence microscopy data analysis. The images were processed using Fiji (ImageJ, National Institutes of Health). A custom Python script was used for batch processing. Each pair of 405 nm and 488 nm images was subjected to a background subtraction (sliding rolling ball 50 px width) and a gaussian blur of 2, and then the ratio image was generated by dividing the 488 nm/405 nm. A mask for the ratio image was prepared by summing the 405 nm and 488 nm images that had a background subtraction and a gaussian blur applied as above. This mask was then applied to the ratio image. The mean pixel density was measured for the masked ratio image using an Otsu threshold. For the 10 FOVs per well, outliers were first removed using an, and an outlier were defined as a FOV that was more than three standard deviations outside the mean of the 10 FOVs. Then the total pixel area of the final processed ratio images was calculated and used to weigh the average mean pixel density of the 10 FOVs (minus any outliers). This value was taken as the measurement for each well. Next, the PancAce measurement was divided by the corresponding cpGFP measurement for a given condition (i.e., PancAce/cpGFP). For visualizing data, it was further normalized by dividing the experimental by the corresponding control condition (e.g., Fed/deprived) to give a fold change. For some plots, the percentage change is displayed from the following calculation: −(1−(foldchange))*100. The statistical analyses are described in detail in the Statistics section below.

Immunoblotting. Hela cell samples were treated as described in the section above for nutrient deprivation or siRNA transfections. The only difference was that conditions for immunoblotting were scaled up to a 6-well plate format. The cells were washed with cold PBS and then harvested in cold PBS. The Epiquik™ Histone Extraction Kit (Epigentek, Farmingdale, NY) was used for lysis and histone extraction as directed by the manufacturer. Briefly, the cell pellets were resuspended in 100 μL of 1× Pre-Lysis buffer and lysed on ice for 10 minutes. The cells were pelleted at high speed for 1 minute, and the supernatant was reserved (“lysate” or “total soluble fraction”). The pellet was resuspended in 30 μL of the Lysis buffer and incubated on ice for 30 minutes. The material was pelleted, and the supernatant was reserved (“histones”) and neutralized with 9 μL of Balance buffer as directed. SDS loading buffer was added to both lysate and histone samples for immunoblotting.

The protein samples were separated by SDS-PAGE gel (either 12% Bis Tris or 4-12% Bis Tris as indicated for each blot) and transferred to a PVDF membrane by semi-dry transfer in Towbin buffer. The membranes were blocked with 3% or 5% milk according to the antibody manufacturer's instructions for 1 hour at room temperature. The membranes were incubated with primary antibodies at 4° C. overnight and then washed with TBST 3×. The membranes were incubated with secondary antibodies (Licor) in TBST for 1 hour at room temperature and then washed with TBST 3× before imaging using an Odyssey® Imager. Densitometry was performed in the LI-COR software (LI-COR Biosciences, Lincoln, NE).

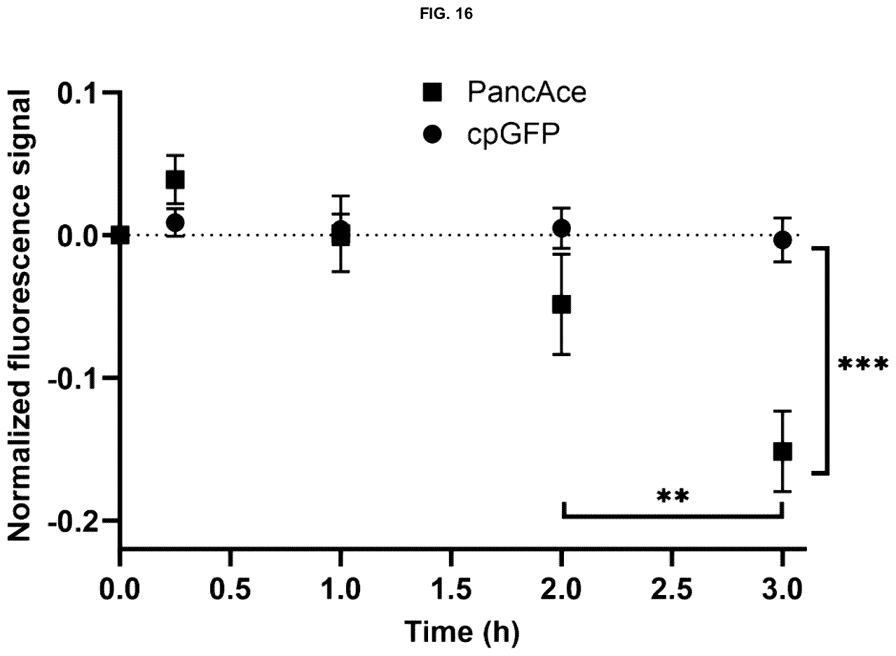

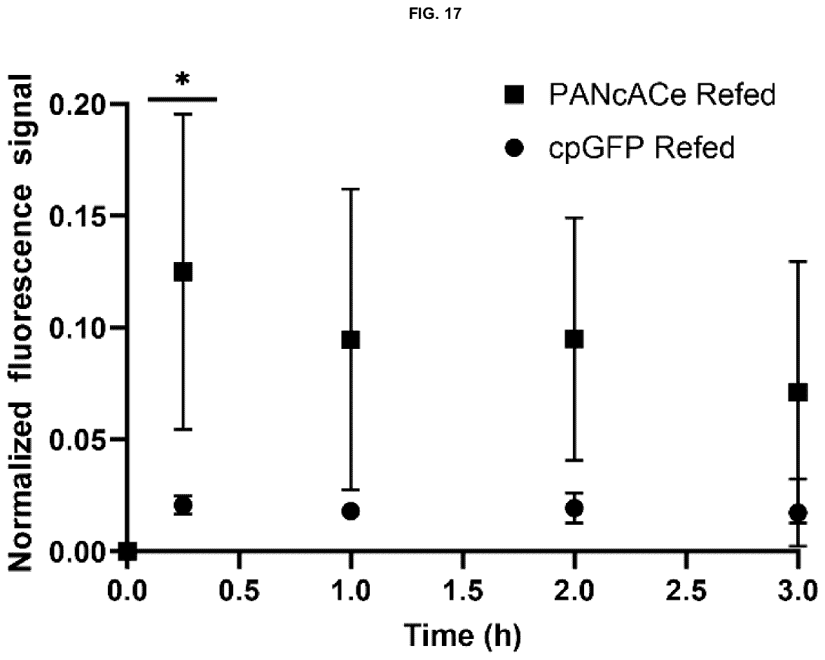

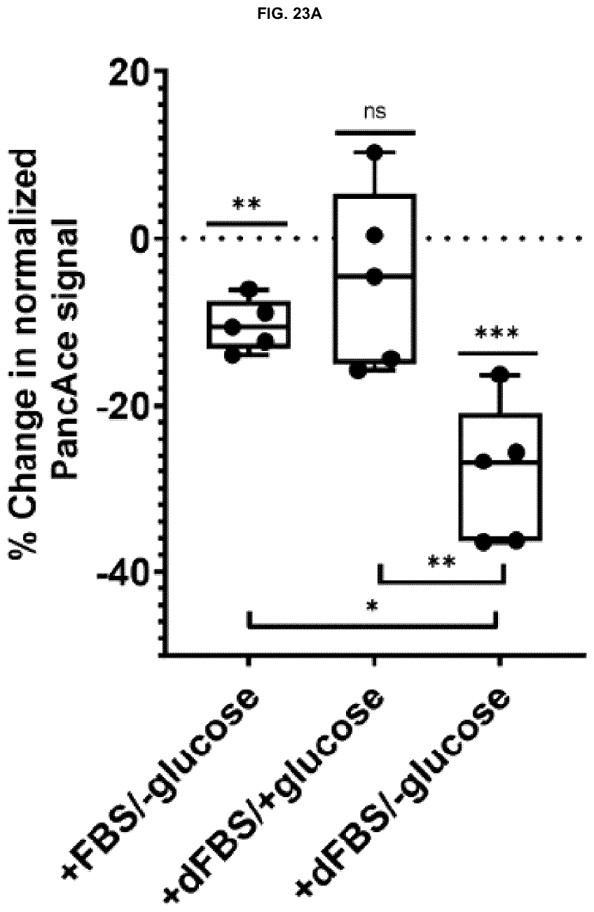

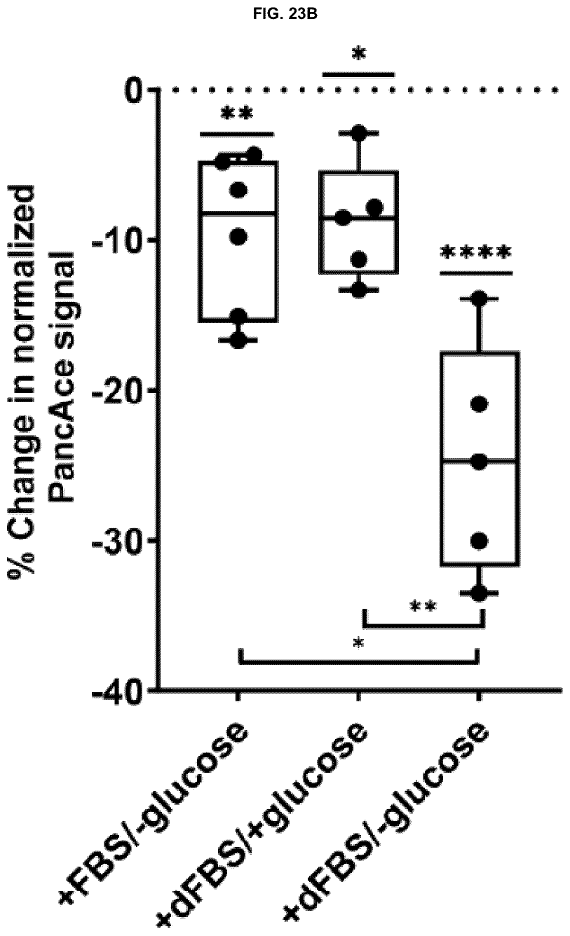

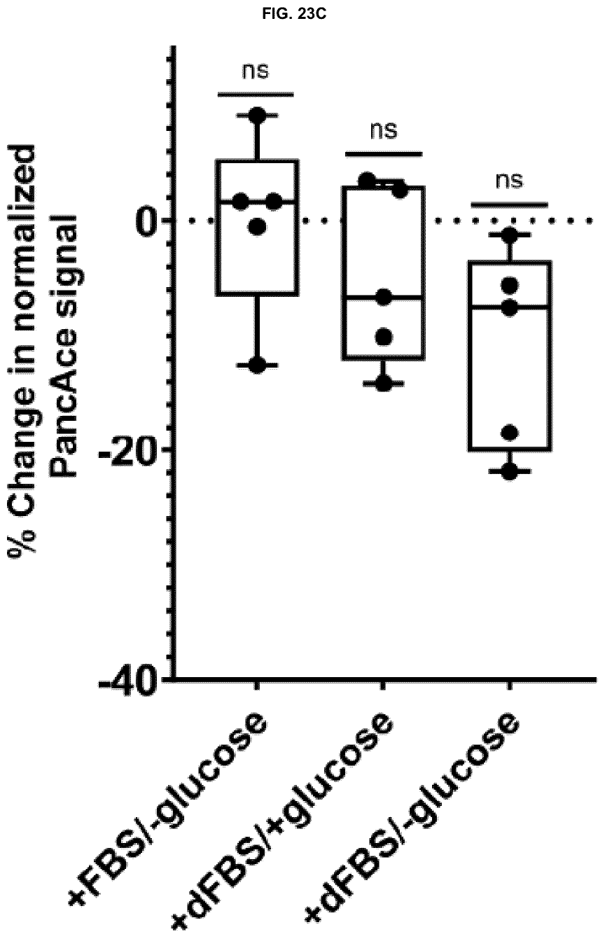

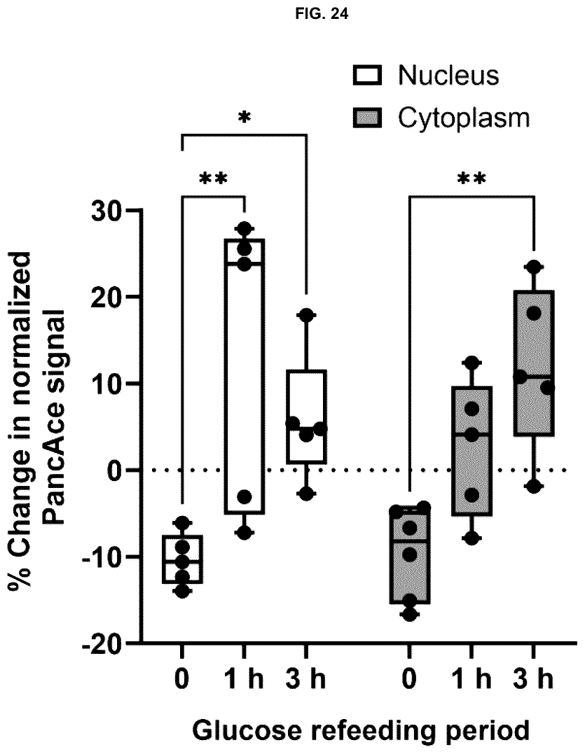

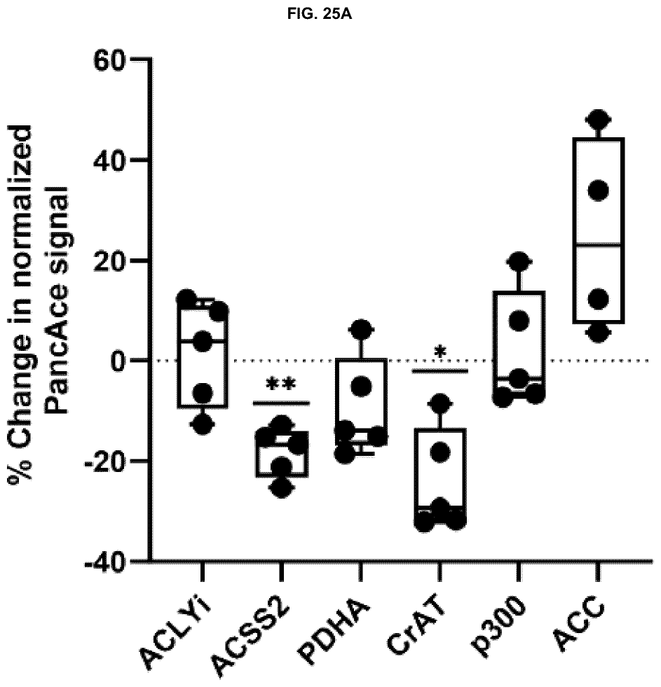

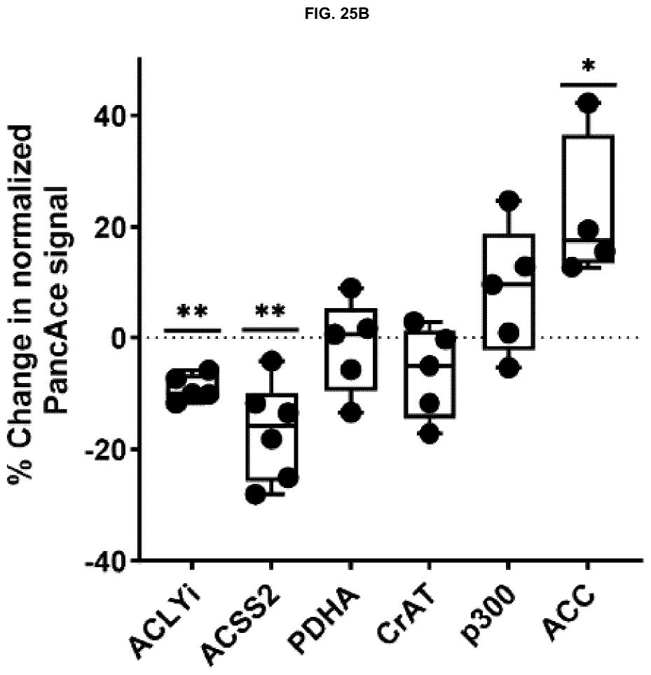

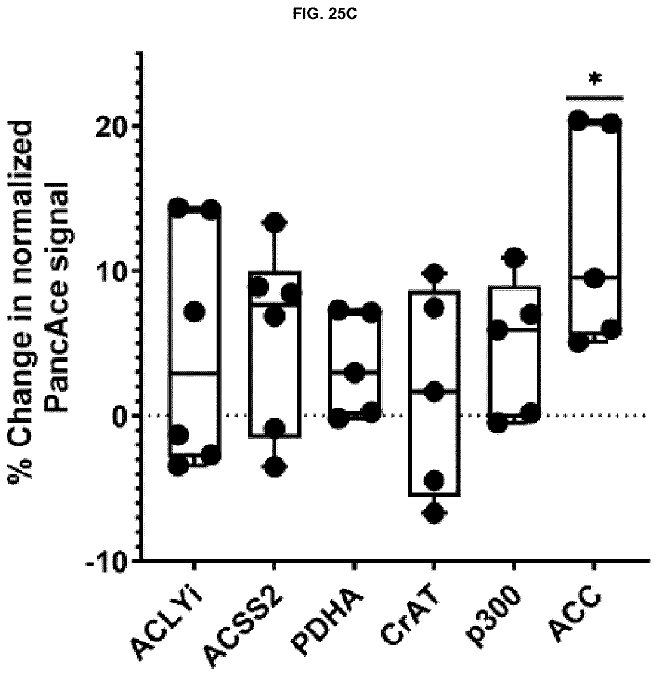

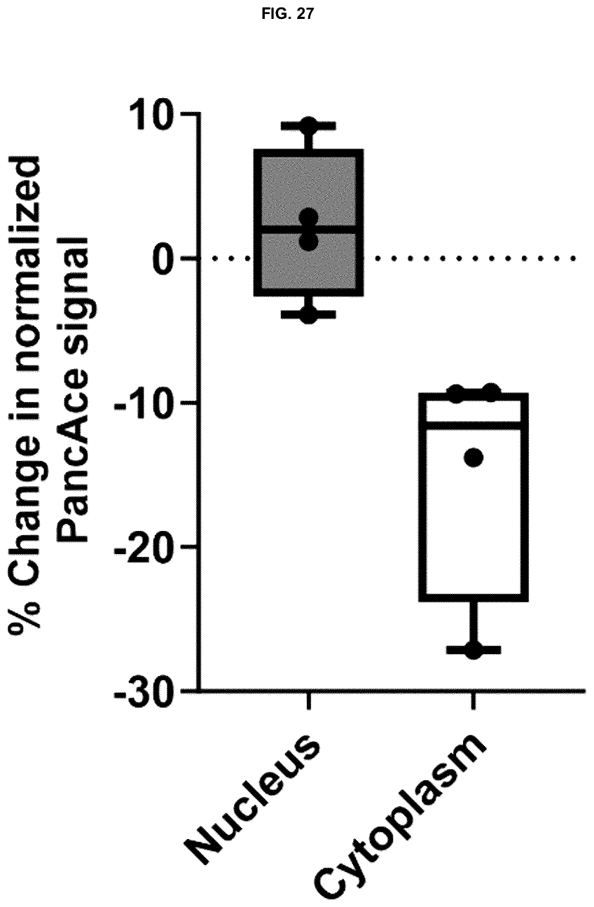

Statistics. Prism Graphpad (version 10) was used for statistical analysis. A one-way ANOVA was applied to the data in FIG. 16 and FIG. 17. A two-way ANOVA was applied to the non-normalized data in FIG. 23A, FIG. 23B, FIG. 23C, FIG. 24, FIG. 25A, FIG. 25B, FIG. 25C, and FIG. 27. These tests yielded p-values, and each plot caption indicates the p-value ranges. The error bars shown in the plots and text are the standard deviations.

### Example 2

### Biosensor Engineering

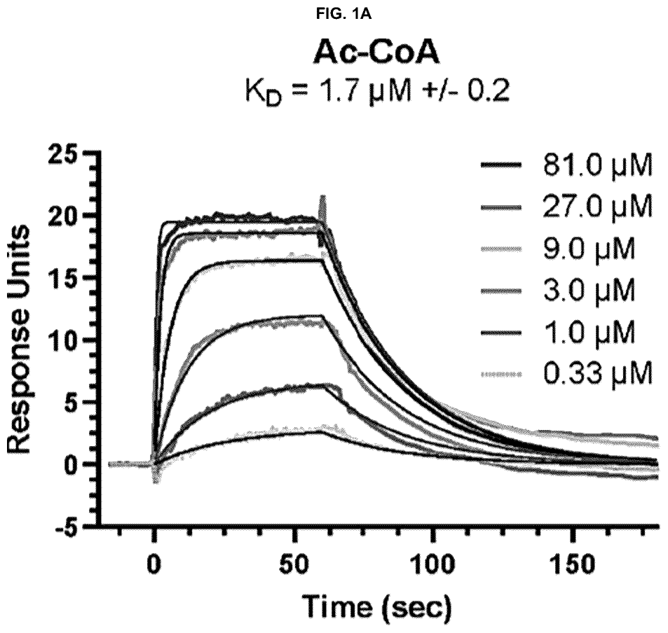

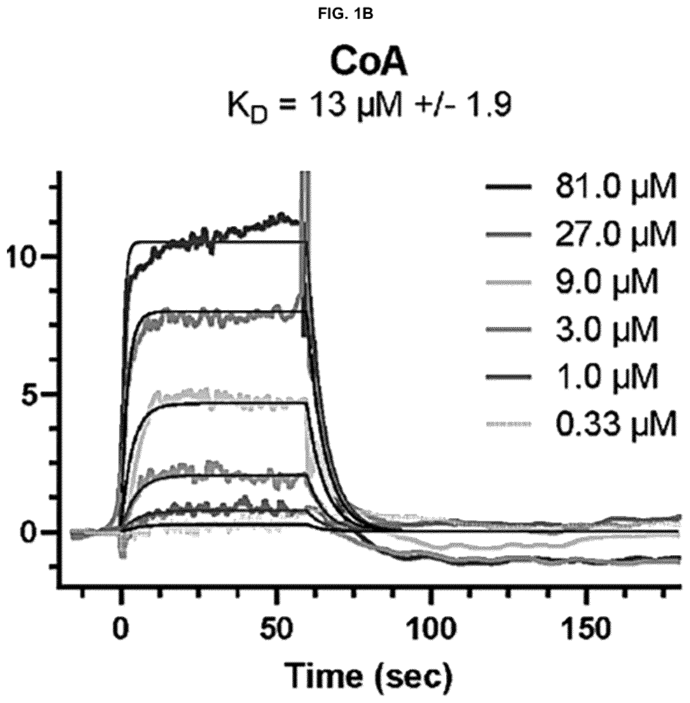

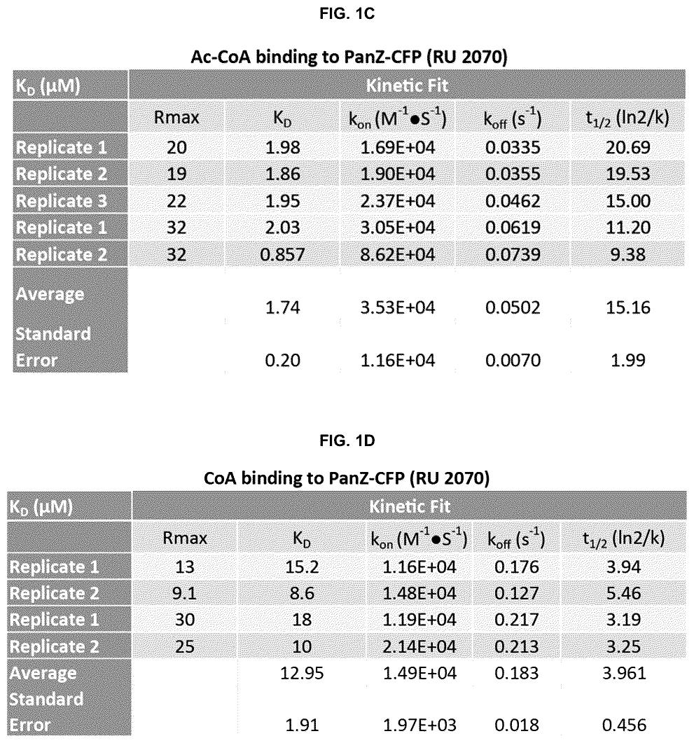

First, an acetyl-CoA binding protein was selected that was predicted to be amenable to forming a biosensor. The protein PanZ was chosen, which is an endogenous acetyl-CoA sensor in E. coli that participates in the regulation of pantothenate synthesis. PanZ has a GNAT (GCN5-related N-acetyltransferase) domain but was shown to lack acetyltransferase activity. Instead of acting as an enzyme itself, PanZ binds acetyl-CoA, and this bound state is competent to bind the zymogen PanD (and to the activated form, aspartate α-decarboxylase). A structure of the PanZ/acetyl-CoA/PanD complex (PDB: 5LS7) and other biochemical data suggested that PanZ might be a suitable basis for a fluorescent acetyl-CoA sensor. However, one concern was that previous data was unclear as to the selectivity of PanZ for acetyl-CoA versus CoA. Poor selectivity would limit the utility of a biosensor for directly measuring acetyl-CoA in cells where acetyl-CoA and CoA levels are comparable under some conditions. The binding of purified PanZ to acetyl-CoA and CoA was tested using surface plasmon resonance (SPR) and measured a 7-fold higher affinity for acetyl-CoA versus CoA (FIGS. 1A-1D). The K_{d} for acetyl-CoA agreed with the previously published value. Based on this data, it was concluded that PanZ was a promising starting point for a biosensor.

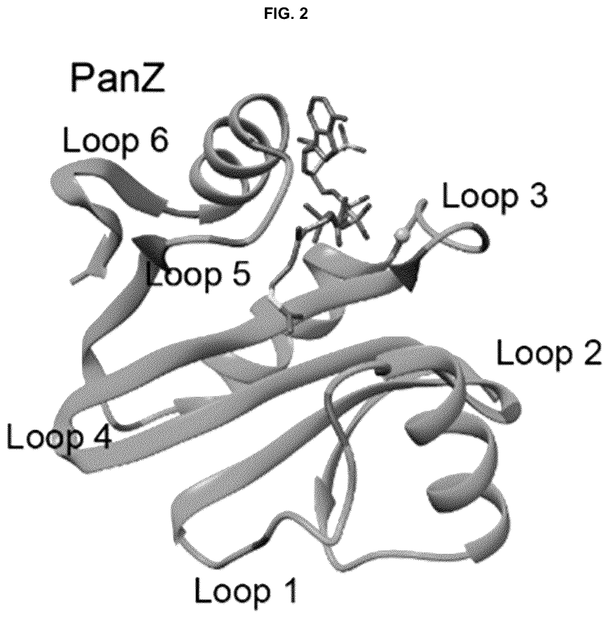

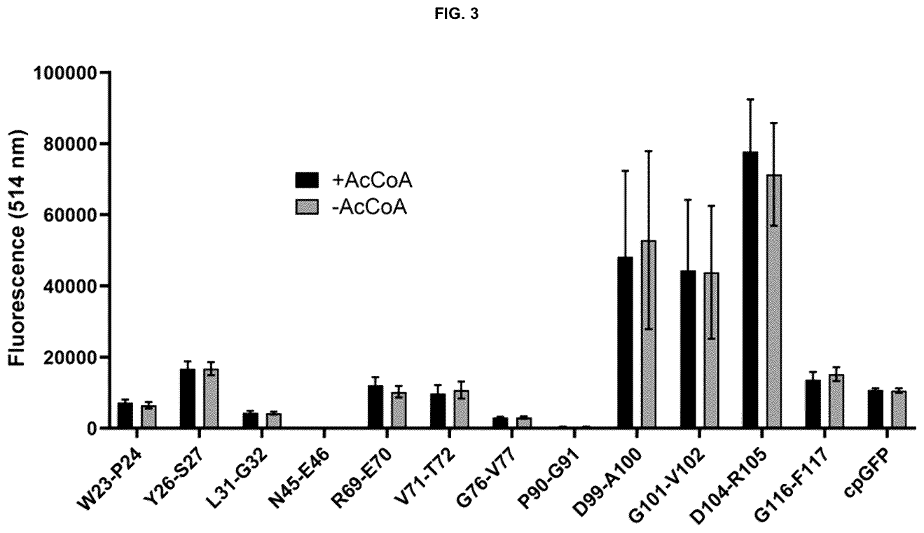

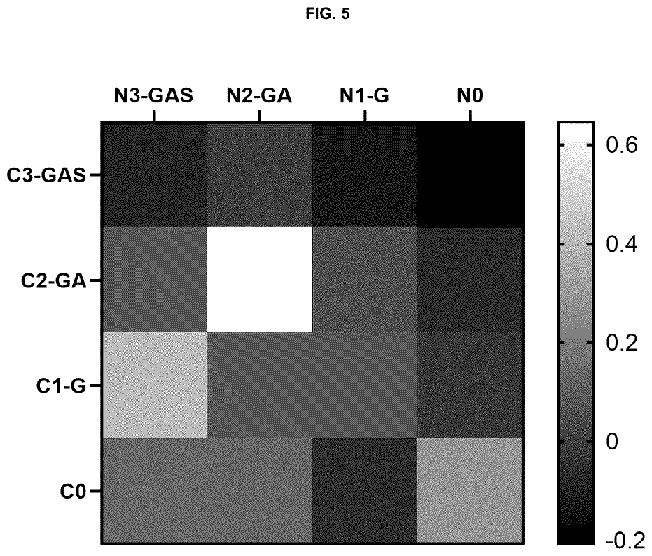

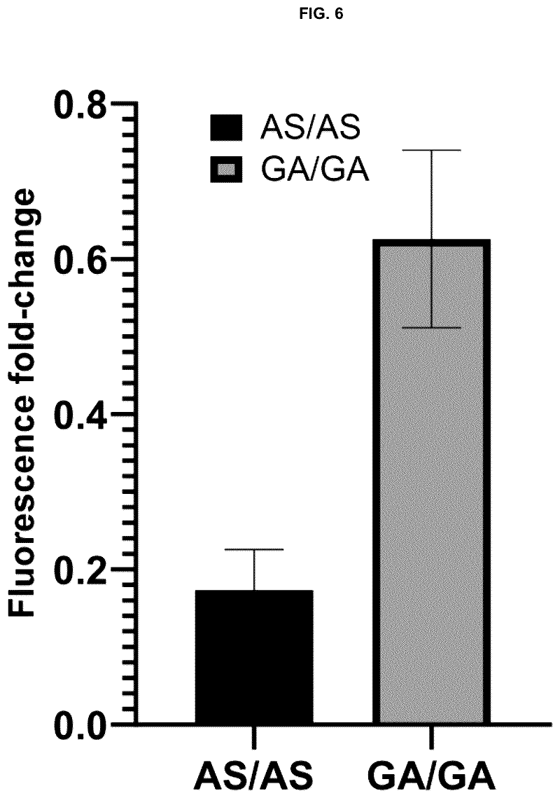

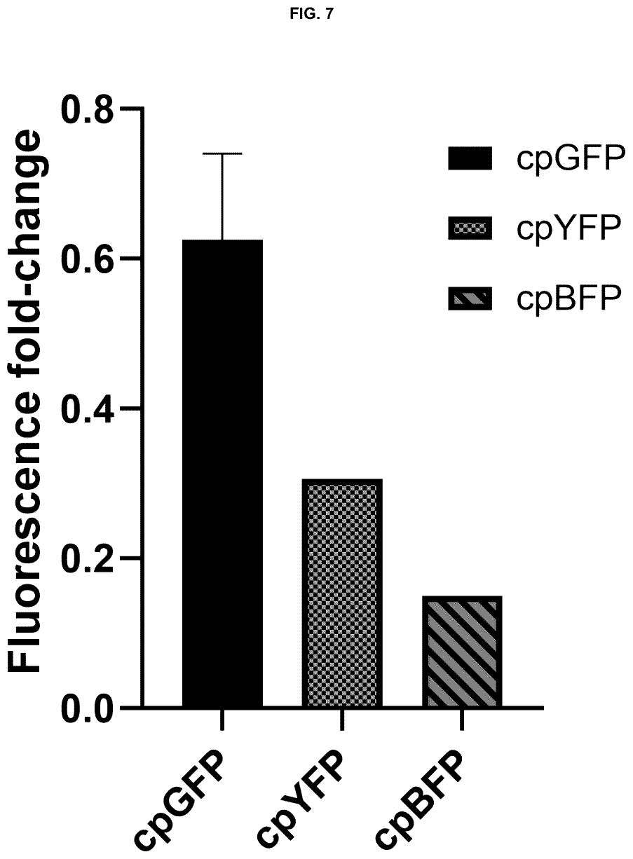

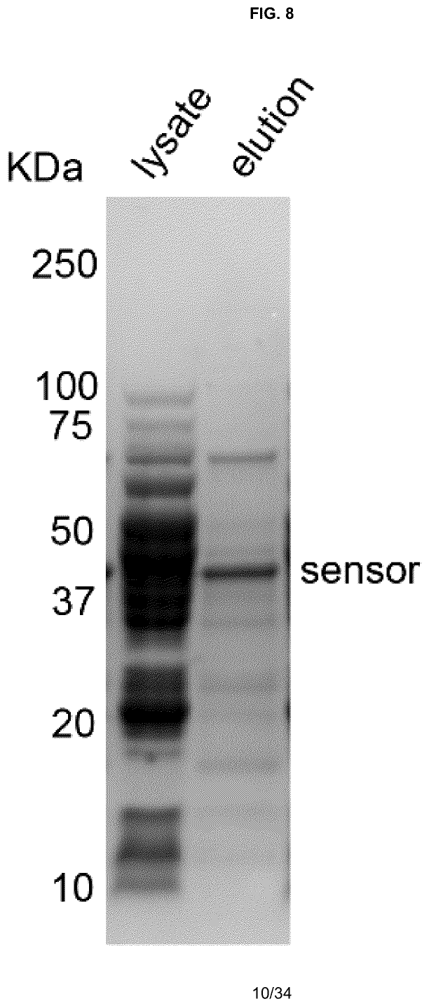

To engineer a fluorescent sensor, insertion sites of circularly permuted GFP (cpGFP) in loop regions of PanZ (FIG. 2) were screened (FIG. 3 and FIG. 4). The variants ranged widely in terms of fluorescence intensity and yield (FIG. 3 and FIG. 4). Considering the fold-change between the +/−acetyl-CoA conditions, the overall fluorescence intensity, and the yield of each variant, the R69-E70 insertion site was selected as the best-performing from the panel. Next, a linker length screen based on this R69-E70 variant was performed. The original R69-E70 construct contained an AS linker on either side of the cpGFP (i.e., Nterm-PanZ-AS-cpGFP-AS-PanZ-Cterm). The linker combinations shown were purified and tested, and the GA linker at both the N-terminus and C-terminus of cpGFP was revealed to perform best (FIG. 5) and was even better than the original AS linker by about 2.5-fold in terms of response (FIG. 6). This version of the biosensor is termed PancAce (pronounced “pancake”; for PanZ Acetyl-CoA sensor, FIG. 8). CpYFP and cpBFP (yellow and blue cpFPs, respectively) were tested with the PancAce variant and were found to perform as acetyl-CoA biosensors, although they have a lower response range than PancAce (FIG. 7).

### Example 3

### Biochemical Characterization of PancAce

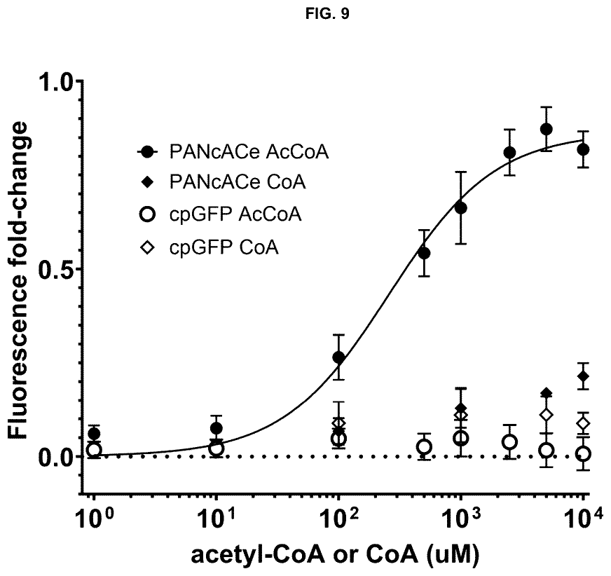

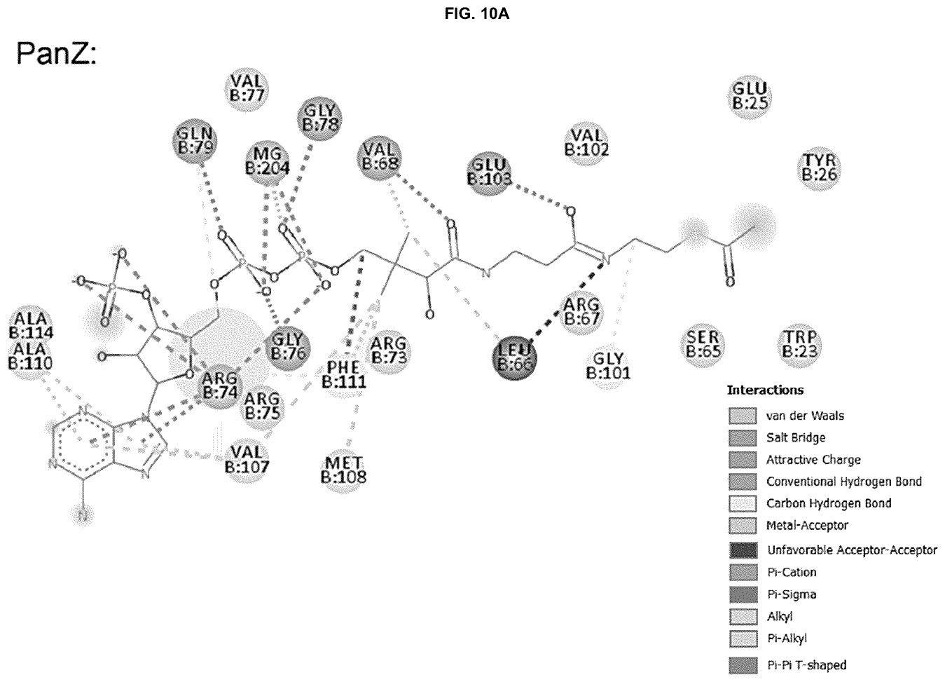

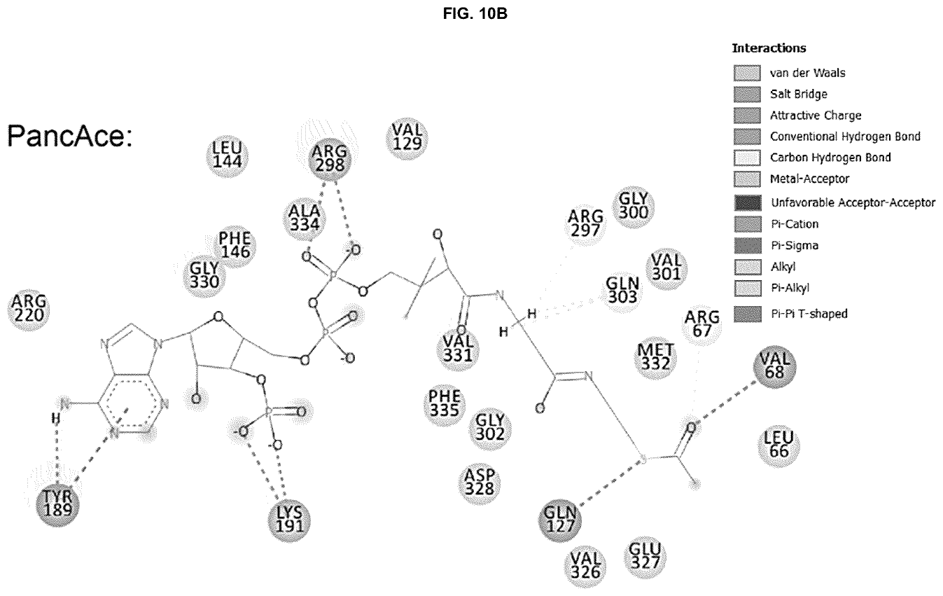

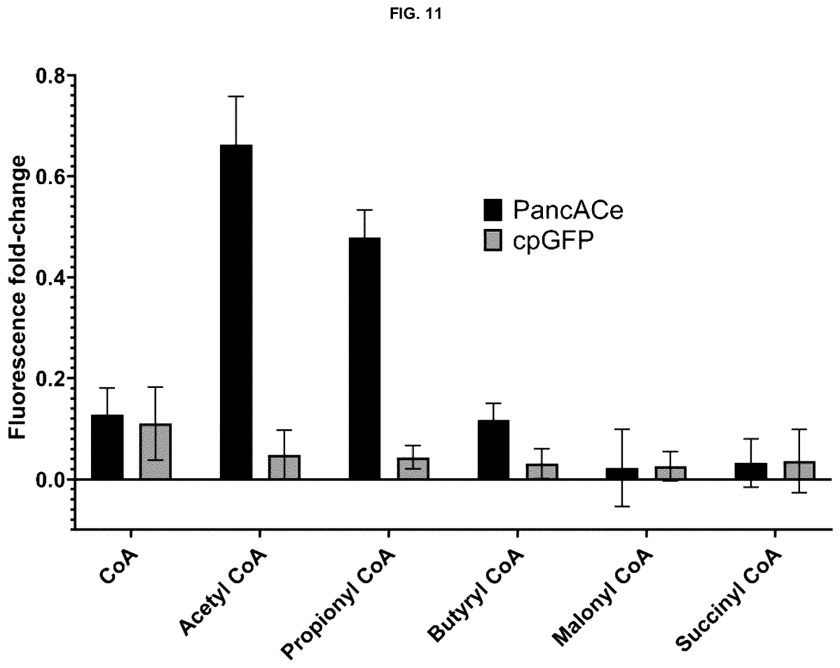

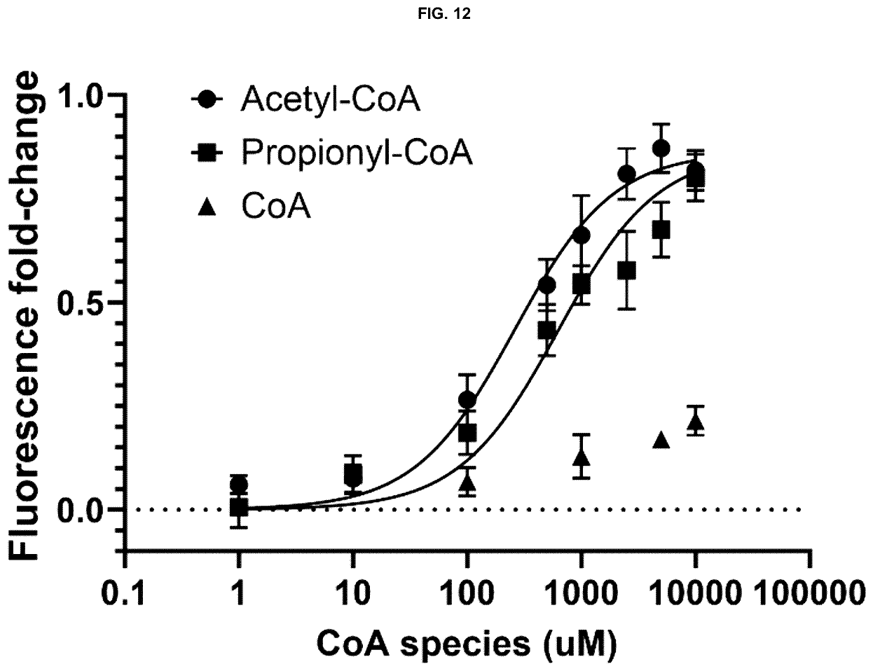

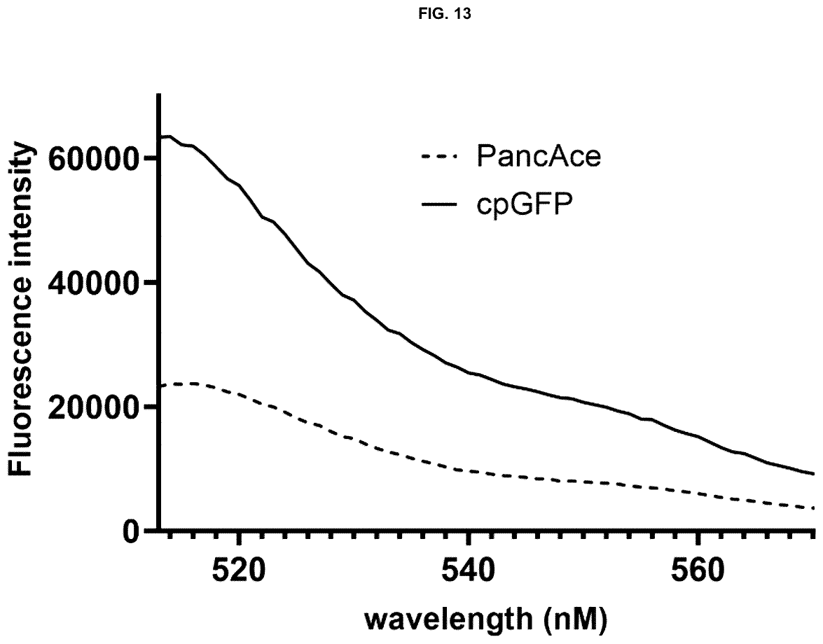

Titrations of acetyl-CoA and CoA with PANcACe and cpGFP alone were performed (FIG. 9). PANcACe displays an increase in fluorescence upon binding to acetyl-CoA and has a maximum response of almost 2-fold at saturating acetyl-CoA. This response range is comparable to many other reported metabolite biosensors derived from a cpFP. The affinity of PANcACe for acetyl-CoA (K_{d,app}=258±38 μM from fluorescence data) is about 150-fold lower compared to PanZ (K_{d}=1.7±0.2 μM from SPR data). Since the insertion site of cpGFP is very close to the acetyl-CoA binding site, this effect was not surprising. Indeed, a molecular model of the sensor shows that the cpGFP insertion alters the acetyl-CoA binding site of the PanZ significantly (FIGS. 10A-10B). PANcACe shows good selectivity for acetyl-CoA over CoA (FIG. 9), which is consistent with the SPR binding data for PanZ (7-fold, FIG. 1A). Even though the PancAce-CoA data cannot be fitted to obtain a K_{d} since saturation was not achieved, interpolation between the PancAce-AcCoA and -CoA data indicate that 10 mM CoA is required to elicit the same sensor response as 100 μM acetyl-CoA. Further, the selectivity of PANcACe for short-chain acyl-CoAs was tested (FIG. 11). The only acyl-CoA that was also recognized by the sensor was propionyl-CoA, and based on the single concentration experiment, the affinity of PancAce for propionyl-CoA was lower than for acetyl-CoA. A propionyl-CoA titration confirmed this apparent affinity difference (FIG. 12). Since saturation was not achieved up to 10 mM propionyl-CoA, it was assumed that the same maximal sensor response would be achieved for propionyl-CoA as for acetyl-CoA. Based on this assumption, the data was fit and a K_{d}=626±75 μM was estimated for propionyl-CoA, a 2.4-fold lower affinity compared to acetyl-CoA.

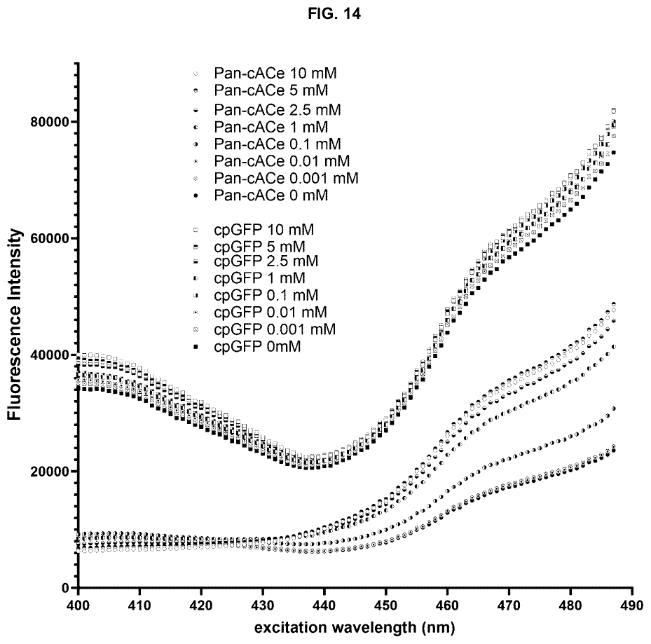

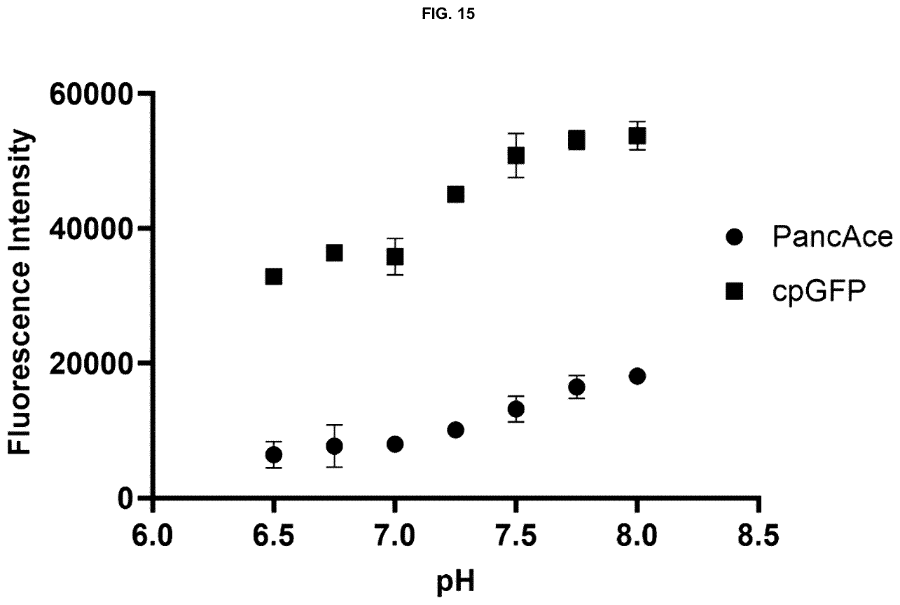

Since fluorescent proteins display pH sensitivity, the fluorescence of PANcACe and cpGFP over a range of pH values (6.5-8, FIG. 15) were measured and it was observed that there was similar behavior as has been reported previously for fluorescent proteins and biosensors constructed from them. Finally, it was determined whether the same type of internal control that was used by another group with their cpVenus (cpV)-derived NAD+biosensor could be used (Cambronne et al., Science (2016) 352, 1474-1477). Cambronne et al. found that a second excitation wavelength (λₑₓ=405 nm, λₑₘ=520 nm) of cpV was largely invariant in the presence of NAD+ and used this wavelength as an internal control for the amount of sensor present in their cell-based assays. PancAce also has this 405 nm excitation peak that is invariant to acetyl-CoA (FIG. 14) that could be used in the same way for live cell experiments.

### Example 4

Acetyl-CoA Measurements in Live E. coli

Either PancAce or cpGFP was expressed in E. coli (Rosetta DE3 cells) and their nutrient status was manipulated to measure changes in acetyl-CoA levels by measuring the sensor response via flow cytometry or well plate reader. λₑₓ=405 nm and 485 nm (both with λₑₘ=514 nm) was measured to allow for normalizing the data to account for changes due to pH, protein expression, photobleaching, or other factors that might influence the fluorescence signal that are not indicative of acetyl-CoA.

First, cells were incubated in phosphate-buffered saline (PBS) with no glucose or other nutrients for increasing periods of time. Flow cytometry was used to measure fluorescence, and the signal from each starvation time point was normalized to the signal from fed cells (PBS+28 mM glucose) expressing the corresponding construct (fed=0, FIG. 16). cpGFP-expressing cells that were treated identically to the PancAce-expressing cells were used as a control. After 2 hours of nutrient deprivation, PancAce showed a decrease in its fluorescence response while the cpGFP signal remained invariant over the entire time course. Next, a refeeding experiment was performed in which cells were first starved in PBS for 3 hours and then were refed with 28 mM glucose for different periods of time (FIG. 17). For this experiment, the time points were normalized to the signal from starved cells expressing the corresponding construct (starved=0). Within 15 minutes, PancAce displayed an increase in signal that was much greater than the small increase in the signal of cpGFP, suggesting that the cells recovered their intracellular acetyl-CoA levels rapidly upon refeeding.

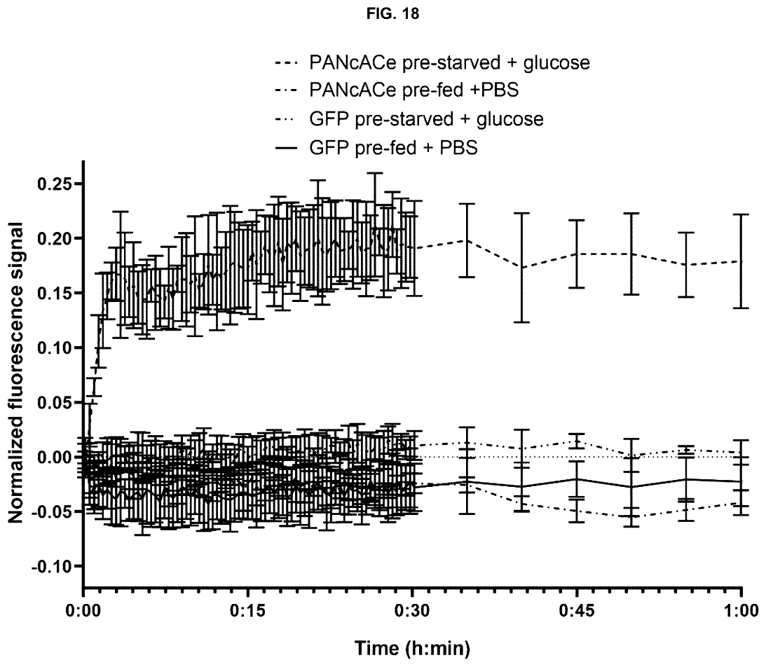

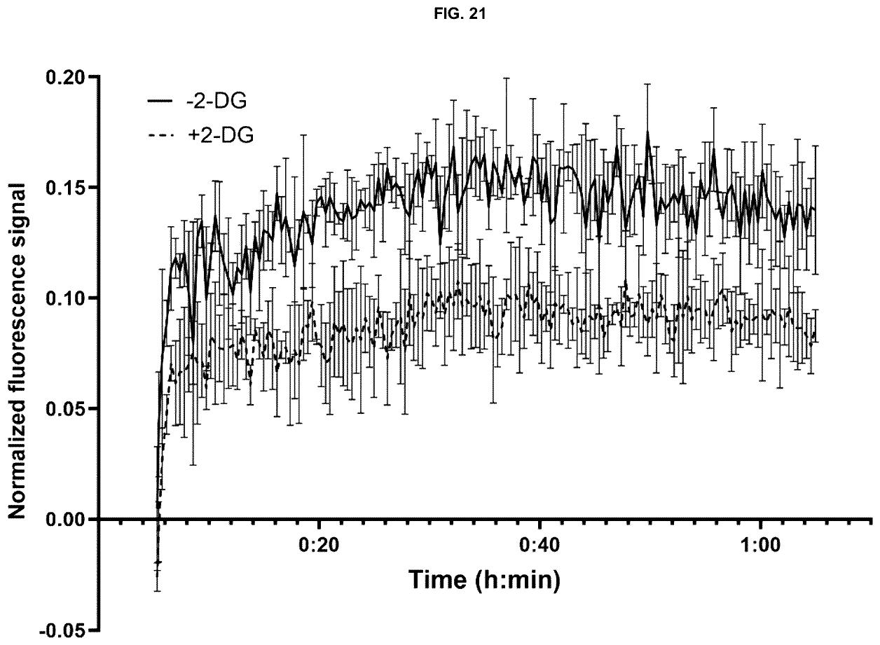

Since this change happened fast and was pushing the limits of how quickly the experimental workflow could be performed, a well plate reader was used instead to measure the fluorescence in situ so that repeated measurements of a cell population could be taken in increments of less than 1 minute. Injectors were used to rapidly add assays components. First, cells that had either been pre-starved (no glucose for 3 h) or fed (+28 mM glucose for 3 h) were used. After briefly measuring the baseline signal, 28 mM glucose was added to the starved cells or buffer without glucose to the fed cells (FIG. 18). Only the PancAce-expressing starved cells that were fed with glucose showed a sharp increase in signal, and this occurred in just a few minutes, which was consistent with our refeeding flow cytometry experiment. Next, the cells were treated with 2-deoxyglucose (2-DG), an inhibitor of glycolysis (FIG. 21). Both sets of cells were deprived of glucose for 3 hours, but one set was also given 2-DG during that time period. Then the cells were refed with 28 mM glucose, and the sensor response was monitored over 1 hour. As expected, the cells treated with 2-DG showed a lower recovery of acetyl-CoA levels upon refeeding, but they were still able to largely recover.

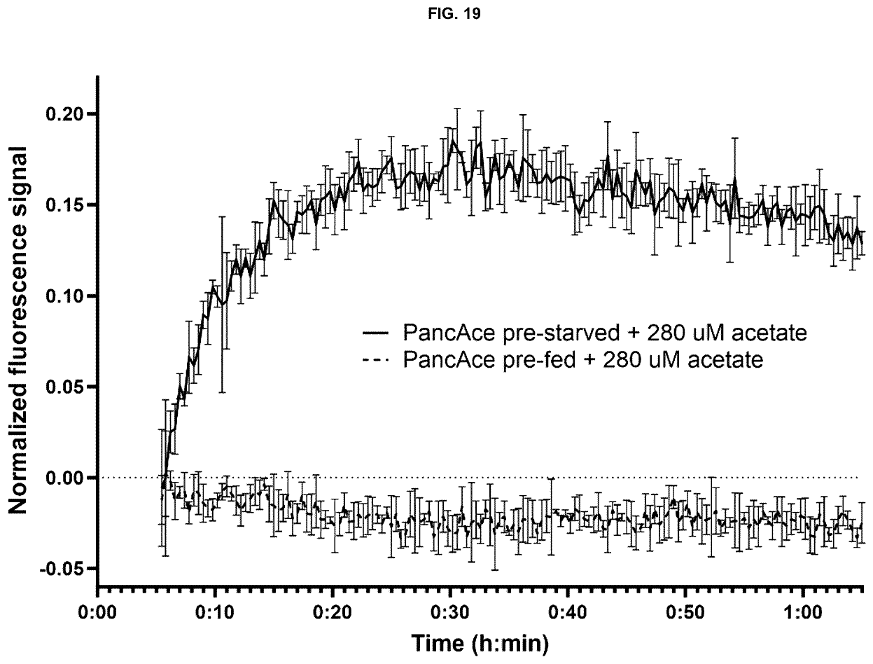

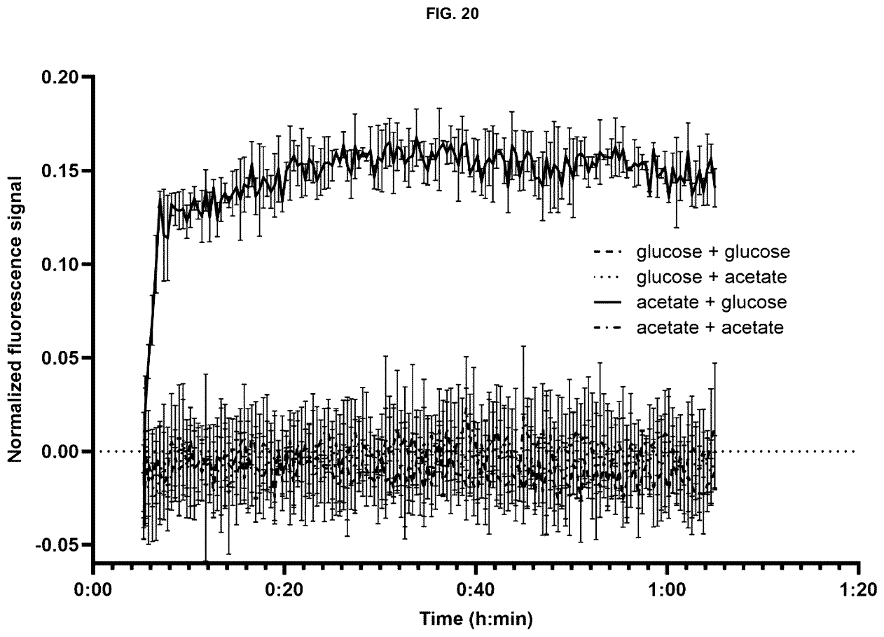

Finally, starved or glucose-fed cells were fed with acetate instead of glucose since acetate can be converted to acetyl-CoA via non-glycolytic pathways. Based on the use of 100 UM acetate for experiments in mammalian cells, 280 μM acetate was chosen. The starved cells exhibited a relatively gradual increase in PancAce signal upon acetate feeding (increase over about 30 minutes), compared to the sharp change observed with glucose feeding (FIGS. 19-20). The fed cells exhibited no change upon addition of acetate.

### Example 5

### Acetyl-CoA Measurements in Hela Cells

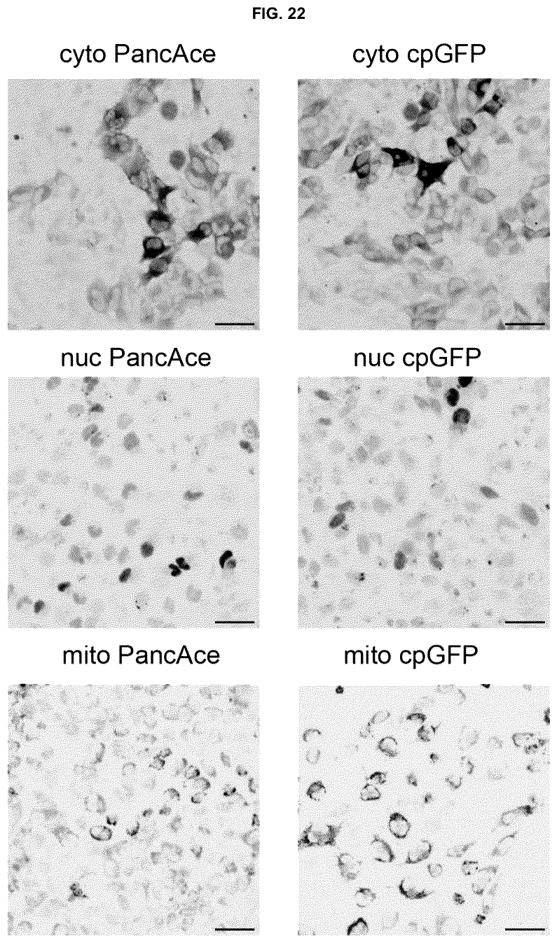

HeLa cell lines stably expressing PancAce or cpGFP with a nuclear, cytoplasmic, or mitochondrial localization tag were prepared (FIG. 22) and fluorescence microscopy was used to quantify the PancAce and cpGFP signals. To perturb intracellular acetyl-CoA levels, the cells were deprived of glucose, complete serum (i.e., cells given dialyzed fetal bovine serum, dFBS), or both for 18 hours and then compared the sensor response to fed cells given glucose and complete serum during that same period (FIGS. 23A-23C). The dFBS was used since FBS contains acetate and glucose both of which can feed into acetyl-CoA. Nuclear acetyl-CoA levels were sensitive to glucose deprivation as expected based on previous findings. Serum deprivation did not significantly affect nuclear acetyl-CoA levels, but deprivation of both nutrients had a synergistic impact. Cytoplasmic acetyl-CoA levels decreased under all of the deprivation conditions with the deprivation of both glucose and serum causing a larger decrease than either deprivation alone. Mitochondrial acetyl-CoA levels did not change according to the PancAce sensor.

Next, how long it would take for acetyl-CoA levels to recover in cells that were glucose deprived and then refed with glucose was analyzed (FIG. 24). It was observed that the nucleus acetyl-CoA levels were at least partially restored after 1 hour and fully restored after 3 hours. Cytoplasmic acetyl-CoA levels were restored after 3 hours and were trending up after only 1 hour.

Since PancAce only has a 2.4-fold lower affinity for propionyl-CoA, how sensitive the sensor was in responding to changes in propionyl-CoA instead of acetyl-CoA was evaluated. Based on previous work that showed that branched chain amino acid (BCAA) deprivation lowered nuclear propionyl-CoA levels much more than acetyl-CoA levels (Trefely, et al., Mol. Cell (2022) 82, 447-462), this perturbation with PancACe was used. In Trefely, et al, cells deprived of BCAAs showed a reduction in both acetyl-CoA and propionyl-CoA in the non-nuclear fraction of the cells, while the nucleus showed a reduction in propionyl-CoA but not acetyl-CoA. Cells were either given complete media or starved of BCAAs (valine and isoleucine) for 24 h (FIG. 27). The nuclear compartment showed no significant change in the sensor response, while the cytoplasm displayed a decrease in the PancAce signal. Based on this data and the data from Trefely, et al, PancAce appears to be selective for acetyl-CoA over propionyl-CoA in cells.

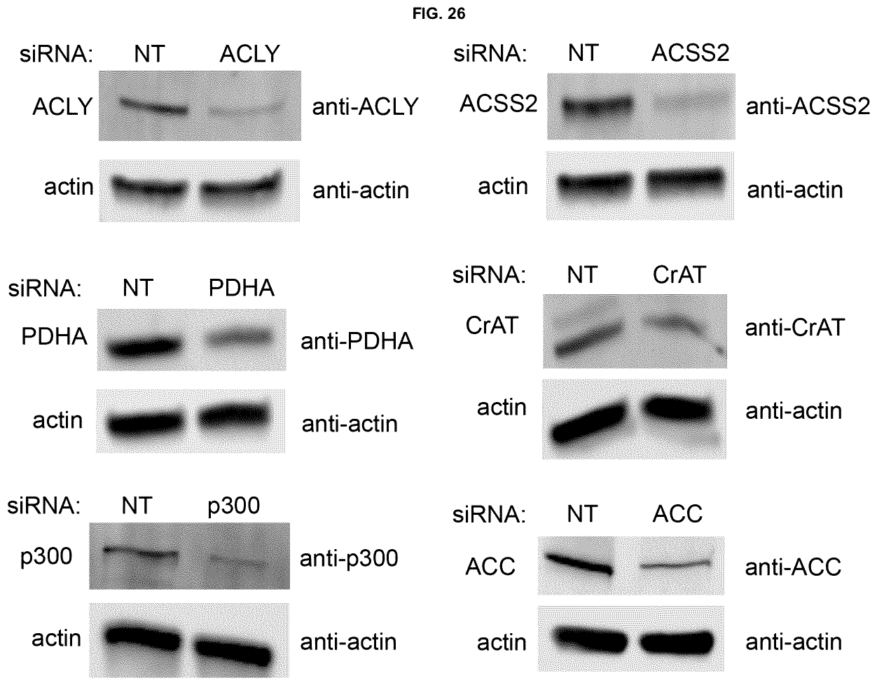

Acetyl-CoA is produced and consumed by disparate pathways in the cell in a compartmentalized fashion. Thus, a panel of proteins was knocked-down (FIG. 26) that modulate acetyl-CoA by producing, transporting, or consuming acetyl-CoA and PancAce was used to measure compart-specific changes in acetyl-CoA levels (FIGS. 25A-25C). The data for the nuclear-localized PancAce shows that acyl-CoA short chain synthetase 2 (ACSS2) and carnitine acetyltransferase (CrAT) are primarily responsible for producing acetyl-CoA there, while ACLY (ATP citrate lyase) and PDHA (pyruvate dehydrogenase, isoform A) do not measurably contribute to nuclear acetyl-CoA. Knockdown of the acetyltransferase p300 did not result in accumulation of acetyl-CoA in the nucleus, but knockdown of acetyl-CoA carboxylase (ACC, isoforms A and B) did cause accumulation of acetyl-CoA in the nucleus. In the cytoplasm, the data indicates that ACLY and ACSS2 are the major contributors to acetyl-CoA. Like in the nucleus, ACC knockdown causes accumulation of cytoplasmic acetyl-CoA, as expected. No major changes occurred to acetyl-CoA in the mitochondria except, when ACC was knocked down, there was accumulation of acetyl-CoA like in the other compartments. These data show that PancAce can measure acetyl-CoA levels in different cell compartments and be used to characterize the compartment-specific effects of modulating metabolic enzymes.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A recombinant acetyl-coenzyme A (acetyl-CoA) biosensor polypeptide comprising: an acetyl-CoA binding protein having an amino acid sequence at least 95% identical to SEQ ID NO: 1, wherein the acetyl-CoA binding protein is divided into: a first acetyl-CoA binding protein fragment comprising an N-terminal portion of the acetyl-CoA binding protein; and a second acetyl-CoA binding protein fragment comprising a C-terminal portion of the acetyl-CoA binding protein; wherein the first and second acetyl-CoA binding protein fragments collectively include all of the amino acids of the acetyl-CoA binding protein; and a fluorescent protein inserted between the first and second acetyl-CoA binding protein fragments and attached to a C-terminus of the first acetyl-CoA binding protein fragment and an N-terminus of the second acetyl-CoA binding protein fragment; and wherein: (i) the C-terminus is an arginine at position 69 of SEQ ID NO: 1 (Arg69) and the N-terminus is a glutamic acid at position 70 of SEQ ID NO: 1 (Glu70); (ii) the C-terminus is a tryptophan at position 23 of SEQ ID NO: 1 (Trp23) and the N-terminus is a proline at position 24 of SEQ ID NO: 1 (Pro24); (iii) the C-terminus is a valine at position 71 of SEQ ID NO: 1 (Val71) and the N-terminus is a threonine at position 72 of SEQ ID NO: 1 (Thr72); (iv) the C-terminus is an aspartic acid at position 99 of SEQ ID NO: 1 (Asp99) and the N-terminus is an alanine at position 100 of SEQ ID NO: 1 (Ala100); (v) the C-terminus is an aspartic acid at 104 of SEQ ID NO: 1 (Asp104) and the N-terminus is an arginine at position 105 of SEQ ID NO: 1 (Arg105); or (vi) the C-terminus is a glycine at position 116 of SEQ ID NO: 1 (Gly116) and the N-terminus is a phenylalanine at position 117 of SEQ ID NO: 1 (Phe117); and wherein the recombinant acetyl-CoA biosensor polypeptide selectively binds acetyl-CoA, and the binding of acetyl-CoA induces a change in the fluorescence of the fluorescent protein.

Clause 2. The recombinant acetyl-CoA biosensor polypeptide of clause 1, wherein the fluorescent protein is a circularly permuted GFP (cpGFP), a circularly permuted yellow fluorescent protein (cpYFP), or a circularly permuted blue fluorescent protein (cpBFP).

Clause 3. The recombinant acetyl-CoA biosensor polypeptide of clause 2, wherein the cpGFP comprises an amino acid sequence of SEQ ID NO: 2, the cpYFP comprises an amino acid sequence of SEQ ID NO: 3, and the cpBFP comprises an amino acid sequence of SEQ ID NO: 4.

Clause 4. The recombinant acetyl-CoA biosensor polypeptide of any one of clauses 1-3, wherein the acetyl-CoA binding protein comprises an amino acid sequence at least 99% identical to SEQ ID NO: 1.

Clause 5. The recombinant acetyl-CoA biosensor polypeptide of any one of clauses 1-4, wherein the acetyl-CoA binding protein comprises the amino acid sequence of SEQ ID NO: 1.

Clause 6. The recombinant acetyl-CoA biosensor polypeptide of any one of clauses 1-5, wherein: the fluorescent protein is either directly attached to the C-terminus of the first acetyl-CoA binding protein fragment or is attached by a first amino acid linker that is from 1 to 3 amino acids in length; and the fluorescent protein is either directly attached to the N-terminus of the second acetyl-CoA binding protein fragment or is attached by a second linker that is from 1 to 3 amino acids in length.

Clause 7. The recombinant acetyl-CoA biosensor polypeptide of clause 6, wherein the first and second amino acid linkers are each independently selected from the group consisting of a Gly, Gly-Ala, Ala-Ser, and Gly-Ala-Ser.

Clause 8. The recombinant acetyl-CoA biosensor polypeptide of clause 6 or clause 7, wherein: (i) the first linker is Gly-Ala and the second linker is Gly-Ala; (ii) the first linker is Ala-Ser and the second linker is Ala-Ser; (iii) the first linker is Gly-Ala-Ser and the second linker is Gly; (iv) the C-terminus and N-terminus are directly attached to the fluorescent protein; (v) the C-terminus is directly attached to the fluorescent protein and the second linker is Gly-Ala-Ser; (vi) the first linker is Gly-Ala-Ser and the N-terminus is directly attached to the fluorescent protein; (vii) the first linker is Gly-Ala and the N-terminus is directly attached to the fluorescent protein; or (viii) the first linker is Gly and the second linker is Gly-Ala-Ser.

Clause 9. The recombinant acetyl-CoA biosensor polypeptide of any one of clauses 1-8, further comprising one or more of a histidine tag, a TEV cleavage site, a FLAG® tag, a human influenza hemagglutinin (HA) tag, a nuclear export signal, a nuclear localization signal, a cytoplasmic localization signal, and a mitochondrial localization signal at the N-terminal portion of the acetyl-CoA binding protein.

Clause 10. The recombinant acetyl-CoA biosensor polypeptide of any one of clauses 1-9, wherein: (i) the recombinant acetyl-CoA biosensor polypeptide comprises the amino acid sequence of SEQ ID NO: 5; (ii) the recombinant acetyl-CoA biosensor polypeptide comprises the amino acid sequence of SEQ ID NO: 6; (iii) the recombinant acetyl-CoA biosensor polypeptide comprises the amino acid sequence of SEQ ID NO: 7; (iv) the recombinant acetyl-CoA biosensor polypeptide comprises the amino acid sequence of SEQ ID NO: 8; (v) the recombinant acetyl-CoA biosensor polypeptide comprises the amino acid sequence of SEQ ID NO: 9; (vi) the recombinant acetyl-CoA biosensor polypeptide comprises the amino acid sequence of SEQ ID NO: 10; (vii) the recombinant acetyl-CoA biosensor polypeptide comprises the amino acid sequence of SEQ ID NO: 11; (viii) the recombinant acetyl-CoA biosensor polypeptide comprises the amino acid sequence of SEQ ID NO: 12; (ix) the recombinant acetyl-CoA biosensor polypeptide comprises the amino acid sequence of SEQ ID NO: 13; (x) the recombinant acetyl-CoA biosensor polypeptide comprises the amino acid sequence of SEQ ID NO: 14; (xi) the recombinant acetyl-CoA biosensor polypeptide comprises the amino acid sequence of SEQ ID NO: 15; (xii) the recombinant acetyl-CoA biosensor polypeptide comprises the amino acid sequence of SEQ ID NO: 16; (xiii) the recombinant acetyl-CoA biosensor polypeptide comprises the amino acid sequence of SEQ ID NO: 17; (xiv) the recombinant acetyl-CoA biosensor polypeptide comprises the amino acid sequence of SEQ ID NO: 18; or (xv) the recombinant acetyl-CoA biosensor polypeptide comprises the amino acid sequence of SEQ ID NO: 19.

Clause 11. The recombinant acetyl-CoA biosensor polypeptide of clause 10, wherein the recombinant acetyl-CoA biosensor polypeptide comprises the amino acid of SEQ ID NO: 5.

Clause 12. An expression vector comprising: a nucleic acid that encodes the recombinant acetyl-CoA biosensor polypeptide of any one of clauses 1-11; and a promoter operably linked to the nucleic acid.

Clause 13. The expression vector of clause 12, wherein the expression vector is a lentiviral vector, an adeno-associated virus (AAV) vector, or a cytomegalovirus (CMV) vector.

Clause 14. A method of detecting acetyl-CoA in a sample comprising: contacting the sample with the recombinant acetyl-CoA biosensor polypeptide of any one of clauses 1-11; exciting the recombinant acetyl-CoA biosensor polypeptide in the sample at an excitation wavelength; measuring a fluorescence intensity of the recombinant acetyl-CoA biosensor polypeptide in the sample at an emission wavelength; and comparing the fluorescence intensity to a standard curve, wherein the fluorescence intensity correlates with a concentration of acetyl-CoA in the sample.

Clause 15. The method of clause 14, wherein the excitation wavelength is from about 460 nm to about 490 nm.

Clause 16. The method of clause 14 or clause 15, wherein the excitation wavelength is 485 nm.

Clause 17. The method of any one of clauses 14-16, wherein the emission wavelength is from about 513 nm to about 540 nm.

Clause 18. The method of any one of clauses 14-17, wherein the emission wavelength is 514 nm.

Clause 19. The method of any one of clauses 14-18, wherein the pH of the sample is maintained at a pH of 6.5-8.0.

Clause 20. A method of monitoring acetyl-CoA activity in a cell, comprising: providing a cell with the recombinant acetyl-CoA biosensor polypeptide of any one of clauses 1-11; exciting the recombinant acetyl-CoA biosensor polypeptide in the cell at a first excitation wavelength between about 400 nm and about 430 nm while measuring a first fluorescence intensity at an emission wavelength between about 513 nm and about 540 nm; exciting the recombinant acetyl-CoA biosensor polypeptide in the cell at a second excitation wavelength between about 460 nm and about 490 nm while measuring a second fluorescence intensity at the emission wavelength; and normalizing the second fluorescence intensity based on the first fluorescence intensity.

Clause 21. The method of clause 20, wherein normalizing comprises dividing the second fluorescence intensity by the first fluorescence intensity.

Clause 22. The method of clause 20 or clause 21, further comprising treating the cell with an acetyl-CoA precursor or nutrient affecting the function of the cell and comparing the normalized fluorescence intensity of the cell to the normalized fluorescence intensity of a control cell.

Clause 23. The method of clause 22, wherein one or more of a nuclear export signal, a nuclear localization signal, a cytoplasmic localization signal, and a mitochondrial localization signal is attached to an N-terminus of the recombinant acetyl-CoA biosensor polypeptide.

Clause 24. The method of clause 23, further comprising determining where acetyl-CoA is localized in the cell.

Clause 25. The method of any one of clauses 20-24, wherein the first excitation wavelength is 405 nm.

Clause 26. The method of any one of clauses 20-25, wherein the second excitation wavelength is 485 nm.

Clause 27. The method of any one of clauses 20-26, wherein the emission wavelength is 514 nm.

Clause 28. The method of any one of clauses 20-27, wherein the providing step comprises transforming the cell with a plasmid comprising a polynucleotide that encodes the recombinant acetyl-CoA biosensor polypeptide.

SEQUENCES
PanZ Amino Acid Sequence
SEQ ID NO: 1
MKLTIIRLEKFSDQDRIDLQKIWPEYSPSSLQVDDNHRIYAARFNERLLAAVRVTLSGTEGALDSLRVRE
VTRRRGVGQYLLEEVLRNNPGVSCWWMADAGVEDRGVMTAFMQALGFTAQQGGWEKC
cpGFP Amino Acid Sequence
SEQ ID NO: 2
NVYIKADKQKNGIKANFKIRHNIEDGGVQLAYHYQQNTPIGDGPVLLPDNHYLSVQSKLSKDPNEKRDHM
VLLEFVTAAGITLGMDELYKGGTGGSMVSKGEELFTGVVPILVELDGDVNGHKESVSGEGEGDATYGKLT
LKFICTTGKLPVPWPTLVTTLTYGVQCFSRYPDHMKQHDFFKSAMPEGYIQERTIFFKDDGNYKTRAEVK
FEGDTLVNRIELKGIDFKEDGNILGHKLEYN
cpYFP Amino Acid Sequence
SEQ ID NO: 3
NVYIKADKQKNGIKANFKIRHNIEDGGVQLAYHYQQNTPIGDGPVLLPDNHYLSYQSVLSKDPNEKRDHM
VLLEFVTAAGITLGMDELYKGGTGGSMVSKGEELFTGVVPILVELDGDVNGHKFSVSGEGEGDATYGKLT
LKFICTTGKLPVPWPTLVTTLTYGVQCFSRYPDHMKQHDFFKSAMPEGYIQERTIFFKDDGNYKTRAEVK
FEGDTLVNRIELKGIDFKEDGNILGHKLEYN
cpBFP Amino Acid Sequence
SEQ ID NO: 4
NVYIKADKQKNGIKANFKIRHNIEDGGVQLAYHYQQNTPIGDGPVLLPDNHYLSVQSKLSKDPNEKRDHM
VLLEFVTAAGITLGMDELYKGGTGGSMVSKGEELFTGVVPILVELDGDVNGHKESVSGEGEGDATYGKLT
LKFICTTGKLPVPWPTLVTTLSHGVQCFSRYPDHMKQHDFFKSAMPEGYIQERTIFFKDDGNYKTRAEVK
FEGDTLVNRIELKGIDFKEDGNILGHKLEYN
PanZ(R69)-GA-cpGFP-GA-(E70)PanZ (“PANcACe”) Amino Acid Sequence
SEQ ID NO: 5
MKLTIIRLEKESDQDRIDLQKIWPEYSPSSLQVDDNHRIYAARFNERLLAAVRVTLSGTEGALDSLRVRG
ANVYIKADKQKNGIKANFKIRHNIEDGGVQLAYHYQQNTPIGDGPVLLPDNHYLSVQSKLSKDPNEKRDH
MVLLEFVTAAGITLGMDELYKGGTGGSMVSKGEELFTGVVPILVELDGDVNGHKESVSGEGEGDATYGKL
TLKFICTTGKLPVPWPTLVTTLTYGVQCFSRYPDHMKQHDFFKSAMPEGYIQERTIFFKDDGNYKTRAEV
KFEGDTLVNRIELKGIDFKEDGNILGHKLEYNGAEVTRRRGVGQYLLEEVLRNNPGVSCWWMADAGVEDR
GVMTAFMQALGFTAQQGGWEKC
PanZ(W23)-AS-cpGFP-AS-(P24)PanZ Amino Acid Sequence
SEQ ID NO: 6
MKLTIIRLEKESDQDRIDLQKIWASNVYIKADKQKNGIKANFKIRHNIEDGGVQLAYHYQQNTPIGDGPV
LLPDNHYLSVQSKLSKDPNEKRDHMVLLEFVTAAGITLGMDELYKGGTGGSMVSKGEELFTGVVPILVEL
DGDVNGHKFSVSGEGEGDATYGKLTLKFICTTGKLPVPWPTLVTTLTYGVQCFSRYPDHMKQHDFFKSAM
PEGYIQERTIFFKDDGNYKTRAEVKFEGDTLVNRIELKGIDFKEDGNILGHKLEYNASPEYSPSSLQVDD
NHRIYAARFNERLLAAVRVTLSGTEGALDSLRVREVTRRRGVGQYLLEEVLRNNPGVSCWWMADAGVEDR
GVMTAFMQALGFTAQQGGWEKC
PanZ(V71)-AS-cpGFP-AS-(T72)PanZ Amino Acid Sequence
SEQ ID NO: 7
MKLTIIRLEKESDQDRIDLQKIWPEYSPSSLQVDDNHRIYAARFNERLLAAVRVTLSGTEGALDSLRVRE
VASNVYIKADKQKNGIKANFKIRHNIEDGGVQLAYHYQQNTPIGDGPVLLPDNHYLSVQSKLSKDPNEKR
DHMVLLEFVTAAGITLGMDELYKGGTGGSMVSKGEELFTGVVPILVELDGDVNGHKESVSGEGEGDATYG
KLTLKFICTTGKLPVPWPTLVTTLTYGVQCFSRYPDHMKQHDFFKSAMPEGYIQERTIFFKDDGNYKTRA
EVKFEGDTLVNRIELKGIDFKEDGNILGHKLEYNASTRRRGVGQYLLEEVLRNNPGVSCWWMADAGVEDR
GVMTAFMQALGFTAQQGGWEKC
PanZ(R69)-AS-cpGFP-AS-(E70)PanZ Amino Acid Sequence
SEQ ID NO: 8
MKLTIIRLEKFSDQDRIDLQKIWPEYSPSSLQVDDNHRIYAARFNERLLAAVRVTLSGTEGALDSLRVRA
SNVYIKADKQKNGIKANFKIRHNIEDGGVQLAYHYQQNTPIGDGPVLLPDNHYLSVQSKLSKDPNEKRDH
MVLLEFVTAAGITLGMDELYKGGTGGSMVSKGEELFTGVVPILVELDGDVNGHKESVSGEGEGDATYGKL
TLKFICTTGKLPVPWPTLVTTLTYGVQCFSRYPDHMKQHDFFKSAMPEGYIQERTIFFKDDGNYKTRAEV
KFEGDTLVNRIELKGIDFKEDGNILGHKLEYNASEVTRRRGVGQYLLEEVLRNNPGVSCWWMADAGVEDR
GVMTAFMQALGFTAQQGGWEKC
PanZ(D99)-AS-cpGFP-AS-(A100)PanZ Amino Acid Sequence
SEQ ID NO: 9
MKLTIIRLEKFSDQDRIDLQKIWPEYSPSSLQVDDNHRIYAARFNERLLAAVRVTLSGTEGALDSLRVRE
VTRRRGVGQYLLEEVLRNNPGVSCWWMADASNVYIKADKQKNGIKANFKIRHNIEDGGVQLAYHYQQNTP
IGDGPVLLPDNHYLSVQSKLSKDPNEKRDHMVLLEFVTAAGITLGMDELYKGGTGGSMVSKGEELFTGVV
PILVELDGDVNGHKFSVSGEGEGDATYGKLTLKFICTTGKLPVPWPTLVTTLTYGVQCFSRYPDHMKQHD
FFKSAMPEGYIQERTIFFKDDGNYKTRAEVKFEGDTLVNRIELKGIDFKEDGNILGHKLEYNASAGVEDR
GVMTAFMQALGFTAQQGGWEKC
PanZ(D104)-AS-cpGFP-AS-(R105)PanZ Amino Acid Sequence
SEQ ID NO: 10
MKLTIIRLEKFSDQDRIDLQKIWPEYSPSSLQVDDNHRIYAARFNERLLAAVRVTLSGTEGALDSLRVRE
VTRRRGVGQYLLEEVLRNNPGVSCWWMADAGVEDASNVYIKADKQKNGIKANFKIRHNIEDGGVQLAYHY
QQNTPIGDGPVLLPDNHYLSVQSKLSKDPNEKRDHMVLLEFVTAAGITLGMDELYKGGTGGSMVSKGEEL
FTGVVPILVELDGDVNGHKFSVSGEGEGDATYGKLTLKFICTTGKLPVPWPTLVTTLTYGVQCESRYPDH
MKQHDFFKSAMPEGYIQERTIFFKDDGNYKTRAEVKFEGDTLVNRIELKGIDFKEDGNILGHKLEYNASR
GVMTAFMQALGFTAQQGGWEKC
PanZ(G116)-AS-cpGFP-AS-(F117)PanZ Amino Acid Sequence
SEQ ID NO: 11
MKLTIIRLEKESDQDRIDLQKIWPEYSPSSLQVDDNHRIYAARFNERLLAAVRVTLSGTEGALDSLRVRE
VTRRRGVGQYLLEEVLRNNPGVSCWWMADAGVEDRGVMTAFMQALGASNVYIKADKQKNGIKANFKIRHN
IEDGGVQLAYHYQQNTPIGDGPVLLPDNHYLSVQSKLSKDPNEKRDHMVLLEFVTAAGITLGMDELYKGG
TGGSMVSKGEELFTGVVPILVELDGDVNGHKESVSGEGEGDATYGKLTLKFICTTGKLPVPWPTLVTTLT
YGVQCFSRYPDHMKQHDFFKSAMPEGYIQERTIFFKDDGNYKTRAEVKFEGDTLVNRIELKGIDFKEDGN
ILGHKLEYNASFTAQQGGWEKC
PanZ(R69)-GAS-cpGFP-G-(E70)PanZ Amino Acid Sequence
SEQ ID NO: 12
KLTIIRLEKESDQDRIDLQKIWPEYSPSSLQVDDNHRIYAARFNERLLAAVRVTLSGTEGALDSLRVRGA
SNVYIKADKQKNGIKANFKIRHNIEDGGVQLAYHYQQNTPIGDGPVLLPDNHYLSVQSKLSKDPNEKRDH
MVLLEFVTAAGITLGMDELYKGGTGGSMVSKGEELFTGVVPILVELDGDVNGHKESVSGEGEGDATYGKL
TLKFICTTGKLPVPWPTLVTTLTYGVQCFSRYPDHMKQHDFFKSAMPEGYIQERTIFFKDDGNYKTRAEV
KFEGDTLVNRIELKGIDFKEDGNILGHKLEYNGEVTRRRGVGQYLLEEVLRNNPGVSCWWMADAGVEDRG
VMTAFMQALGFTAQQGGWEKC
PanZ(R69)-GAS-cpGFP-(E70)PanZ Amino Acid Sequence
SEQ ID NO: 13
MKLTIIRLEKFSDQDRIDLQKIWPEYSPSSLQVDDNHRIYAARFNERLLAAVRVTLSGTEGALDSLRVRG
ASNVYIKADKQKNGIKANFKIRHNIEDGGVQLAYHYQQNTPIGDGPVLLPDNHYLSVQSKLSKDPNEKRD
HMVLLEFVTAAGITLGMDELYKGGTGGSMVSKGEELFTGVVPILVELDGDVNGHKFSVSGEGEGDATYGK
LTLKFICTTGKLPVPWPTLVTTLTYGVQCFSRYPDHMKQHDFFKSAMPEGYIQERTIFFKDDGNYKTRAE
VKFEGDTLVNRIELKGIDFKEDGNILGHKLEYNEVTRRRGVGQYLLEEVLRNNPGVSCWWMADAGVEDRG
VMTAFMQALGFTAQQGGWEKC
PanZ(R69)-GA-cpGFP-(E70)PanZ Amino Acid Sequence
SEQ ID NO: 14
MKLTIIRLEKESDQDRIDLQKIWPEYSPSSLQVDDNHRIYAARFNERLLAAVRVTLSGTEGALDSLRVRG
ANVYIKADKQKNGIKANFKIRHNIEDGGVQLAYHYQQNTPIGDGPVLLPDNHYLSVQSKLSKDPNEKRDH
MVLLEFVTAAGITLGMDELYKGGTGGSMVSKGEELFTGVVPILVELDGDVNGHKESVSGEGEGDATYGKL
TLKFICTTGKLPVPWPTLVTTLTYGVQCFSRYPDHMKQHDFFKSAMPEGYIQERTIFFKDDGNYKTRAEV
KFEGDTLVNRIELKGIDFKEDGNILGHKLEYNEVTRRRGVGQYLLEEVLRNNPGVSCWWMADAGVEDRGV
MTAFMQALGFTAQQGGWEKC
PanZ(R69)-cpGFP-(E70)PanZ Amino Acid Sequence
SEQ ID NO: 15
MKLTIIRLEKESDQDRIDLQKIWPEYSPSSLQVDDNHRIYAARFNERLLAAVRVTLSGTEGALDSLRVRN
VYIKADKQKNGIKANFKIRHNIEDGGVQLAYHYQQNTPIGDGPVLLPDNHYLSVQSKLSKDPNEKRDHMV
LLEFVTAAGITLGMDELYKGGTGGSMVSKGEELFTGVVPILVELDGDVNGHKESVSGEGEGDATYGKLTL
KFICTTGKLPVPWPTLVTTLTYGVQCFSRYPDHMKQHDFFKSAMPEGYIQERTIFFKDDGNYKTRAEVKF
EGDTLVNRIELKGIDFKEDGNILGHKLEYNEVTRRRGVGQYLLEEVLRNNPGVSCWWMADAGVEDRGVMT
AFMQALGFTAQQGGWEKC
PanZ(69)-G-cpGFP-GAS-(E70)PanZ Amino Acid Sequence
SEQ ID NO: 16
MKLTIIRLEKFSDQDRIDLQKIWPEYSPSSLQVDDNHRIYAARFNERLLAAVRVTLSGTEGALDSLRVRG
NVYIKADKQKNGIKANFKIRHNIEDGGVQLAYHYQQNTPIGDGPVLLPDNHYLSVQSKLSKDPNEKRDHM
VLLEFVTAAGITLGMDELYKGGTGGSMVSKGEELFTGVVPILVELDGDVNGHKFSVSGEGEGDATYGKLT
LKFICTTGKLPVPWPTLVTTLTYGVQCFSRYPDHMKQHDFFKSAMPEGYIQERTIFFKDDGNYKTRAEVK
FEGDTLVNRIELKGIDFKEDGNILGHKLEYNGASEVTRRRGVGQYLLEEVLRNNPGVSCWWMADAGVEDR
GVMTAFMQALGFTAQQGGWEKC
PanZ(R69)-cpGFP-GAS-(E70)PanZ Amino Acid Sequence
SEQ ID NO: 17
MKLTIIRLEKESDQDRIDLQKIWPEYSPSSLQVDDNHRIYAARFNERLLAAVRVTLSGTEGALDSLRVRN
VYIKADKQKNGIKANFKIRHNIEDGGVQLAYHYQQNTPIGDGPVLLPDNHYLSVQSKLSKDPNEKRDHMV
LLEFVTAAGITLGMDELYKGGTGGSMVSKGEELFTGVVPILVELDGDVNGHKESVSGEGEGDATYGKLTL
KFICTTGKLPVPWPTLVTTLTYGVQCFSRYPDHMKQHDFFKSAMPEGYIQERTIFFKDDGNYKTRAEVKF
EGDTLVNRIELKGIDFKEDGNILGHKLEYNGASEVTRRRGVGQYLLEEVLRNNPGVSCWWMADAGVEDRG
VMTAFMQALGFTAQQGGWEKC
PanZ(R69)-GA-cpYFP-GA-(E70)PanZ Amino Acid Sequence
SEQ ID NO: 18
MKLTIIRLEKFSDQDRIDLQKIWPEYSPSSLQVDDNHRIYAARFNERLLAAVRVTLSGTEGALDSLRVRG
ANVYIKADKQKNGIKANFKIRHNIEDGGVQLAYHYQQNTPIGDGPVLLPDNHYLSYQSVLSKDPNEKRDH
MVLLEFVTAAGITLGMDELYKGGTGGSMVSKGEELFTGVVPILVELDGDVNGHKFSVSGEGEGDATYGKL
TLKFICTTGKLPVPWPTLVTTLTYGVQCFSRYPDHMKQHDFFKSAMPEGYIQERTIFFKDDGNYKTRAEV
KFEGDTLVNRIELKGIDFKEDGNILGHKLEYNGAEVTRRRGVGQYLLEEVLRNNPGVSCWWMADAGVEDR
GVMTAFMQALGFTAQQGGWEKC
PanZ(R69)-GA-cpBFP-GA-(E70)PanZ Amino Acid Sequence
SEQ ID NO: 19
MKLTIIRLEKFSDQDRIDLQKIWPEYSPSSLQVDDNHRIYAARFNERLLAAVRVTLSGTEGALDSLRVRG
ANVYIKADKQKNGIKANFKIRHNIEDGGVQLAYHYQQNTPIGDGPVLLPDNHYLSVQSKLSKDPNEKRDH
MVLLEFVTAAGITLGMDELYKGGTGGSMVSKGEELFTGVVPILVELDGDVNGHKESVSGEGEGDATYGKL
TLKFICTTGKLPVPWPTLVTTLSHGVQCFSRYPDHMKQHDFFKSAMPEGYIQERTIFFKDDGNYKTRAEV
KFEGDTLVNRIELKGIDFKEDGNILGHKLEYNGAEVTRRRGVGQYLLEEVLRNNPGVSCWWMADAGVEDR
GVMTAFMQALGFTAQQGGWEKC
PanZ Nucleic Acid Sequence
SEQ ID NO: 20
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAGAG
GTGACAAGACGGAGAGGGGTAGGACAATATTTGCTCGAGGAGGTCCTTAGAAATAACCCCGGAGTAAGTT
GCTGGTGGATGGCGGACGCTGGAGTTGAGGATCGCGGAGTCATGACGGCTTTCATGCAGGCCCTTGGGTT
CACCGCACAGCAAGGGGGCTGGGAGAAGTGC
cpGFP Nucleic Acid Sequence
SEQ ID NO: 21
AACGTCTATATCAAGGCCGACAAGCAGAAGAACGGCATCAAGGCGAACTTCAAGATCCGCCACAACATCG
AGGACGGCGGCGTGCAGCTCGCCTACCACTACCAGCAGAACACCCCCATCGGCGACGGCCCCGTGCTGCT
GCCCGACAACCACTACCTGAGCGTGCAGTCCAAACTTTCGAAAGACCCCAACGAGAAGCGCGATCACATG
GTCCTGCTGGAGTTCGTGACCGCCGCCGGGATCACTCTCGGCATGGACGAGCTGTACAAGGGCGGTACCG
GAGGGAGCATGGTGAGCAAGGGCGAGGAGCTGTTCACCGGGGTGGTGCCCATCCTGGTCGAGCTGGACGG
CGACGTAAACGGCCACAAGTTCAGCGTGTCCGGCGAGGGTGAGGGCGATGCCACCTACGGCAAGCTGACC
CTGAAGTTCATCTGCACCACCGGCAAGCTGCCCGTGCCCTGGCCCACCCTCGTGACCACCCTGACCTACG
GCGTGCAGTGCTTCAGCCGCTACCCCGACCACATGAAGCAGCACGACTTCTTCAAGTCCGCCATGCCCGA
AGGCTACATCCAGGAGCGCACCATCTTCTTCAAGGACGACGGCAACTACAAGACCCGCGCCGAGGTGAAG
TTCGAGGGCGACACCCTGGTGAACCGCATCGAGCTGAAGGGCATCGACTTCAAGGAGGACGGCAACATCC
TGGGGCACAAGCTGGAGTACAAC
cpYFP Nucleic Acid Sequence
SEQ ID NO: 22
AACGTCTATATCAAGGCCGACAAGCAGAAGAACGGCATCAAGGCGAACTTCAAGATCCGCCACAACATCG
AGGACGGCGGCGTGCAGCTCGCCTACCACTACCAGCAGAACACCCCCATCGGCGACGGCCCCGTGCTGCT
GCCCGACAACCACTACCTGAGCTATCAGTCCGTACTTTCGAAAGACCCCAACGAGAAGCGCGATCACATG
GTCCTGCTGGAGTTCGTGACCGCCGCCGGGATCACTCTCGGCATGGACGAGCTGTACAAGGGCGGTACCG
GAGGGAGCATGGTGAGCAAGGGCGAGGAGCTGTTCACCGGGGTGGTGCCCATCCTGGTCGAGCTGGACGG
CGACGTAAACGGCCACAAGTTCAGCGTGTCCGGCGAGGGTGAGGGCGATGCCACCTACGGCAAGCTGACC
CTGAAGTTCATCTGCACCACCGGCAAGCTGCCCGTGCCCTGGCCCACCCTCGTGACCACCCTGACCTACG
GCGTGCAGTGCTTCAGCCGCTACCCCGACCACATGAAGCAGCACGACTTCTTCAAGTCCGCCATGCCCGA
AGGCTACATCCAGGAGCGCACCATCTTCTTCAAGGACGACGGCAACTACAAGACCCGCGCCGAGGTGAAG
TTCGAGGGCGACACCCTGGTGAACCGCATCGAGCTGAAGGGCATCGACTTCAAGGAGGACGGCAACATCC
TGGGGCACAAGCTGGAGTACAAC
cpBFP Nucleic Acid Sequence
SEQ ID NO: 23
AACGTCTATATCAAGGCCGACAAGCAGAAGAACGGCATCAAGGCGAACTTCAAGATCCGCCACAACATCG
AGGACGGCGGCGTGCAGCTCGCCTACCACTACCAGCAGAACACCCCCATCGGCGACGGCCCCGTGCTGCT
GCCCGACAACCACTACCTGAGCGTGCAGTCCAAACTTTCGAAAGACCCCAACGAGAAGCGCGATCACATG
GTCCTGCTGGAGTTCGTGACCGCCGCCGGGATCACTCTCGGCATGGACGAGCTGTACAAGGGCGGTACCG
GAGGGAGCATGGTGAGCAAGGGCGAGGAGCTGTTCACCGGGGTGGTGCCCATCCTGGTCGAGCTGGACGG
CGACGTAAACGGCCACAAGTTCAGCGTGTCCGGCGAGGGTGAGGGCGATGCCACCTACGGCAAGCTGACC
CTGAAGTTCATCTGCACCACCGGCAAGCTGCCCGTGCCCTGGCCCACCCTCGTGACCACCCTGTCCCACG
GCGTGCAGTGCTTCAGCCGCTACCCCGACCACATGAAGCAGCACGACTTCTTCAAGTCCGCCATGCCCGA
AGGCTACATCCAGGAGCGCACCATCTTCTTCAAGGACGACGGCAACTACAAGACCCGCGCCGAGGTGAAG
TTCGAGGGCGACACCCTGGTGAACCGCATCGAGCTGAAGGGCATCGACTTCAAGGAGGACGGCAACATCC
TGGGGCACAAGCTGGAGTACAAC
PanZ(R69)-GA-cpGFP-GA-(E70)PanZ (“PANcACe”) Nucleic Acid Sequence
SEQ ID NO: 24
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAGGA
GCAAACGTCTATATCAAGGCCGACAAGCAGAAGAACGGCATCAAGGCGAACTTCAAGATCCGCCACAACA
TCGAGGACGGCGGCGTGCAGCTCGCCTACCACTACCAGCAGAACACCCCCATCGGCGACGGCCCCGTGCT
GCTGCCCGACAACCACTACCTGAGCGTGCAGTCCAAACTTTCGAAAGACCCCAACGAGAAGCGCGATCAC
ATGGTCCTGCTGGAGTTCGTGACCGCCGCCGGGATCACTCTCGGCATGGACGAGCTGTACAAGGGCGGTA
CCGGAGGGAGCATGGTGAGCAAGGGCGAGGAGCTGTTCACCGGGGTGGTGCCCATCCTGGTCGAGCTGGA
CGGCGACGTAAACGGCCACAAGTTCAGCGTGTCCGGCGAGGGTGAGGGCGATGCCACCTACGGCAAGCTG
ACCCTGAAGTTCATCTGCACCACCGGCAAGCTGCCCGTGCCCTGGCCCACCCTCGTGACCACCCTGACCT
ACGGCGTGCAGTGCTTCAGCCGCTACCCCGACCACATGAAGCAGCACGACTTCTTCAAGTCCGCCATGCC
CGAAGGCTACATCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACGGCAACA
TCCTGGGGCACAAGCTGGAGTACAACGGTGCTGAGGTGACAAGACGGAGAGGGGTAGGACAATATTTGCT
CGAGGAGGTCCTTAGAAATAACCCCGGAGTAAGTTGCTGGTGGATGGCGGACGCTGGAGTTGAGGATCGC
GGAGTCATGACGGCTTTCATGCAGGCCCTTGGGTTCACCGCACAGCAAGGGGGCTGGGAGAAGTGC
PanZ(W23)-AS-cpGFP-AS-(P24)PanZ Nucleic Acid Sequence
SEQ ID NO: 25
ATGAAGCTGACAATCATTCGCCTGGAGAAATTTTCCGACCAGGATAGAATCGACCTTCAGAAGATCTGGG
CATCTAACGTCTATATCAAGGCCGACAAGCAGAAGAACGGCATCAAGGCGAACTTCAAGATCCGCCACAA
CATCGAGGACGGCGGCGTGCAGCTCGCCTACCACTACCAGCAGAACACCCCCATCGGCGACGGCCCCGTG
CTGCTGCCCGACAACCACTACCTGAGCGTGCAGTCCAAACTTTCGAAAGACCCCAACGAGAAGCGCGATC
ACATGGTCCTGCTGGAGTTCGTGACCGCCGCCGGGATCACTCTCGGCATGGACGAGCTGTACAAGGGCGG
TACCGGAGGGAGCATGGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGTGAGGGCGATGCCACCTACGGCAAGC
TGACCCTGAAGTTCATCTGCACCACCGGCAAGCTGCCCGTGCCCTGGCCCACCCTCGTGACCACCCTGAC
CTACGGCGTGCAGTGCTTCAGCCGCTACCCCGACCACATGAAGCAGCACGACTTCTTCAAGTCCGCCATG
CCCGAAGGCTACATCCAGGAGCGCACCATCTTCTTCAAGGACGACGGCAACTACAAGACCCGCGCCGAGG
TGAAGTTCGAGGGCGACACCCTGGTGAACCGCATCGAGCTGAAGGGCATCGACTTCAAGGAGGACGGCAA
CATCCTGGGGCACAAGCTGGAGTACAACGCGTCACCCGAATATTCTCCCAGTAGCCTCCAAGTAGACGAC
AACCATCGAATCTACGCCGCGAGATTTAATGAACGACTGCTTGCGGCGGTTAGGGTAACCCTCAGCGGTA
CGGAAGGTGCTCTGGACTCCCTGCGGGTCCGAGAGGTGACAAGACGGAGAGGGGTAGGACAATATTTGCT
CGAGGAGGTCCTTAGAAATAACCCCGGAGTAAGTTGCTGGTGGATGGCGGACGCTGGAGTTGAGGATCGC
GGAGTCATGACGGCTTTCATGCAGGCCCTTGGGTTCACCGCACAGCAAGGGGGCTGGGAGAAGTGC
PanZ(V71)-AS-cpGFP-AS-(T72)PanZ Nucleic Acid Sequence
SEQ ID NO: 26
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAGAG
GTGGCATCTAACGTCTATATCAAGGCCGACAAGCAGAAGAACGGCATCAAGGCGAACTTCAAGATCCGCC
ACAACATCGAGGACGGCGGCGTGCAGCTCGCCTACCACTACCAGCAGAACACCCCCATCGGCGACGGCCC
CGTGCTGCTGCCCGACAACCACTACCTGAGCGTGCAGTCCAAACTTTCGAAAGACCCCAACGAGAAGCGC
GATCACATGGTCCTGCTGGAGTTCGTGACCGCCGCCGGGATCACTCTCGGCATGGACGAGCTGTACAAGG
GCGGTACCGGAGGGAGCATGGTGAGCAAGGGCGAGGAGCTGTTCACCGGGGTGGTGCCCATCCTGGTCGA
GCTGGACGGCGACGTAAACGGCCACAAGTTCAGCGTGTCCGGCGAGGGTGAGGGCGATGCCACCTACGGC
AAGCTGACCCTGAAGTTCATCTGCACCACCGGCAAGCTGCCCGTGCCCTGGCCCACCCTCGTGACCACCC
TGACCTACGGCGTGCAGTGCTTCAGCCGCTACCCCGACCACATGAAGCAGCACGACTTCTTCAAGTCCGC
CATGCCCGAAGGCTACATCCAGGAGCGCACCATCTTCTTCAAGGACGACGGCAACTACAAGACCCGCGCC
GAGGTGAAGTTCGAGGGCGACACCCTGGTGAACCGCATCGAGCTGAAGGGCATCGACTTCAAGGAGGACG
GCAACATCCTGGGGCACAAGCTGGAGTACAACGCGTCAACAAGACGGAGAGGGGTAGGACAATATTTGCT
CGAGGAGGTCCTTAGAAATAACCCCGGAGTAAGTTGCTGGTGGATGGCGGACGCTGGAGTTGAGGATCGC
GGAGTCATGACGGCTTTCATGCAGGCCCTTGGGTTCACCGCACAGCAAGGGGGCTGGGAGAAGTGC
PanZ(R69)-AS-cpGFP-AS-(E70)PanZ Nucleic Acid Sequence
SEQ ID NO: 27
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAGCA
TCTAACGTCTATATCAAGGCCGACAAGCAGAAGAACGGCATCAAGGCGAACTTCAAGATCCGCCACAACA
TCGAGGACGGCGGCGTGCAGCTCGCCTACCACTACCAGCAGAACACCCCCATCGGCGACGGCCCCGTGCT
GCTGCCCGACAACCACTACCTGAGCGTGCAGTCCAAACTTTCGAAAGACCCCAACGAGAAGCGCGATCAC
ATGGTCCTGCTGGAGTTCGTGACCGCCGCCGGGATCACTCTCGGCATGGACGAGCTGTACAAGGGCGGTA
CCGGAGGGAGCATGGTGAGCAAGGGCGAGGAGCTGTTCACCGGGGTGGTGCCCATCCTGGTCGAGCTGGA
CGGCGACGTAAACGGCCACAAGTTCAGCGTGTCCGGCGAGGGTGAGGGCGATGCCACCTACGGCAAGCTG
ACCCTGAAGTTCATCTGCACCACCGGCAAGCTGCCCGTGCCCTGGCCCACCCTCGTGACCACCCTGACCT
ACGGCGTGCAGTGCTTCAGCCGCTACCCCGACCACATGAAGCAGCACGACTTCTTCAAGTCCGCCATGCC
CGAAGGCTACATCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACGGCAACA
TCCTGGGGCACAAGCTGGAGTACAACGCGTCAGAGGTGACAAGACGGAGAGGGGTAGGACAATATTTGCT
CGAGGAGGTCCTTAGAAATAACCCCGGAGTAAGTTGCTGGTGGATGGCGGACGCTGGAGTTGAGGATCGC
GGAGTCATGACGGCTTTCATGCAGGCCCTTGGGTTCACCGCACAGCAAGGGGGCTGGGAGAAGTGC
PanZ(D99)-AS-cpGFP-AS-(A100)PanZ Nucleic Acid Sequence
SEQ ID NO: 28
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAGAG
GTGACAAGACGGAGAGGGGTAGGACAATATTTGCTCGAGGAGGTCCTTAGAAATAACCCCGGAGTAAGTT
GCTGGTGGATGGCGGACGCATCTAACGTCTATATCAAGGCCGACAAGCAGAAGAACGGCATCAAGGCGAA
CTTCAAGATCCGCCACAACATCGAGGACGGCGGCGTGCAGCTCGCCTACCACTACCAGCAGAACACCCCC
ATCGGCGACGGCCCCGTGCTGCTGCCCGACAACCACTACCTGAGCGTGCAGTCCAAACTTTCGAAAGACC
CCAACGAGAAGCGCGATCACATGGTCCTGCTGGAGTTCGTGACCGCCGCCGGGATCACTCTCGGCATGGA
CGAGCTGTACAAGGGCGGTACCGGAGGGAGCATGGTGAGCAAGGGCGAGGAGCTGTTCACCGGGGTGGTG
CCCATCCTGGTCGAGCTGGACGGCGACGTAAACGGCCACAAGTTCAGCGTGTCCGGCGAGGGTGAGGGCG
ATGCCACCTACGGCAAGCTGACCCTGAAGTTCATCTGCACCACCGGCAAGCTGCCCGTGCCCTGGCCCAC
CCTCGTGACCACCCTGACCTACGGCGTGCAGTGCTTCAGCCGCTACCCCGACCACATGAAGCAGCACGAC
TTCTTCAAGTCCGCCATGCCCGAAGGCTACATCCAGGAGCGCACCATCTTCTTCAAGGACGACGGCAACT
ACAAGACCCGCGCCGAGGTGAAGTTCGAGGGCGACACCCTGGTGAACCGCATCGAGCTGAAGGGCATCGA
CTTCAAGGAGGACGGCAACATCCTGGGGCACAAGCTGGAGTACAACGCGTCAGCTGGAGTTGAGGATCGC
GGAGTCATGACGGCTTTCATGCAGGCCCTTGGGTTCACCGCACAGCAAGGGGGCTGGGAGAAGTGC
PanZ(D104)-AS-cpGFP-AS-(R105)PanZ Nucleic Acid Sequence
SEQ ID NO: 29
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAGAG
GTGACAAGACGGAGAGGGGTAGGACAATATTTGCTCGAGGAGGTCCTTAGAAATAACCCCGGAGTAAGTT
GCTGGTGGATGGCGGACGCTGGAGTTGAGGATGCATCTAACGTCTATATCAAGGCCGACAAGCAGAAGAA
CGGCATCAAGGCGAACTTCAAGATCCGCCACAACATCGAGGACGGCGGCGTGCAGCTCGCCTACCACTAC
CAGCAGAACACCCCCATCGGCGACGGCCCCGTGCTGCTGCCCGACAACCACTACCTGAGCGTGCAGTCCA
AACTTTCGAAAGACCCCAACGAGAAGCGCGATCACATGGTCCTGCTGGAGTTCGTGACCGCCGCCGGGAT
CACTCTCGGCATGGACGAGCTGTACAAGGGCGGTACCGGAGGGAGCATGGTGAGCAAGGGCGAGGAGCTG
TTCACCGGGGTGGTGCCCATCCTGGTCGAGCTGGACGGCGACGTAAACGGCCACAAGTTCAGCGTGTCCG
GCGAGGGTGAGGGCGATGCCACCTACGGCAAGCTGACCCTGAAGTTCATCTGCACCACCGGCAAGCTGCC
CGTGCCCTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACATCCAGGAGCGCACCATCTTCTTCA
AGGACGACGGCAACTACAAGACCCGCGCCGAGGTGAAGTTCGAGGGCGACACCCTGGTGAACCGCATCGA
GCTGAAGGGCATCGACTTCAAGGAGGACGGCAACATCCTGGGGCACAAGCTGGAGTACAACGCGTCACGC
GGAGTCATGACGGCTTTCATGCAGGCCCTTGGGTTCACCGCACAGCAAGGGGGCTGGGAGAAGTGC
PanZ(G116)-AS-cpGFP-AS-(F117)PanZ Nucleic Acid Sequence
SEQ ID NO: 30
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAGAG
GTGACAAGACGGAGAGGGGTAGGACAATATTTGCTCGAGGAGGTCCTTAGAAATAACCCCGGAGTAAGTT
GCTGGTGGATGGCGGACGCTGGAGTTGAGGATCGCGGAGTCATGACGGCTTTCATGCAGGCCCTTGGGGC
ATCTAACGTCTATATCAAGGCCGACAAGCAGAAGAACGGCATCAAGGCGAACTTCAAGATCCGCCACAAC
ATCGAGGACGGCGGCGTGCAGCTCGCCTACCACTACCAGCAGAACACCCCCATCGGCGACGGCCCCGTGC
TGCTGCCCGACAACCACTACCTGAGCGTGCAGTCCAAACTTTCGAAAGACCCCAACGAGAAGCGCGATCA
CATGGTCCTGCTGGAGTTCGTGACCGCCGCCGGGATCACTCTCGGCATGGACGAGCTGTACAAGGGCGGT
ACCGGAGGGAGCATGGTGAGCAAGGGCGAGGAGCTGTTCACCGGGGTGGTGCCCATCCTGGTCGAGCTGG
ACGGCGACGTAAACGGCCACAAGTTCAGCGTGTCCGGCGAGGGTGAGGGCGATGCCACCTACGGCAAGCT
GACCCTGAAGTTCATCTGCACCACCGGCAAGCTGCCCGTGCCCTGGCCCACCCTCGTGACCACCCTGACC
TACGGCGTGCAGTGCTTCAGCCGCTACCCCGACCACATGAAGCAGCACGACTTCTTCAAGTCCGCCATGC
CCGAAGGCTACATCCAGGAGCGCACCATCTTCTTCAAGGACGACGGCAACTACAAGACCCGCGCCGAGGT
GAAGTTCGAGGGCGACACCCTGGTGAACCGCATCGAGCTGAAGGGCATCGACTTCAAGGAGGACGGCAAC
ATCCTGGGGCACAAGCTGGAGTACAACGCGTCATTCACCGCACAGCAAGGGGGCTGGGAGAAGTGC
PanZ(R69)-GAS-cpGFP-G-(E70)PanZ Nucleic Acid Sequence
SEQ ID NO: 31
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAGGA
GCAAGTAACGTCTATATCAAGGCCGACAAGCAGAAGAACGGCATCAAGGCGAACTTCAAGATCCGCCACA
ACATCGAGGACGGCGGCGTGCAGCTCGCCTACCACTACCAGCAGAACACCCCCATCGGCGACGGCCCCGT
GCTGCTGCCCGACAACCACTACCTGAGCGTGCAGTCCAAACTTTCGAAAGACCCCAACGAGAAGCGCGAT
CACATGGTCCTGCTGGAGTTCGTGACCGCCGCCGGGATCACTCTCGGCATGGACGAGCTGTACAAGGGCG
GTACCGGAGGGAGCATGGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGTGAGGGCGATGCCACCTACGGCAAG
CTGACCCTGAAGTTCATCTGCACCACCGGCAAGCTGCCCGTGCCCTGGCCCACCCTCGTGACCACCCTGA
CCTACGGCGTGCAGTGCTTCAGCCGCTACCCCGACCACATGAAGCAGCACGACTTCTTCAAGTCCGCCAT
GCCCGAAGGCTACATCCAGGAGCGCACCATCTTCTTCAAGGACGACGGCAACTACAAGACCCGCGCCGAG
GTGAAGTTCGAGGGCGACACCCTGGTGAACCGCATCGAGCTGAAGGGCATCGACTTCAAGGAGGACGGCA
ACATCCTGGGGCACAAGCTGGAGTACAACGGTGAGGTGACAAGACGGAGAGGGGTAGGACAATATTTGCT
CGAGGAGGTCCTTAGAAATAACCCCGGAGTAAGTTGCTGGTGGATGGCGGACGCTGGAGTTGAGGATCGC
GGAGTCATGACGGCTTTCATGCAGGCCCTTGGGTTCACCGCACAGCAAGGGGGCTGGGAGAAGTGC
PanZ(R69)-GAS-cpGFP-(E70)PanZ Nucleic Acid Sequence
SEQ ID NO: 32
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAGGA
GCAAGTAACGTCTATATCAAGGCCGACAAGCAGAAGAACGGCATCAAGGCGAACTTCAAGATCCGCCACA
ACATCGAGGACGGCGGCGTGCAGCTCGCCTACCACTACCAGCAGAACACCCCCATCGGCGACGGCCCCGT
GCTGCTGCCCGACAACCACTACCTGAGCGTGCAGTCCAAACTTTCGAAAGACCCCAACGAGAAGCGCGAT
CACATGGTCCTGCTGGAGTTCGTGACCGCCGCCGGGATCACTCTCGGCATGGACGAGCTGTACAAGGGCG
GTACCGGAGGGAGCATGGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGTGAGGGCGATGCCACCTACGGCAAG
CTGACCCTGAAGTTCATCTGCACCACCGGCAAGCTGCCCGTGCCCTGGCCCACCCTCGTGACCACCCTGA
CCTACGGCGTGCAGTGCTTCAGCCGCTACCCCGACCACATGAAGCAGCACGACTTCTTCAAGTCCGCCAT
GCCCGAAGGCTACATCCAGGAGCGCACCATCTTCTTCAAGGACGACGGCAACTACAAGACCCGCGCCGAG
GTGAAGTTCGAGGGCGACACCCTGGTGAACCGCATCGAGCTGAAGGGCATCGACTTCAAGGAGGACGGCA
ACATCCTGGGGCACAAGCTGGAGTACAACGAGGTGACAAGACGGAGAGGGGTAGGACAATATTTGCTCGA
GGAGGTCCTTAGAAATAACCCCGGAGTAAGTTGCTGGTGGATGGCGGACGCTGGAGTTGAGGATCGCGGA
GTCATGACGGCTTTCATGCAGGCCCTTGGGTTCACCGCACAGCAAGGGGGCTGGGAGAAGTGC
PanZ(R69)-GA-cpGFP-(E70)PanZ Nucleic Acid Sequence
SEQ ID NO: 33
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAGGA
GCAAACGTCTATATCAAGGCCGACAAGCAGAAGAACGGCATCAAGGCGAACTTCAAGATCCGCCACAACA
TCGAGGACGGCGGCGTGCAGCTCGCCTACCACTACCAGCAGAACACCCCCATCGGCGACGGCCCCGTGCT
GCTGCCCGACAACCACTACCTGAGCGTGCAGTCCAAACTTTCGAAAGACCCCAACGAGAAGCGCGATCAC
ATGGTCCTGCTGGAGTTCGTGACCGCCGCCGGGATCACTCTCGGCATGGACGAGCTGTACAAGGGCGGTA
CCGGAGGGAGCATGGTGAGCAAGGGCGAGGAGCTGTTCACCGGGGTGGTGCCCATCCTGGTCGAGCTGGA
CGGCGACGTAAACGGCCACAAGTTCAGCGTGTCCGGCGAGGGTGAGGGCGATGCCACCTACGGCAAGCTG
ACCCTGAAGTTCATCTGCACCACCGGCAAGCTGCCCGTGCCCTGGCCCACCCTCGTGACCACCCTGACCT
ACGGCGTGCAGTGCTTCAGCCGCTACCCCGACCACATGAAGCAGCACGACTTCTTCAAGTCCGCCATGCC
CGAAGGCTACATCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACGGCAACA
TCCTGGGGCACAAGCTGGAGTACAACGAGGTGACAAGACGGAGAGGGGTAGGACAATATTTGCTCGAGGA
GGTCCTTAGAAATAACCCCGGAGTAAGTTGCTGGTGGATGGCGGACGCTGGAGTTGAGGATCGCGGAGTC
ATGACGGCTTTCATGCAGGCCCTTGGGTTCACCGCACAGCAAGGGGGCTGGGAGAAGTGC
PanZ(R69)-cpGFP-(E70)PanZ Nucleic Acid Sequence
SEQ ID NO: 34
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAAAC
GTCTATATCAAGGCCGACAAGCAGAAGAACGGCATCAAGGCGAACTTCAAGATCCGCCACAACATCGAGG
ACGGCGGCGTGCAGCTCGCCTACCACTACCAGCAGAACACCCCCATCGGCGACGGCCCCGTGCTGCTGCC
CGACAACCACTACCTGAGCGTGCAGTCCAAACTTTCGAAAGACCCCAACGAGAAGCGCGATCACATGGTC
CTGCTGGAGTTCGTGACCGCCGCCGGGATCACTCTCGGCATGGACGAGCTGTACAAGGGCGGTACCGGAG
GGAGCATGGTGAGCAAGGGCGAGGAGCTGTTCACCGGGGTGGTGCCCATCCTGGTCGAGCTGGACGGCGA
CGTAAACGGCCACAAGTTCAGCGTGTCCGGCGAGGGTGAGGGCGATGCCACCTACGGCAAGCTGACCCTG
AAGTTCATCTGCACCACCGGCAAGCTGCCCGTGCCCTGGCCCACCCTCGTGACCACCCTGACCTACGGCG
TGCAGTGCTTCAGCCGCTACCCCGACCACATGAAGCAGCACGACTTCTTCAAGTCCGCCATGCCCGAAGG
CTACATCCAGGAGCGCACCATCTTCTTCAAGGACGACGGCAACTACAAGACCCGCGCCGAGGTGAAGTTC
GAGGGCGACACCCTGGTGAACCGCATCGAGCTGAAGGGCATCGACTTCAAGGAGGACGGCAACATCCTGG
GGCACAAGCTGGAGTACAACGAGGTGACAAGACGGAGAGGGGTAGGACAATATTTGCTCGAGGAGGTCCT
TAGAAATAACCCCGGAGTAAGTTGCTGGTGGATGGCGGACGCTGGAGTTGAGGATCGCGGAGTCATGACG
GCTTTCATGCAGGCCCTTGGGTTCACCGCACAGCAAGGGGGCTGGGAGAAGTGC
PanZ(69)-G-cpGFP-GAS-(E70)PanZ Nucleic Acid Sequence
SEQ ID NO: 35
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAGGA
AACGTCTATATCAAGGCCGACAAGCAGAAGAACGGCATCAAGGCGAACTTCAAGATCCGCCACAACATCG
AGGACGGCGGCGTGCAGCTCGCCTACCACTACCAGCAGAACACCCCCATCGGCGACGGCCCCGTGCTGCT
GCCCGACAACCACTACCTGAGCGTGCAGTCCAAACTTTCGAAAGACCCCAACGAGAAGCGCGATCACATG
GTCCTGCTGGAGTTCGTGACCGCCGCCGGGATCACTCTCGGCATGGACGAGCTGTACAAGGGCGGTACCG
GAGGGAGCATGGTGAGCAAGGGCGAGGAGCTGTTCACCGGGGTGGTGCCCATCCTGGTCGAGCTGGACGG
CGACGTAAACGGCCACAAGTTCAGCGTGTCCGGCGAGGGTGAGGGCGATGCCACCTACGGCAAGCTGACC
CTGAAGTTCATCTGCACCACCGGCAAGCTGCCCGTGCCCTGGCCCACCCTCGTGACCACCCTGACCTACG
GCGTGCAGTGCTTCAGCCGCTACCCCGACCACATGAAGCAGCACGACTTCTTCAAGTCCGCCATGCCCGA
AGGCTACATCCAGGAGCGCACCATCTTCTTCAAGGACGACGGCAACTACAAGACCCGCGCCGAGGTGAAG
TTCGAGGGCGACACCCTGGTGAACCGCATCGAGCTGAAGGGCATCGACTTCAAGGAGGACGGCAACATCC
TGGGGCACAAGCTGGAGTACAACGGTGCTTCAGAGGTGACAAGACGGAGAGGGGTAGGACAATATTTGCT
CGAGGAGGTCCTTAGAAATAACCCCGGAGTAAGTTGCTGGTGGATGGCGGACGCTGGAGTTGAGGATCGC
GGAGTCATGACGGCTTTCATGCAGGCCCTTGGGTTCACCGCACAGCAAGGGGGCTGGGAGAAGTGC
PanZ(R69)-cpGFP-GAS-(E70)PanZ Nucleic Acid Sequence
SEQ ID NO: 36
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAAAC
GTCTATATCAAGGCCGACAAGCAGAAGAACGGCATCAAGGCGAACTTCAAGATCCGCCACAACATCGAGG
ACGGCGGCGTGCAGCTCGCCTACCACTACCAGCAGAACACCCCCATCGGCGACGGCCCCGTGCTGCTGCC
CGACAACCACTACCTGAGCGTGCAGTCCAAACTTTCGAAAGACCCCAACGAGAAGCGCGATCACATGGTC
CTGCTGGAGTTCGTGACCGCCGCCGGGATCACTCTCGGCATGGACGAGCTGTACAAGGGCGGTACCGGAG
GGAGCATGGTGAGCAAGGGCGAGGAGCTGTTCACCGGGGTGGTGCCCATCCTGGTCGAGCTGGACGGCGA
CGTAAACGGCCACAAGTTCAGCGTGTCCGGCGAGGGTGAGGGCGATGCCACCTACGGCAAGCTGACCCTG
AAGTTCATCTGCACCACCGGCAAGCTGCCCGTGCCCTGGCCCACCCTCGTGACCACCCTGACCTACGGCG
TGCAGTGCTTCAGCCGCTACCCCGACCACATGAAGCAGCACGACTTCTTCAAGTCCGCCATGCCCGAAGG
CTACATCCAGGAGCGCACCATCTTCTTCAAGGACGACGGCAACTACAAGACCCGCGCCGAGGTGAAGTTC
GAGGGCGACACCCTGGTGAACCGCATCGAGCTGAAGGGCATCGACTTCAAGGAGGACGGCAACATCCTGG
GGCACAAGCTGGAGTACAACGGTGCTTCAGAGGTGACAAGACGGAGAGGGGTAGGACAATATTTGCTCGA
GGAGGTCCTTAGAAATAACCCCGGAGTAAGTTGCTGGTGGATGGCGGACGCTGGAGTTGAGGATCGCGGA
GTCATGACGGCTTTCATGCAGGCCCTTGGGTTCACCGCACAGCAAGGGGGCTGGGAGAAGTGC
PanZ(R69)-GA-cpYFP-GA-(E70)PanZ Nucleic Acid Sequence
SEQ ID NO: 37
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAGGA
GCAAACGTCTATATCAAGGCCGACAAGCAGAAGAACGGCATCAAGGCGAACTTCAAGATCCGCCACAACA
TCGAGGACGGCGGCGTGCAGCTCGCCTACCACTACCAGCAGAACACCCCCATCGGCGACGGCCCCGTGCT
GCTGCCCGACAACCACTACCTGAGCTATCAGTCCGTACTTTCGAAAGACCCCAACGAGAAGCGCGATCAC
ATGGTCCTGCTGGAGTTCGTGACCGCCGCCGGGATCACTCTCGGCATGGACGAGCTGTACAAGGGCGGTA
CCGGAGGGAGCATGGTGAGCAAGGGCGAGGAGCTGTTCACCGGGGTGGTGCCCATCCTGGTCGAGCTGGA
CGGCGACGTAAACGGCCACAAGTTCAGCGTGTCCGGCGAGGGTGAGGGCGATGCCACCTACGGCAAGCTG
ACCCTGAAGTTCATCTGCACCACCGGCAAGCTGCCCGTGCCCTGGCCCACCCTCGTGACCACCCTGACCT
ACGGCGTGCAGTGCTTCAGCCGCTACCCCGACCACATGAAGCAGCACGACTTCTTCAAGTCCGCCATGCC
CGAAGGCTACATCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACGGCAACA
TCCTGGGGCACAAGCTGGAGTACAACGGTGCTGAGGTGACAAGACGGAGAGGGGTAGGACAATATTTGCT
CGAGGAGGTCCTTAGAAATAACCCCGGAGTAAGTTGCTGGTGGATGGCGGACGCTGGAGTTGAGGATCGC
GGAGTCATGACGGCTTTCATGCAGGCCCTTGGGTTCACCGCACAGCAAGGGGGCTGGGAGAAGTGC
PanZ(R69)-GA-cpBFP-GA-(E70)PanZ Nucleic Acid Sequence
SEQ ID NO: 38
ATGAAGCTGACAATCATTCGCCTGGAGAAATTTTCCGACCAGGATAGAATCGACCTTCAGAAGATCTGGC
CCGAATATTCTCCCAGTAGCCTCCAAGTAGACGACAACCATCGAATCTACGCCGCGAGATTTAATGAACG
ACTGCTTGCGGCGGTTAGGGTAACCCTCAGCGGTACGGAAGGTGCTCTGGACTCCCTGCGGGTCCGAGGA
GCAAACGTCTATATCAAGGCCGACAAGCAGAAGAACGGCATCAAGGCGAACTTCAAGATCCGCCACAACA
TCGAGGACGGCGGCGTGCAGCTCGCCTACCACTACCAGCAGAACACCCCCATCGGCGACGGCCCCGTGCT
GCTGCCCGACAACCACTACCTGAGCGTGCAGTCCAAACTTTCGAAAGACCCCAACGAGAAGCGCGATCAC
ATGGTCCTGCTGGAGTTCGTGACCGCCGCCGGGATCACTCTCGGCATGGACGAGCTGTACAAGGGCGGTA
CCGGAGGGAGCATGGTGAGCAAGGGCGAGGAGCTGTTCACCGGGGTGGTGCCCATCCTGGTCGAGCTGGA
CGGCGACGTAAACGGCCACAAGTTCAGCGTGTCCGGCGAGGGTGAGGGCGATGCCACCTACGGCAAGCTG
ACCCTGAAGTTCATCTGCACCACCGGCAAGCTGCCCGTGCCCTGGCCCACCCTCGTGACCACCCTGTCCC
ACGGCGTGCAGTGCTTCAGCCGCTACCCCGACCACATGAAGCAGCACGACTTCTTCAAGTCCGCCATGCC
CGAAGGCTACATCCAGGAGCGCACCATCTTCTTCAAGGACGACGGCAACTACAAGACCCGCGCCGAGGTG
AAGTTCGAGGGCGACACCCTGGTGAACCGCATCGAGCTGAAGGGCATCGACTTCAAGGAGGACGGCAACA
TCCTGGGGCACAAGCTGGAGTACAACGGTGCTGAGGTGACAAGACGGAGAGGGGTAGGACAATATTTGCT
CGAGGAGGTCCTTAGAAATAACCCCGGAGTAAGTTGCTGGTGGATGGCGGACGCTGGAGTTGAGGATCGC
GGAGTCATGACGGCTTTCATGCAGGCCCTTGGGTTCACCGCACAGCAAGGGGGCTGGGAGAAGTGC
6xHistidine-TEV-PanZ Amino Acid Sequence
SEQ ID NO: 39
MHHHHHHENLYFQSMKLTIIRLEKESDQDRIDLQKIWPEYSPSSLQVDDNHRIYAARFNERLLAAVRVTL
SGTEGALDSLRVREVTRRRGVGQYLLEEVLRNNPGVSCWWMADAGVEDRGVMTAFMQALGFTAQQGGWEK
C
6xHistidine-TEV-cpGFP Amino Acid Sequence
SEQ ID NO: 40
MHHHHHHENLYFQSNVYIKADKQKNGIKANFKIRHNIEDGGVQLAYHYQQNTPIGDGPVLLPDNHYLSVQ
SKLSKDPNEKRDHMVLLEFVTAAGITLGMDELYKGGTGGSMVSKGEELFTGVVPILVELDGDVNGHKFSV
SGEGEGDATYGKLTLKFICTTGKLPVPWPTLVTTLTYGVQCFSRYPDHMKQHDFFKSAMPEGYIQERTIF
FKDDGNYKTRAEVKFEGDTLVNRIELKGIDFKEDGNILGHKLEYN
6xHistidine-TEV-cpYFP Amino Acid Sequence
SEQ ID NO: 41
MHHHHHHENLYFQSNVYIKADKQKNGIKANFKIRHNIEDGGVQLAYHYQQNTPIGDGPVLLPDNHYLSYQ
SVLSKDPNEKRDHMVLLEFVTAAGITLGMDELYKGGTGGSMVSKGEELFTGVVPILVELDGDVNGHKESV
SGEGEGDATYGKLTLKFICTTGKLPVPWPTLVTTLTYGVQCFSRYPDHMKQHDFFKSAMPEGYIQERTIF
FKDDGNYKTRAEVKFEGDTLVNRIELKGIDFKEDGNILGHKLEYN
6xHistidine-TEV-cpBFP Amino Acid Sequence
SEQ ID NO: 42
MHHHHHHENLYFQSNVYIKADKQKNGIKANFKIRHNIEDGGVQLAYHYQQNTPIGDGPVLLPDNHYLSVQ
SKLSKDPNEKRDHMVLLEFVTAAGITLGMDELYKGGTGGSMVSKGEELFTGVVPILVELDGDVNGHKESV
SGEGEGDATYGKLTLKFICTTGKLPVPWPTLVTTLSHGVQCFSRYPDHMKQHDFFKSAMPEGYIQERTIF
FKDDGNYKTRAEVKFEGDTLVNRIELKGIDFKEDGNILGHKLEYN
6xHistidine-TEV-PanZ(R69)-GA-cpGFP-GA-(E70)PanZ Amino Acid Sequence
SEQ ID NO: 43
MHHHHHHENLYFQSMKLTIIRLEKFSDQDRIDLQKIWPEYSPSSLQVDDNHRIYAARFNERLLAAVRVTL
SGTEGALDSLRVRGANVYIKADKQKNGIKANFKIRHNIEDGGVQLAYHYQQNTPIGDGPVLLPDNHYLSV
QSKLSKDPNEKRDHMVLLEFVTAAGITLGMDELYKGGTGGSMVSKGEELFTGVVPILVELDGDVNGHKES
VSGEGEGDATYGKLTLKFICTTGKLPVPWPTLVTTLTYGVQCFSRYPDHMKQHDFFKSAMPEGYIQERTI
FFKDDGNYKTRAEVKFEGDTLVNRIELKGIDFKEDGNILGHKLEYNGAEVTRRRGVGQYLLEEVLRNNPG
VSCWWMADAGVEDRGVMTAFMQALGFTAQQGGWEKC
6xHistidine-TEV-PanZ(W23)-AS-cpGFP-AS-(P24)PanZ Amino Acid Sequence
SEQ ID NO: 44
MHHHHHHENLYFQSMKLTIIRLEKESDQDRIDLQKIWASNVYIKADKQKNGIKANFKIRHNIEDGGVQLA
YHYQQNTPIGDGPVLLPDNHYLSVQSKLSKDPNEKRDHMVLLEFVTAAGITLGMDELYKGGTGGSMVSKG
EELFTGVVPILVELDGDVNGHKESVSGEGEGDATYGKLTLKFICTTGKLPVPWPTLVTTLTYGVQCFSRY
PDHMKQHDFFKSAMPEGYIQERTIFFKDDGNYKTRAEVKFEGDTLVNRIELKGIDFKEDGNILGHKLEYN
ASPEYSPSSLQVDDNHRIYAARFNERLLAAVRVTLSGTEGALDSLRVREVTRRRGVGQYLLEEVLRNNPG
VSCWWMADAGVEDRGVMTAFMQALGFTAQQGGWEKC
6xHistidine-TEV-PanZ(V71)-AS-cpGFP-AS-(T72)PanZ Amino Acid Sequence
SEQ ID NO: 45
MHHHHHHENLYFQSMKLTIIRLEKFSDQDRIDLQKIWPEYSPSSLQVDDNHRIYAARFNERLLAAVRVTL
SGTEGALDSLRVREVASNVYIKADKQKNGIKANFKIRHNIEDGGVQLAYHYQQNTPIGDGPVLLPDNHYL
SVQSKLSKDPNEKRDHMVLLEFVTAAGITLGMDELYKGGTGGSMVSKGEELFTGVVPILVELDGDVNGHK
FSVSGEGEGDATYGKLTLKFICTTGKLPVPWPTLVTTLTYGVQCFSRYPDHMKQHDFFKSAMPEGYIQER
TIFFKDDGNYKTRAEVKFEGDTLVNRIELKGIDFKEDGNILGHKLEYNASTRRRGVGQYLLEEVLRNNPG
VSCWWMADAGVEDRGVMTAFMQALGFTAQQGGWEKC
6xHistidine-TEV-PanZ(R69)-AS-cpGFP-AS-(E70)PanZ Amino Acid Sequence
SEQ ID NO: 46
MHHHHHHENLYFQSMKLTIIRLEKESDQDRIDLQKIWPEYSPSSLQVDDNHRIYAARFNERLLAAVRVTL
SGTEGALDSLRVRASNVYIKADKQKNGIKANFKIRHNIEDGGVQLAYHYQQNTPIGDGPVLLPDNHYLSV
QSKLSKDPNEKRDHMVLLEFVTAAGITLGMDELYKGGTGGSMVSKGEELFTGVVPILVELDGDVNGHKES
VSGEGEGDATYGKLTLKFICTTGKLPVPWPTLVTTLTYGVQCFSRYPDHMKQHDFFKSAMPEGYIQERTI
FFKDDGNYKTRAEVKFEGDTLVNRIELKGIDFKEDGNILGHKLEYNASEVTRRRGVGQYLLEEVLRNNPG
VSCWWMADAGVEDRGVMTAFMQALGFTAQQGGWEKC
6xHistidine-TEV-PanZ(D99)-AS-cpGFP-AS-(A100)PanZ Amino Acid Sequence
SEQ ID NO: 47
MHHHHHHENLYFQSMKLTIIRLEKESDQDRIDLQKIWPEYSPSSLQVDDNHRIYAARFNERLLAAVRVTL
SGTEGALDSLRVREVTRRRGVGQYLLEEVLRNNPGVSCWWMADASNVYIKADKQKNGIKANFKIRHNIED
GGVQLAYHYQQNTPIGDGPVLLPDNHYLSVQSKLSKDPNEKRDHMVLLEFVTAAGITLGMDELYKGGTGG
SMVSKGEELFTGVVPILVELDGDVNGHKESVSGEGEGDATYGKLTLKFICTTGKLPVPWPTLVTTLTYGV
QCFSRYPDHMKQHDFFKSAMPEGYIQERTIFFKDDGNYKTRAEVKFEGDTLVNRIELKGIDFKEDGNILG
HKLEYNASAGVEDRGVMTAFMQALGFTAQQGGWEKC
6xHistidine-TEV-PanZ(D104)-AS-cpGFP-AS-(R105)PanZ Amino Acid Sequence
SEQ ID NO: 48
MHHHHHHENLYFQSMKLTIIRLEKESDQDRIDLQKIWPEYSPSSLQVDDNHRIYAARFNERLLAAVRVTL
SGTEGALDSLRVREVTRRRGVGQYLLEEVLENNPGVSCWWMADAGVEDASNVYIKADKQKNGIKANFKIR
HNIEDGGVQLAYHYQQNTPIGDGPVLLPDNHYLSVQSKLSKDPNEKRDHMVLLEFVTAAGITLGMDELYK
GGTGGSMVSKGEELFTGVVPILVELDGDVNGHKFSVSGEGEGDATYGKLTLKFICTTGKLPVPWPTLVTT
LTYGVQCFSRYPDHMKQHDFFKSAMPEGYIQERTIFFKDDGNYKTRAEVKFEGDTLVNRIELKGIDFKED
GNILGHKLEYNASRGVMTAFMQALGFTAQQGGWEKC
6xHistidine-TEV-PanZ(G116)-AS-cpGFP-AS-(F117)PanZ Amino Acid Sequence
SEQ ID NO: 49
MHHHHHHENLYFQSMKLTIIRLEKESDQDRIDLQKIWPEYSPSSLQVDDNHRIYAARENERLLAAVRVTL
SGTEGALDSLRVREVTRRRGVGQYLLEEVLRNNPGVSCWWMADAGVEDRGVMTAFMQALGASNVYIKADK
QKNGIKANFKIRHNIEDGGVQLAYHYQQNTPIGDGPVLLPDNHYLSVQSKLSKDPNEKRDHMVLLEFVTA
AGITLGMDELYKGGTGGSMVSKGEELFTGVVPILVELDGDVNGHKESVSGEGEGDATYGKLTLKFICTTG
KLPVPWPTLVTTLTYGVQCFSRYPDHMKQHDFFKSAMPEGYIQERTIFFKDDGNYKTRAEVKFEGDTLVN
RIELKGIDFKEDGNILGHKLEYNASFTAQQGGWEKC
6xHistidine-TEV-PanZ(R69)-GAS-cpGFP-G-(E70)PanZ Amino Acid Sequence
SEQ ID NO: 50
KLTIIRLEKESDQDRIDLQKIWPEYSPSSLQVDDNHRIYAARFNERLLAAVRVTLSGTEGALDSLRVRGA
SNVYIKADKQKNGIKANFKIRHNIEDGGVQLAYHYQQNTPIGDGPVLLPDNHYLSVQSKLSKDPNEKRDH
MVLLEFVTAAGITLGMDELYKGGTGGSMVSKGEELFTGVVPILVELDGDVNGHKESVSGEGEGDATYGKL
TLKFICTTGKLPVPWPTLVTTLTYGVQCFSRYPDHMKQHDFFKSAMPEGYIQERTIFFKDDGNYKTRAEV
KFEGDTLVNRIELKGIDFKEDGNILGHKLEYNGEVTRRRGVGQYLLEEVLRNNPGVSCWWMADAGVEDRG
VMTAFMQALGFTAQQGGWEKC
6xHistidine-TEV-PanZ(R69)-GAS-cpGFP-(E70)PanZ Amino Acid Sequence
SEQ ID NO: 51
MHHHHHHENLYFQSMKLTIIRLEKESDQDRIDLQKIWPEYSPSSLQVDDNHRIYAARFNERLLAAVRVTL
SGTEGALDSLRVRGASNVYIKADKQKNGIKANFKIRHNIEDGGVQLAYHYQQNTPIGDGPVLLPDNHYLS
VQSKLSKDPNEKRDHMVLLEFVTAAGITLGMDELYKGGTGGSMVSKGEELFTGVVPILVELDGDVNGHKE
SVSGEGEGDATYGKLTLKFICTTGKLPVPWPTLVTTLTYGVQCFSRYPDHMKQHDFFKSAMPEGYIQERT
IFFKDDGNYKTRAEVKFEGDTLVNRIELKGIDFKEDGNILGHKLEYNEVTRRRGVGQYLLEEVLRNNPGV
SCWWMADAGVEDRGVMTAFMQALGFTAQQGGWEKC
6xHistidine-TEV-PanZ(R69)-GA-cpGFP-(E70)PanZ Amino Acid Sequence
SEQ ID NO: 52
MHHHHHHENLYFQSMKLTIIRLEKESDQDRIDLQKIWPEYSPSSLQVDDNHRIYAARFNERLLAAVRVTL
SGTEGALDSLRVRGANVYIKADKQKNGIKANFKIRHNIEDGGVQLAYHYQQNTPIGDGPVLLPDNHYLSV
QSKLSKDPNEKRDHMVLLEFVTAAGITLGMDELYKGGTGGSMVSKGEELFTGVVPILVELDGDVNGHKES
VSGEGEGDATYGKLTLKFICTTGKLPVPWPTLVTTLTYGVQCFSRYPDHMKQHDFFKSAMPEGYIQERTI
FFKDDGNYKTRAEVKFEGDTLVNRIELKGIDFKEDGNILGHKLEYNEVTRRRGVGQYLLEEVLRNNPGVS
CWWMADAGVEDRGVMTAFMQALGFTAQQGGWEKC
6xHistidine-TEV-PanZ(R69)-cpGFP-(E70)PanZ Amino Acid Sequence
SEQ ID NO: 53
MHHHHHHENLYFQSMKLTIIRLEKESDQDRIDLQKIWPEYSPSSLQVDDNHRIYAARFNERLLAAVRVTL
SGTEGALDSLRVRNVYIKADKQKNGIKANFKIRHNIEDGGVQLAYHYQQNTPIGDGPVLLPDNHYLSVQS
KLSKDPNEKRDHMVLLEFVTAAGITLGMDELYKGGTGGSMVSKGEELFTGVVPILVELDGDVNGHKESVS
GEGEGDATYGKLTLKFICTTGKLPVPWPTLVTTLTYGVQCFSRYPDHMKQHDFFKSAMPEGYIQERTIFF
KDDGNYKTRAEVKFEGDTLVNRIELKGIDFKEDGNILGHKLEYNEVTRRRGVGQYLLEEVLRNNPGVSCW
WMADAGVEDRGVMTAFMQALGFTAQQGGWEKC
6xHistidine-TEV-PanZ(69)-G-cpGFP-GAS-(E70)PanZ Amino Acid Sequence
SEQ ID NO: 54
MHHHHHHENLYFQSMKLTIIRLEKESDQDRIDLQKIWPEYSPSSLQVDDNHRIYAARFNERLLAAVRVTL
SGTEGALDSLRVRGNVYIKADKQKNGIKANFKIRHNIEDGGVQLAYHYQQNTPIGDGPVLLPDNHYLSVQ
SKLSKDPNEKRDHMVLLEFVTAAGITLGMDELYKGGTGGSMVSKGEELFTGVVPILVELDGDVNGHKESV
SGEGEGDATYGKLTLKFICTTGKLPVPWPTLVTTLTYGVQCFSRYPDHMKQHDFFKSAMPEGYIQERTIF
FKDDGNYKTRAEVKFEGDTLVNRIELKGIDFKEDGNILGHKLEYNGASEVTRRRGVGQYLLEEVLRNNPG
VSCWWMADAGVEDRGVMTAFMQALGFTAQQGGWEKC
6xHistidine-TEV-PanZ(R69)-cpGFP-GAS-(E70)PanZ Amino Acid Sequence
SEQ ID NO: 55
MHHHHHHENLYFQSMKLTIIRLEKESDQDRIDLQKIWPEYSPSSLQVDDNHRIYAARFNERLLAAVRVTL
SGTEGALDSLRVRNVYIKADKQKNGIKANFKIRHNIEDGGVQLAYHYQQNTPIGDGPVLLPDNHYLSVQS
KLSKDPNEKRDHMVLLEFVTAAGITLGMDELYKGGTGGSMVSKGEELFTGVVPILVELDGDVNGHKESVS
GEGEGDATYGKLTLKFICTTGKLPVPWPTLVTTLTYGVQCFSRYPDHMKQHDFFKSAMPEGYIQERTIFF
KDDGNYKTRAEVKFEGDTLVNRIELKGIDFKEDGNILGHKLEYNGASEVTRRRGVGQYLLEEVLRNNPGV
SCWWMADAGVEDRGVMTAFMQALGFTAQQGGWEKC
6xHistidine-TEV-PanZ(R69)-GA-cpYFP-GA-(E70)PanZ Amino Acid Sequence
SEQ ID NO: 56
MHHHHHHENLYFQSMKLTIIRLEKFSDQDRIDLQKIWPEYSPSSLQVDDNHRIYAARFNERLLAAVRVTL
SGTEGALDSLRVRGANVYIKADKQKNGIKANFKIRHNIEDGGVQLAYHYQQNTPIGDGPVLLPDNHYLSY
QSVLSKDPNEKRDHMVLLEFVTAAGITLGMDELYKGGTGGSMVSKGEELFTGVVPILVELDGDVNGHKES
VSGEGEGDATYGKLTLKFICTTGKLPVPWPTLVTTLTYGVQCFSRYPDHMKQHDFFKSAMPEGYIQERTI
FFKDDGNYKTRAEVKFEGDTLVNRIELKGIDFKEDGNILGHKLEYNGAEVTRRRGVGQYLLEEVLRNNPG
VSCWWMADAGVEDRGVMTAFMQALGFTAQQGGWEKC
6xHistidine-TEV-PanZ(R69)-GA-cpBFP-GA-(E70)PanZ Amino Acid Sequence
SEQ ID NO: 57
MHHHHHHENLYFQSMKLTIIRLEKESDQDRIDLQKIWPEYSPSSLQVDDNHRIYAARFNERLLAAVRVTL
SGTEGALDSLRVRGANVYIKADKQKNGIKANFKIRHNIEDGGVQLAYHYQQNTPIGDGPVLLPDNHYLSV
QSKLSKDPNEKRDHMVLLEFVTAAGITLGMDELYKGGTGGSMVSKGEELFTGVVPILVELDGDVNGHKES
VSGEGEGDATYGKLTLKFICTTGKLPVPWPTLVTTLSHGVQCFSRYPDHMKQHDFFKSAMPEGYIQERTI
FFKDDGNYKTRAEVKFEGDTLVNRIELKGIDFKEDGNILGHKLEYNGAEVTRRRGVGQYLLEEVLRNNPG
VSCWWMADAGVEDRGVMTAFMQALGFTAQQGGWEKC
6xHistidine-TEV-PanZ Nucleic Acid Sequence
SEQ ID NO: 58
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAGAGGTGACAAGACGGAGAGGGGTAGGACAAT
ATTTGCTCGAGGAGGTCCTTAGAAATAACCCCGGAGTAAGTTGCTGGTGGATGGCGGACGCTGGAGTTGA
GGATCGCGGAGTCATGACGGCTTTCATGCAGGCCCTTGGGTTCACCGCACAGCAAGGGGGCTGGGAGAAG
TGC
6xHistidine-TEV-cpGFP Nucleic Acid Sequence
SEQ ID NO: 59
ATGCATCACCATCACCATCACGAAAACCTGTACTTCCAAAGCAACGTCTATATCAAGGCCGACAAGCAGA
AGAACGGCATCAAGGCGAACTTCAAGATCCGCCACAACATCGAGGACGGCGGCGTGCAGCTCGCCTACCA
CTACCAGCAGAACACCCCCATCGGCGACGGCCCCGTGCTGCTGCCCGACAACCACTACCTGAGCGTGCAG
TCCAAACTTTCGAAAGACCCCAACGAGAAGCGCGATCACATGGTCCTGCTGGAGTTCGTGACCGCCGCCG
GGATCACTCTCGGCATGGACGAGCTGTACAAGGGCGGTACCGGAGGGAGCATGGTGAGCAAGGGCGAGGA
GCTGTTCACCGGGGTGGTGCCCATCCTGGTCGAGCTGGACGGCGACGTAAACGGCCACAAGTTCAGCGTG
TCCGGCGAGGGTGAGGGCGATGCCACCTACGGCAAGCTGACCCTGAAGTTCATCTGCACCACCGGCAAGC
TGCCCGTGCCCTGGCCCACCCTCGTGACCACCCTGACCTACGGCGTGCAGTGCTTCAGCCGCTACCCCGA
CCACATGAAGCAGCACGACTTCTTCAAGTCCGCCATGCCCGAAGGCTACATCCAGGAGCGCACCATCTTC
TTCAAGGACGACGGCAACTACAAGACCCGCGCCGAGGTGAAGTTCGAGGGCGACACCCTGGTGAACCGCA
TCGAGCTGAAGGGCATCGACTTCAAGGAGGACGGCAACATCCTGGGGCACAAGCTGGAGTACAAC
6xHistidine-TEV-cpYFP Nucleic Acid Sequence
SEQ ID NO: 60
ATGCATCACCATCACCATCACGAAAACCTGTACTTCCAAAGCAACGTCTATATCAAGGCCGACAAGCAGA
AGAACGGCATCAAGGCGAACTTCAAGATCCGCCACAACATCGAGGACGGCGGCGTGCAGCTCGCCTACCA
CTACCAGCAGAACACCCCCATCGGCGACGGCCCCGTGCTGCTGCCCGACAACCACTACCTGAGCTATCAG
TCCGTACTTTCGAAAGACCCCAACGAGAAGCGCGATCACATGGTCCTGCTGGAGTTCGTGACCGCCGCCG
GGATCACTCTCGGCATGGACGAGCTGTACAAGGGCGGTACCGGAGGGAGCATGGTGAGCAAGGGCGAGGA
GCTGTTCACCGGGGTGGTGCCCATCCTGGTCGAGCTGGACGGCGACGTAAACGGCCACAAGTTCAGCGTG
TCCGGCGAGGGTGAGGGCGATGCCACCTACGGCAAGCTGACCCTGAAGTTCATCTGCACCACCGGCAAGC
TGCCCGTGCCCTGGCCCACCCTCGTGACCACCCTGACCTACGGCGTGCAGTGCTTCAGCCGCTACCCCGA
CCACATGAAGCAGCACGACTTCTTCAAGTCCGCCATGCCCGAAGGCTACATCCAGGAGCGCACCATCTTC
TTCAAGGACGACGGCAACTACAAGACCCGCGCCGAGGTGAAGTTCGAGGGCGACACCCTGGTGAACCGCA
TCGAGCTGAAGGGCATCGACTTCAAGGAGGACGGCAACATCCTGGGGCACAAGCTGGAGTACAAC
6xHistidine-TEV-cpBFP Nucleic Acid Sequence
SEQ ID NO: 61
ATGCATCACCATCACCATCACGAAAACCTGTACTTCCAAAGCAACGTCTATATCAAGGCCGACAAGCAGA
AGAACGGCATCAAGGCGAACTTCAAGATCCGCCACAACATCGAGGACGGCGGCGTGCAGCTCGCCTACCA
CTACCAGCAGAACACCCCCATCGGCGACGGCCCCGTGCTGCTGCCCGACAACCACTACCTGAGCGTGCAG
TCCAAACTTTCGAAAGACCCCAACGAGAAGCGCGATCACATGGTCCTGCTGGAGTTCGTGACCGCCGCCG
GGATCACTCTCGGCATGGACGAGCTGTACAAGGGCGGTACCGGAGGGAGCATGGTGAGCAAGGGCGAGGA
GCTGTTCACCGGGGTGGTGCCCATCCTGGTCGAGCTGGACGGCGACGTAAACGGCCACAAGTTCAGCGTG
TCCGGCGAGGGTGAGGGCGATGCCACCTACGGCAAGCTGACCCTGAAGTTCATCTGCACCACCGGCAAGC
TGCCCGTGCCCTGGCCCACCCTCGTGACCACCCTGTCCCACGGCGTGCAGTGCTTCAGCCGCTACCCCGA
CCACATGAAGCAGCACGACTTCTTCAAGTCCGCCATGCCCGAAGGCTACATCCAGGAGCGCACCATCTTC
TTCAAGGACGACGGCAACTACAAGACCCGCGCCGAGGTGAAGTTCGAGGGCGACACCCTGGTGAACCGCA
TCGAGCTGAAGGGCATCGACTTCAAGGAGGACGGCAACATCCTGGGGCACAAGCTGGAGTACAAC
6xHistidine-TEV-PanZ(R69)-GA-cpGFP-GA-(E70)PanZ Nucleic Acid Sequence
SEQ ID NO: 62
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAGGAGCAAACGTCTATATCAAGGCCGACAAGC
AGAAGAACGGCATCAAGGCGAACTTCAAGATCCGCCACAACATCGAGGACGGCGGCGTGCAGCTCGCCTA
CCACTACCAGCAGAACACCCCCATCGGCGACGGCCCCGTGCTGCTGCCCGACAACCACTACCTGAGCGTG
CAGTCCAAACTTTCGAAAGACCCCAACGAGAAGCGCGATCACATGGTCCTGCTGGAGTTCGTGACCGCCG
CCGGGATCACTCTCGGCATGGACGAGCTGTACAAGGGCGGTACCGGAGGGAGCATGGTGAGCAAGGGCGA
GGAGCTGTTCACCGGGGTGGTGCCCATCCTGGTCGAGCTGGACGGCGACGTAAACGGCCACAAGTTCAGC
GTGTCCGGCGAGGGTGAGGGCGATGCCACCTACGGCAAGCTGACCCTGAAGTTCATCTGCACCACCGGCA
AGCTGCCCGTGCCCTGGCCCACCCTCGTGACCACCCTGACCTACGGCGTGCAGTGCTTCAGCCGCTACCC
CGACCACATGAAGCAGCACGACTTCTTCAAGTCCGCCATGCCCGAAGGCTACATCCAGGAGCGCACCATC
TTCTTCAAGGACGACGGCAACTACAAGACCCGCGCCGAGGTGAAGTTCGAGGGCGACACCCTGGTGAACC
GCATCGAGCTGAAGGGCATCGACTTCAAGGAGGACGGCAACATCCTGGGGCACAAGCTGGAGTACAACGG
TGCTGAGGTGACAAGACGGAGAGGGGTAGGACAATATTTGCTCGAGGAGGTCCTTAGAAATAACCCCGGA
GTAAGTTGCTGGTGGATGGCGGACGCTGGAGTTGAGGATCGCGGAGTCATGACGGCTTTCATGCAGGCCC
TTGGGTTCACCGCACAGCAAGGGGGCTGGGAGAAGTGC
6xHistidine-TEV-PanZ(W23)-AS-cpGFP-AS-(P24)PanZ Nucleic Acid Sequence
SEQ ID NO: 63
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGGCATCTAACGTCTATATCAAGGCCGACAA
GCAGAAGAACGGCATCAAGGCGAACTTCAAGATCCGCCACAACATCGAGGACGGCGGCGTGCAGCTCGCC
TACCACTACCAGCAGAACACCCCCATCGGCGACGGCCCCGTGCTGCTGCCCGACAACCACTACCTGAGCG
TGCAGTCCAAACTTTCGAAAGACCCCAACGAGAAGCGCGATCACATGGTCCTGCTGGAGTTCGTGACCGC
CGCCGGGATCACTCTCGGCATGGACGAGCTGTACAAGGGCGGTACCGGAGGGAGCATGGTGAGCAAGGGC
GAGGAGCTGTTCACCGGGGTGGTGCCCATCCTGGTCGAGCTGGACGGCGACGTAAACGGCCACAAGTTCA
GCGTGTCCGGCGAGGGTGAGGGCGATGCCACCTACGGCAAGCTGACCCTGAAGTTCATCTGCACCACCGG
CAAGCTGCCCGTGCCCTGGCCCACCCTCGTGACCACCCTGACCTACGGCGTGCAGTGCTTCAGCCGCTAC
CCCGACCACATGAAGCAGCACGACTTCTTCAAGTCCGCCATGCCCGAAGGCTACATCCAGGAGCGCACCA
TCTTCTTCAAGGACGACGGCAACTACAAGACCCGCGCCGAGGTGAAGTTCGAGGGCGACACCCTGGTGAA
CCGCATCGAGCTGAAGGGCATCGACTTCAAGGAGGACGGCAACATCCTGGGGCACAAGCTGGAGTACAAC
GCGTCACCCGAATATTCTCCCAGTAGCCTCCAAGTAGACGACAACCATCGAATCTACGCCGCGAGATTTA
ATGAACGACTGCTTGCGGCGGTTAGGGTAACCCTCAGCGGTACGGAAGGTGCTCTGGACTCCCTGCGGGT
CCGAGAGGTGACAAGACGGAGAGGGGTAGGACAATATTTGCTCGAGGAGGTCCTTAGAAATAACCCCGGA
GTAAGTTGCTGGTGGATGGCGGACGCTGGAGTTGAGGATCGCGGAGTCATGACGGCTTTCATGCAGGCCC
TTGGGTTCACCGCACAGCAAGGGGGCTGGGAGAAGTGC
6xHistidine-TEV-PanZ(V71)-AS-cpGFP-AS-(T72)PanZ Nucleic Acid Sequence
SEQ ID NO: 64
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAGAGGTGGCATCTAACGTCTATATCAAGGCCG
ACAAGCAGAAGAACGGCATCAAGGCGAACTTCAAGATCCGCCACAACATCGAGGACGGCGGCGTGCAGCT
CGCCTACCACTACCAGCAGAACACCCCCATCGGCGACGGCCCCGTGCTGCTGCCCGACAACCACTACCTG
AGCGTGCAGTCCAAACTTTCGAAAGACCCCAACGAGAAGCGCGATCACATGGTCCTGCTGGAGTTCGTGA
CCGCCGCCGGGATCACTCTCGGCATGGACGAGCTGTACAAGGGCGGTACCGGAGGGAGCATGGTGAGCAA
GGGCGAGGAGCTGTTCACCGGGGTGGTGCCCATCCTGGTCGAGCTGGACGGCGACGTAAACGGCCACAAG
TTCAGCGTGTCCGGCGAGGGTGAGGGCGATGCCACCTACGGCAAGCTGACCCTGAAGTTCATCTGCACCA
CCGGCAAGCTGCCCGTGCCCTGGCCCACCCTCGTGACCACCCTGACCTACGGCGTGCAGTGCTTCAGCCG
CTACCCCGACCACATGAAGCAGCACGACTTCTTCAAGTCCGCCATGCCCGAAGGCTACATCCAGGAGCGC
ACCATCTTCTTCAAGGACGACGGCAACTACAAGACCCGCGCCGAGGTGAAGTTCGAGGGCGACACCCTGG
TGAACCGCATCGAGCTGAAGGGCATCGACTTCAAGGAGGACGGCAACATCCTGGGGCACAAGCTGGAGTA
CAACGCGTCAACAAGACGGAGAGGGGTAGGACAATATTTGCTCGAGGAGGTCCTTAGAAATAACCCCGGA
GTAAGTTGCTGGTGGATGGCGGACGCTGGAGTTGAGGATCGCGGAGTCATGACGGCTTTCATGCAGGCCC
TTGGGTTCACCGCACAGCAAGGGGGCTGGGAGAAGTGC
6xHistidine-TEV-PanZ(R69)-AS-cpGFP-AS-(E70)PanZ Nucleic Acid Sequence
SEQ ID NO: 65
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAGCATCTAACGTCTATATCAAGGCCGACAAGC
AGAAGAACGGCATCAAGGCGAACTTCAAGATCCGCCACAACATCGAGGACGGCGGCGTGCAGCTCGCCTA
CCACTACCAGCAGAACACCCCCATCGGCGACGGCCCCGTGCTGCTGCCCGACAACCACTACCTGAGCGTG
CAGTCCAAACTTTCGAAAGACCCCAACGAGAAGCGCGATCACATGGTCCTGCTGGAGTTCGTGACCGCCG
CCGGGATCACTCTCGGCATGGACGAGCTGTACAAGGGCGGTACCGGAGGGAGCATGGTGAGCAAGGGCGA
GGAGCTGTTCACCGGGGTGGTGCCCATCCTGGTCGAGCTGGACGGCGACGTAAACGGCCACAAGTTCAGC
GTGTCCGGCGAGGGTGAGGGCGATGCCACCTACGGCAAGCTGACCCTGAAGTTCATCTGCACCACCGGCA
AGCTGCCCGTGCCCTGGCCCACCCTCGTGACCACCCTGACCTACGGCGTGCAGTGCTTCAGCCGCTACCC
CGACCACATGAAGCAGCACGACTTCTTCAAGTCCGCCATGCCCGAAGGCTACATCCAGGAGCGCACCATC
TTCTTCAAGGACGACGGCAACTACAAGACCCGCGCCGAGGTGAAGTTCGAGGGCGACACCCTGGTGAACC
GCATCGAGCTGAAGGGCATCGACTTCAAGGAGGACGGCAACATCCTGGGGCACAAGCTGGAGTACAACGC
GTCAGAGGTGACAAGACGGAGAGGGGTAGGACAATATTTGCTCGAGGAGGTCCTTAGAAATAACCCCGGA
GTAAGTTGCTGGTGGATGGCGGACGCTGGAGTTGAGGATCGCGGAGTCATGACGGCTTTCATGCAGGCCC
TTGGGTTCACCGCACAGCAAGGGGGCTGGGAGAAGTGC
6xHistidine-TEV-PanZ(D99)-AS-cpGFP-AS-(A100)PanZ Nucleic Acid Sequence
SEQ ID NO: 66
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAGAGGTGACAAGACGGAGAGGGGTAGGACAAT
ATTTGCTCGAGGAGGTCCTTAGAAATAACCCCGGAGTAAGTTGCTGGTGGATGGCGGACGCATCTAACGT
CTATATCAAGGCCGACAAGCAGAAGAACGGCATCAAGGCGAACTTCAAGATCCGCCACAACATCGAGGAC
GGCGGCGTGCAGCTCGCCTACCACTACCAGCAGAACACCCCCATCGGCGACGGCCCCGTGCTGCTGCCCG
ACAACCACTACCTGAGCGTGCAGTCCAAACTTTCGAAAGACCCCAACGAGAAGCGCGATCACATGGTCCT
GCTGGAGTTCGTGACCGCCGCCGGGATCACTCTCGGCATGGACGAGCTGTACAAGGGCGGTACCGGAGGG
AGCATGGTGAGCAAGGGCGAGGAGCTGTTCACCGGGGTGGTGCCCATCCTGGTCGAGCTGGACGGCGACG
TAAACGGCCACAAGTTCAGCGTGTCCGGCGAGGGTGAGGGCGATGCCACCTACGGCAAGCTGACCCTGAA
GTTCATCTGCACCACCGGCAAGCTGCCCGTGCCCTGGCCCACCCTCGTGACCACCCTGACCTACGGCGTG
CAGTGCTTCAGCCGCTACCCCGACCACATGAAGCAGCACGACTTCTTCAAGTCCGCCATGCCCGAAGGCT
ACATCCAGGAGCGCACCATCTTCTTCAAGGACGACGGCAACTACAAGACCCGCGCCGAGGTGAAGTTCGA
GGGCGACACCCTGGTGAACCGCATCGAGCTGAAGGGCATCGACTTCAAGGAGGACGGCAACATCCTGGGG
CACAAGCTGGAGTACAACGCGTCAGCTGGAGTTGAGGATCGCGGAGTCATGACGGCTTTCATGCAGGCCC
TTGGGTTCACCGCACAGCAAGGGGGCTGGGAGAAGTGC
6xHistidine-TEV-PanZ(D104)-AS-cpGFP-AS-(R105)PanZ Nucleic Acid Sequence
SEQ ID NO: 67
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAGAGGTGACAAGACGGAGAGGGGTAGGACAAT
ATTTGCTCGAGGAGGTCCTTAGAAATAACCCCGGAGTAAGTTGCTGGTGGATGGCGGACGCTGGAGTTGA
GGATGCATCTAACGTCTATATCAAGGCCGACAAGCAGAAGAACGGCATCAAGGCGAACTTCAAGATCCGC
CACAACATCGAGGACGGCGGCGTGCAGCTCGCCTACCACTACCAGCAGAACACCCCCATCGGCGACGGCC
CCGTGCTGCTGCCCGACAACCACTACCTGAGCGTGCAGTCCAAACTTTCGAAAGACCCCAACGAGAAGCG
CGATCACATGGTCCTGCTGGAGTTCGTGACCGCCGCCGGGATCACTCTCGGCATGGACGAGCTGTACAAG
GGCGGTACCGGAGGGAGCATGGTGAGCAAGGGCGAGGAGCTGTTCACCGGGGTGGTGCCCATCCTGGTCG
AGCTGGACGGCGACGTAAACGGCCACAAGTTCAGCGTGTCCGGCGAGGGTGAGGGCGATGCCACCTACGG
CAAGCTGACCCTGAAGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACGACTTCTTCAAGTCCG
CCATGCCCGAAGGCTACATCCAGGAGCGCACCATCTTCTTCAAGGACGACGGCAACTACAAGACCCGCGC
CGAGGTGAAGTTCGAGGGCGACACCCTGGTGAACCGCATCGAGCTGAAGGGCATCGACTTCAAGGAGGAC
GGCAACATCCTGGGGCACAAGCTGGAGTACAACGCGTCACGCGGAGTCATGACGGCTTTCATGCAGGCCC
TTGGGTTCACCGCACAGCAAGGGGGCTGGGAGAAGTGC
6xHistidine-TEV-PanZ(G116)-AS-cpGFP-AS-(F117)PanZ Nucleic Acid Sequence
SEQ ID NO: 68
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAGAGGTGACAAGACGGAGAGGGGTAGGACAAT
ATTTGCTCGAGGAGGTCCTTAGAAATAACCCCGGAGTAAGTTGCTGGTGGATGGCGGACGCTGGAGTTGA
GGATCGCGGAGTCATGACGGCTTTCATGCAGGCCCTTGGGGCATCTAACGTCTATATCAAGGCCGACAAG
CAGAAGAACGGCATCAAGGCGAACTTCAAGATCCGCCACAACATCGAGGACGGCGGCGTGCAGCTCGCCT
ACCACTACCAGCAGAACACCCCCATCGGCGACGGCCCCGTGCTGCTGCCCGACAACCACTACCTGAGCGT
GCAGTCCAAACTTTCGAAAGACCCCAACGAGAAGCGCGATCACATGGTCCTGCTGGAGTTCGTGACCGCC
GCCGGGATCACTCTCGGCATGGACGAGCTGTACAAGGGCGGTACCGGAGGGAGCATGGTGAGCAAGGGCG
AGGAGCTGTTCACCGGGGTGGTGCCCATCCTGGTCGAGCTGGACGGCGACGTAAACGGCCACAAGTTCAG
CGTGTCCGGCGAGGGTGAGGGCGATGCCACCTACGGCAAGCTGACCCTGAAGTTCATCTGCACCACCGGC
AAGCTGCCCGTGCCCTGGCCCACCCTCGTGACCACCCTGACCTACGGCGTGCAGTGCTTCAGCCGCTACC
CCGACCACATGAAGCAGCACGACTTCTTCAAGTCCGCCATGCCCGAAGGCTACATCCAGGAGCGCACCAT
CTTCTTCAAGGACGACGGCAACTACAAGACCCGCGCCGAGGTGAAGTTCGAGGGCGACACCCTGGTGAAC
CGCATCGAGCTGAAGGGCATCGACTTCAAGGAGGACGGCAACATCCTGGGGCACAAGCTGGAGTACAACG
CGTCATTCACCGCACAGCAAGGGGGCTGGGAGAAGTGC
6xHistidine-TEV-PanZ(R69)-GAS-cpGFP-G-(E70)PanZ Nucleic Acid Sequence
SEQ ID NO: 69
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAGGAGCAAGTAACGTCTATATCAAGGCCGACA
AGCAGAAGAACGGCATCAAGGCGAACTTCAAGATCCGCCACAACATCGAGGACGGCGGCGTGCAGCTCGC
CTACCACTACCAGCAGAACACCCCCATCGGCGACGGCCCCGTGCTGCTGCCCGACAACCACTACCTGAGC
GTGCAGTCCAAACTTTCGAAAGACCCCAACGAGAAGCGCGATCACATGGTCCTGCTGGAGTTCGTGACCG
CCGCCGGGATCACTCTCGGCATGGACGAGCTGTACAAGGGCGGTACCGGAGGGAGCATGGTGAGCAAGGG
CGAGGAGCTGTTCACCGGGGTGGTGCCCATCCTGGTCGAGCTGGACGGCGACGTAAACGGCCACAAGTTC
AGCGTGTCCGGCGAGGGTGAGGGCGATGCCACCTACGGCAAGCTGACCCTGAAGTTCATCTGCACCACCG
GCAAGCTGCCCGTGCCCTGGCCCACCCTCGTGACCACCCTGACCTACGGCGTGCAGTGCTTCAGCCGCTA
CCCCGACCACATGAAGCAGCACGACTTCTTCAAGTCCGCCATGCCCGAAGGCTACATCCAGGAGCGCACC
ATCTTCTTCAAGGACGACGGCAACTACAAGACCCGCGCCGAGGTGAAGTTCGAGGGCGACACCCTGGTGA
ACCGCATCGAGCTGAAGGGCATCGACTTCAAGGAGGACGGCAACATCCTGGGGCACAAGCTGGAGTACAA
CGGTGAGGTGACAAGACGGAGAGGGGTAGGACAATATTTGCTCGAGGAGGTCCTTAGAAATAACCCCGGA
GTAAGTTGCTGGTGGATGGCGGACGCTGGAGTTGAGGATCGCGGAGTCATGACGGCTTTCATGCAGGCCC
TTGGGTTCACCGCACAGCAAGGGGGCTGGGAGAAGTGC
6xHistidine-TEV-PanZ(R69)-GAS-cpGFP-(E70)PanZ Nucleic Acid Sequence
SEQ ID NO: 70
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAGGAGCAAGTAACGTCTATATCAAGGCCGACA
AGCAGAAGAACGGCATCAAGGCGAACTTCAAGATCCGCCACAACATCGAGGACGGCGGCGTGCAGCTCGC
CTACCACTACCAGCAGAACACCCCCATCGGCGACGGCCCCGTGCTGCTGCCCGACAACCACTACCTGAGC
GTGCAGTCCAAACTTTCGAAAGACCCCAACGAGAAGCGCGATCACATGGTCCTGCTGGAGTTCGTGACCG
CCGCCGGGATCACTCTCGGCATGGACGAGCTGTACAAGGGCGGTACCGGAGGGAGCATGGTGAGCAAGGG
CGAGGAGCTGTTCACCGGGGTGGTGCCCATCCTGGTCGAGCTGGACGGCGACGTAAACGGCCACAAGTTC
AGCGTGTCCGGCGAGGGTGAGGGCGATGCCACCTACGGCAAGCTGACCCTGAAGTTCATCTGCACCACCG
GCAAGCTGCCCGTGCCCTGGCCCACCCTCGTGACCACCCTGACCTACGGCGTGCAGTGCTTCAGCCGCTA
CCCCGACCACATGAAGCAGCACGACTTCTTCAAGTCCGCCATGCCCGAAGGCTACATCCAGGAGCGCACC
ATCTTCTTCAAGGACGACGGCAACTACAAGACCCGCGCCGAGGTGAAGTTCGAGGGCGACACCCTGGTGA
ACCGCATCGAGCTGAAGGGCATCGACTTCAAGGAGGACGGCAACATCCTGGGGCACAAGCTGGAGTACAA
CGAGGTGACAAGACGGAGAGGGGTAGGACAATATTTGCTCGAGGAGGTCCTTAGAAATAACCCCGGAGTA
AGTTGCTGGTGGATGGCGGACGCTGGAGTTGAGGATCGCGGAGTCATGACGGCTTTCATGCAGGCCCTTG
GGTTCACCGCACAGCAAGGGGGCTGGGAGAAGTGC
6xHistidine-TEV-PanZ(R69)-GA-cpGFP-(E70)PanZ Nucleic Acid Sequence
SEQ ID NO: 71
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAGGAGCAAACGTCTATATCAAGGCCGACAAGC
AGAAGAACGGCATCAAGGCGAACTTCAAGATCCGCCACAACATCGAGGACGGCGGCGTGCAGCTCGCCTA
CCACTACCAGCAGAACACCCCCATCGGCGACGGCCCCGTGCTGCTGCCCGACAACCACTACCTGAGCGTG
CAGTCCAAACTTTCGAAAGACCCCAACGAGAAGCGCGATCACATGGTCCTGCTGGAGTTCGTGACCGCCG
CCGGGATCACTCTCGGCATGGACGAGCTGTACAAGGGCGGTACCGGAGGGAGCATGGTGAGCAAGGGCGA
GGAGCTGTTCACCGGGGTGGTGCCCATCCTGGTCGAGCTGGACGGCGACGTAAACGGCCACAAGTTCAGC
GTGTCCGGCGAGGGTGAGGGCGATGCCACCTACGGCAAGCTGACCCTGAAGTTCATCTGCACCACCGGCA
AGCTGCCCGTGCCCTGGCCCACCCTCGTGACCACCCTGACCTACGGCGTGCAGTGCTTCAGCCGCTACCC
CGACCACATGAAGCAGCACGACTTCTTCAAGTCCGCCATGCCCGAAGGCTACATCCAGGAGCGCACCATC
TTCTTCAAGGACGACGGCAACTACAAGACCCGCGCCGAGGTGAAGTTCGAGGGCGACACCCTGGTGAACC
GCATCGAGCTGAAGGGCATCGACTTCAAGGAGGACGGCAACATCCTGGGGCACAAGCTGGAGTACAACGA
GGTGACAAGACGGAGAGGGGTAGGACAATATTTGCTCGAGGAGGTCCTTAGAAATAACCCCGGAGTAAGT
TGCTGGTGGATGGCGGACGCTGGAGTTGAGGATCGCGGAGTCATGACGGCTTTCATGCAGGCCCTTGGGT
TCACCGCACAGCAAGGGGGCTGGGAGAAGTGC
6xHistidine-TEV-PanZ(R69)-cpGFP-(E70)PanZ Nucleic Acid Sequence
SEQ ID NO: 72
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAAACGTCTATATCAAGGCCGACAAGCAGAAGA
ACGGCATCAAGGCGAACTTCAAGATCCGCCACAACATCGAGGACGGCGGCGTGCAGCTCGCCTACCACTA
CCAGCAGAACACCCCCATCGGCGACGGCCCCGTGCTGCTGCCCGACAACCACTACCTGAGCGTGCAGTCC
AAACTTTCGAAAGACCCCAACGAGAAGCGCGATCACATGGTCCTGCTGGAGTTCGTGACCGCCGCCGGGA
TCACTCTCGGCATGGACGAGCTGTACAAGGGCGGTACCGGAGGGAGCATGGTGAGCAAGGGCGAGGAGCT
GTTCACCGGGGTGGTGCCCATCCTGGTCGAGCTGGACGGCGACGTAAACGGCCACAAGTTCAGCGTGTCC
GGCGAGGGTGAGGGCGATGCCACCTACGGCAAGCTGACCCTGAAGTTCATCTGCACCACCGGCAAGCTGC
CCGTGCCCTGGCCCACCCTCGTGACCACCCTGACCTACGGCGTGCAGTGCTTCAGCCGCTACCCCGACCA
CATGAAGCAGCACGACTTCTTCAAGTCCGCCATGCCCGAAGGCTACATCCAGGAGCGCACCATCTTCTTC
AAGGACGACGGCAACTACAAGACCCGCGCCGAGGTGAAGTTCGAGGGCGACACCCTGGTGAACCGCATCG
AGCTGAAGGGCATCGACTTCAAGGAGGACGGCAACATCCTGGGGCACAAGCTGGAGTACAACGAGGTGAC
AAGACGGAGAGGGGTAGGACAATATTTGCTCGAGGAGGTCCTTAGAAATAACCCCGGAGTAAGTTGCTGG
TGGATGGCGGACGCTGGAGTTGAGGATCGCGGAGTCATGACGGCTTTCATGCAGGCCCTTGGGTTCACCG
CACAGCAAGGGGGCTGGGAGAAGTGC
6xHistidine-TEV-PanZ(69)-G-cpGFP-GAS-(E70)PanZ Nucleic Acid Sequence
SEQ ID NO: 73
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAGGAAACGTCTATATCAAGGCCGACAAGCAGA
AGAACGGCATCAAGGCGAACTTCAAGATCCGCCACAACATCGAGGACGGCGGCGTGCAGCTCGCCTACCA
CTACCAGCAGAACACCCCCATCGGCGACGGCCCCGTGCTGCTGCCCGACAACCACTACCTGAGCGTGCAG
TCCAAACTTTCGAAAGACCCCAACGAGAAGCGCGATCACATGGTCCTGCTGGAGTTCGTGACCGCCGCCG
GGATCACTCTCGGCATGGACGAGCTGTACAAGGGCGGTACCGGAGGGAGCATGGTGAGCAAGGGCGAGGA
GCTGTTCACCGGGGTGGTGCCCATCCTGGTCGAGCTGGACGGCGACGTAAACGGCCACAAGTTCAGCGTG
TCCGGCGAGGGTGAGGGCGATGCCACCTACGGCAAGCTGACCCTGAAGTTCATCTGCACCACCGGCAAGC
TGCCCGTGCCCTGGCCCACCCTCGTGACCACCCTGACCTACGGCGTGCAGTGCTTCAGCCGCTACCCCGA
CCACATGAAGCAGCACGACTTCTTCAAGTCCGCCATGCCCGAAGGCTACATCCAGGAGCGCACCATCTTC
TTCAAGGACGACGGCAACTACAAGACCCGCGCCGAGGTGAAGTTCGAGGGCGACACCCTGGTGAACCGCA
TCGAGCTGAAGGGCATCGACTTCAAGGAGGACGGCAACATCCTGGGGCACAAGCTGGAGTACAACGGTGC
TTCAGAGGTGACAAGACGGAGAGGGGTAGGACAATATTTGCTCGAGGAGGTCCTTAGAAATAACCCCGGA
GTAAGTTGCTGGTGGATGGCGGACGCTGGAGTTGAGGATCGCGGAGTCATGACGGCTTTCATGCAGGCCC
TTGGGTTCACCGCACAGCAAGGGGGCTGGGAGAAGTGC
6xHistidine-TEV-PanZ(R69)-cpGFP-GAS-(E70)PanZ Nucleic Acid Sequence
SEQ ID NO: 74
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAAACGTCTATATCAAGGCCGACAAGCAGAAGA
ACGGCATCAAGGCGAACTTCAAGATCCGCCACAACATCGAGGACGGCGGCGTGCAGCTCGCCTACCACTA
CCAGCAGAACACCCCCATCGGCGACGGCCCCGTGCTGCTGCCCGACAACCACTACCTGAGCGTGCAGTCC
AAACTTTCGAAAGACCCCAACGAGAAGCGCGATCACATGGTCCTGCTGGAGTTCGTGACCGCCGCCGGGA
TCACTCTCGGCATGGACGAGCTGTACAAGGGCGGTACCGGAGGGAGCATGGTGAGCAAGGGCGAGGAGCT
GTTCACCGGGGTGGTGCCCATCCTGGTCGAGCTGGACGGCGACGTAAACGGCCACAAGTTCAGCGTGTCC
GGCGAGGGTGAGGGCGATGCCACCTACGGCAAGCTGACCCTGAAGTTCATCTGCACCACCGGCAAGCTGC
CCGTGCCCTGGCCCACCCTCGTGACCACCCTGACCTACGGCGTGCAGTGCTTCAGCCGCTACCCCGACCA
CATGAAGCAGCACGACTTCTTCAAGTCCGCCATGCCCGAAGGCTACATCCAGGAGCGCACCATCTTCTTC
AAGGACGACGGCAACTACAAGACCCGCGCCGAGGTGAAGTTCGAGGGCGACACCCTGGTGAACCGCATCG
AGCTGAAGGGCATCGACTTCAAGGAGGACGGCAACATCCTGGGGCACAAGCTGGAGTACAACGGTGCTTC
AGAGGTGACAAGACGGAGAGGGGTAGGACAATATTTGCTCGAGGAGGTCCTTAGAAATAACCCCGGAGTA
AGTTGCTGGTGGATGGCGGACGCTGGAGTTGAGGATCGCGGAGTCATGACGGCTTTCATGCAGGCCCTTG
GGTTCACCGCACAGCAAGGGGGCTGGGAGAAGTGC
6xHistidine-TEV-PanZ(R69)-GA-cpYFP-GA-(E70)PanZ Nucleic Acid Sequence
SEQ ID NO: 75
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAGGAGCAAACGTCTATATCAAGGCCGACAAGC
AGAAGAACGGCATCAAGGCGAACTTCAAGATCCGCCACAACATCGAGGACGGCGGCGTGCAGCTCGCCTA
CCACTACCAGCAGAACACCCCCATCGGCGACGGCCCCGTGCTGCTGCCCGACAACCACTACCTGAGCTAT
CAGTCCGTACTTTCGAAAGACCCCAACGAGAAGCGCGATCACATGGTCCTGCTGGAGTTCGTGACCGCCG
CCGGGATCACTCTCGGCATGGACGAGCTGTACAAGGGCGGTACCGGAGGGAGCATGGTGAGCAAGGGCGA
GGAGCTGTTCACCGGGGTGGTGCCCATCCTGGTCGAGCTGGACGGCGACGTAAACGGCCACAAGTTCAGC
GTGTCCGGCGAGGGTGAGGGCGATGCCACCTACGGCAAGCTGACCCTGAAGTTCATCTGCACCACCGGCA
AGCTGCCCGTGCCCTGGCCCACCCTCGTGACCACCCTGACCTACGGCGTGCAGTGCTTCAGCCGCTACCC
CGACCACATGAAGCAGCACGACTTCTTCAAGTCCGCCATGCCCGAAGGCTACATCCAGGAGCGCACCATC
TTCTTCAAGGACGACGGCAACTACAAGACCCGCGCCGAGGTGAAGTTCGAGGGCGACACCCTGGTGAACC
GCATCGAGCTGAAGGGCATCGACTTCAAGGAGGACGGCAACATCCTGGGGCACAAGCTGGAGTACAACGG
TGCTGAGGTGACAAGACGGAGAGGGGTAGGACAATATTTGCTCGAGGAGGTCCTTAGAAATAACCCCGGA
GTAAGTTGCTGGTGGATGGCGGACGCTGGAGTTGAGGATCGCGGAGTCATGACGGCTTTCATGCAGGCCC
TTGGGTTCACCGCACAGCAAGGGGGCTGGGAGAAGTGC
6xHistidine-TEV-PanZ(R69)-GA-cpBFP-GA-(E70)PanZ Nucleic Acid Sequence
SEQ ID NO: 76
ATGCATCACCATCACCATCACGAAAACCTGTACTTCCAAAGCATGAAGCTGACAATCATTCGCCTGGAGA
AATTTTCCGACCAGGATAGAATCGACCTTCAGAAGATCTGGCCCGAATATTCTCCCAGTAGCCTCCAAGT
AGACGACAACCATCGAATCTACGCCGCGAGATTTAATGAACGACTGCTTGCGGCGGTTAGGGTAACCCTC
AGCGGTACGGAAGGTGCTCTGGACTCCCTGCGGGTCCGAGGAGCAAACGTCTATATCAAGGCCGACAAGC
AGAAGAACGGCATCAAGGCGAACTTCAAGATCCGCCACAACATCGAGGACGGCGGCGTGCAGCTCGCCTA
CCACTACCAGCAGAACACCCCCATCGGCGACGGCCCCGTGCTGCTGCCCGACAACCACTACCTGAGCGTG
CAGTCCAAACTTTCGAAAGACCCCAACGAGAAGCGCGATCACATGGTCCTGCTGGAGTTCGTGACCGCCG
CCGGGATCACTCTCGGCATGGACGAGCTGTACAAGGGCGGTACCGGAGGGAGCATGGTGAGCAAGGGCGA
GGAGCTGTTCACCGGGGTGGTGCCCATCCTGGTCGAGCTGGACGGCGACGTAAACGGCCACAAGTTCAGC
GTGTCCGGCGAGGGTGAGGGCGATGCCACCTACGGCAAGCTGACCCTGAAGTTCATCTGCACCACCGGCA
AGCTGCCCGTGCCCTGGCCCACCCTCGTGACCACCCTGTCCCACGGCGTGCAGTGCTTCAGCCGCTACCC
CGACCACATGAAGCAGCACGACTTCTTCAAGTCCGCCATGCCCGAAGGCTACATCCAGGAGCGCACCATC
TTCTTCAAGGACGACGGCAACTACAAGACCCGCGCCGAGGTGAAGTTCGAGGGCGACACCCTGGTGAACC
GCATCGAGCTGAAGGGCATCGACTTCAAGGAGGACGGCAACATCCTGGGGCACAAGCTGGAGTACAACGG
TGCTGAGGTGACAAGACGGAGAGGGGTAGGACAATATTTGCTCGAGGAGGTCCTTAGAAATAACCCCGGA
GTAAGTTGCTGGTGGATGGCGGACGCTGGAGTTGAGGATCGCGGAGTCATGACGGCTTTCATGCAGGCCC
TTGGGTTCACCGCACAGCAAGGGGGCTGGGAGAAGTGC
PanZ(R69)-GA-cpGFP-GA-(E70)PanZ (“PANcACe”) with NLS
Amino Acid Sequence
SEQ ID NO: 77
MVTGPKKKRKVDYKDDDDKLDGGYPYDVPDYAARGYQTSLYKKAGSTMGHMKLTIIRLEKFSDQDRIDLQ
KIWPEYSPSSLQVDDNHRIYAARFNERLLAAVRVTLSGTEGALDSLRVRGANVYIKADKQKNGIKANFKI
RHNIEDGGVQLAYHYQQNTPIGDGPVLLPDNHYLSVQSKLSKDPNEKRDHMVLLEFVTAAGITLGMDELY
KGGTGGSMVSKGEELFTGVVPILVELDGDVNGHKESVSGEGEGDATYGKLTLKFICTTGKLPVPWPTLVT
TLTYGVQCFSRYPDHMKQHDFFKSAMPEGYIQERTIFFKDDGNYKTRAEVKFEGDTLVNRIELKGIDFKE
DGNILGHKLEYNGAEVTRRRGVGQYLLEEVLRNNPGVSCWWMADAGVEDRGVMTAFMQALGFTAQQGGWE
KC
cpGFP with NLS Amino Acid Sequence
SEQ ID NO: 78
MVTGPKKKRKVDYKDDDDKLDGGYPYDVPDYAARGYQTSLYKKAGSTMGHNVYIKADKQKNGIKANFKIR
HNIEDGGVQLAYHYQQNTPIGDGPVLLPDNHYLSVQSKLSKDPNEKRDHMVLLEFVTAAGITLGMDELYK
GGTGGSMVSKGEELFTGVVPILVELDGDVNGHKFSVSGEGEGDATYGKLTLKFICTTGKLPVPWPTLVTT
LTYGVQCFSRYPDHMKQHDFFKSAMPEGYIQERTIFFKDDGNYKTRAEVKFEGDTLVNRIELKGIDFKED
GNILGHKLEYN
PanZ(R69)-GA-cpGFP-GA-(E70)PanZ (“PANcACe”) with CLS Amino
Acid Sequence
SEQ ID NO: 79
MVTGLQKKLEELELDDYKDDDDKLDGGYPYDVPDYAARGYQTSLYKKAGSTMGHMKLTIIRLEKESDQDR
IDLQKIWPEYSPSSLQVDDNHRIYAARFNERLLAAVRVTLSGTEGALDSLRVRGANVYIKADKQKNGIKA
NEKIRHNIEDGGVQLAYHYQQNTPIGDGPVLLPDNHYLSVQSKLSKDPNEKRDHMVLLEFVTAAGITLGM
DELYKGGTGGSMVSKGEELFTGVVPILVELDGDVNGHKESVSGEGEGDATYGKLTLKFICTTGKLPVPWP
TLVTTLTYGVQCFSRYPDHMKQHDFFKSAMPEGYIQERTIFFKDDGNYKTRAEVKFEGDTLVNRIELKGI
DFKEDGNILGHKLEYNGAEVTRRRGVGQYLLEEVLRNNPGVSCWWMADAGVEDRGVMTAFMQALGETAQQ
GGWEKC
cpGFP with CLS Amino Acid Sequence
SEQ ID NO: 80
MVTGLQKKLEELELDDYKDDDDKLDGGYPYDVPDYAARGYQTSLYKKAGSTMGHNVYIKADKQKNGIKAN
FKIRHNIEDGGVQLAYHYQQNTPIGDGPVLLPDNHYLSVQSKLSKDPNEKRDHMVLLEFVTAAGITLGMD
ELYKGGTGGSMVSKGEELFTGVVPILVELDGDVNGHKFSVSGEGEGDATYGKLTLKFICTTGKLPVPWPT
LVTTLTYGVQCFSRYPDHMKQHDFFKSAMPEGYIQERTIFFKDDGNYKTRAEVKFEGDTLVNRIELKGID
FKEDGNILGHKLEYN
PanZ(R69)-GA-cpGFP-GA-(E70)PanZ (“PANcACe”) with MLS
Amino Acid Sequence
SEQ ID NO: 81
MVLATRVESLVGKRAISTSVCVRAHTGDYKDDDDKLDGGYPYDVPDYAARGYQTSLYKKAGSTMGHMKLT
IIRLEKFSDQDRIDLQKIWPEYSPSSLQVDDNHRIYAARFNERLLAAVRVTLSGTEGALDSLRVRGANVY
IKADKQKNGIKANFKIRHNIEDGGVQLAYHYQQNTPIGDGPVLLPDNHYLSVQSKLSKDPNEKRDHMVLL
EFVTAAGITLGMDELYKGGTGGSMVSKGEELFTGVVPILVELDGDVNGHKESVSGEGEGDATYGKLTLKF
ICTTGKLPVPWPTLVTTLTYGVQCFSRYPDHMKQHDFFKSAMPEGYIQERTIFFKDDGNYKTRAEVKFEG
DTLVNRIELKGIDFKEDGNILGHKLEYNGAEVTRRRGVGQYLLEEVLRNNPGVSCWWMADAGVEDRGVMT
AFMQALGFTAQQGGWEKC
cpGFP with MLS Amino Acid Sequence
SEQ ID NO: 82
MVLATRVESLVGKRAISTSVCVRAHTGDYKDDDDKLDGGYPYDVPDYAARGYQTSLYKKAGSTMGHNVYI
KADKQKNGIKANFKIRHNIEDGGVQLAYHYQQNTPIGDGPVLLPDNHYLSVQSKLSKDPNEKRDHMVLLE
FVTAAGITLGMDELYKGGTGGSMVSKGEELFTGVVPILVELDGDVNGHKFSVSGEGEGDATYGKLTLKFI
CTTGKLPVPWPTLVTTLTYGVQCFSRYPDHMKQHDFFKSAMPEGYIQERTIFFKDDGNYKTRAEVKFEGD
TLVNRIELKGIDFKEDGNILGHKLEYN
PanZ(R69)-GA-cpGFP-GA-(E70)PanZ (“PANcACe”) with NLS
Nucleic Acid Sequence
SEQ ID NO: 83
ATGGTGACCGGTCCAAAGAAGAAGCGTAAGGTAGACTACAAGGATGACGATGACAAGCTCGATGGAGGAT
ACCCATACGATGTTCCAGATTACGCTGCTCGAGGTTATCAAACAAGTTTGTACAAAAAAGCAGGCTCCAC
CATGGGGCATATGAAGCTGACAATCATTCGCCTGGAGAAATTTTCCGACCAGGATAGAATCGACCTTCAG
AAGATCTGGCCCGAATATTCTCCCAGTAGCCTCCAAGTAGACGACAACCATCGAATCTACGCCGCGAGAT
TTAATGAACGACTGCTTGCGGCGGTTAGGGTAACCCTCAGCGGTACGGAAGGTGCTCTGGACTCCCTGCG
GGTCCGAGGAGCAAACGTCTATATCAAGGCCGACAAGCAGAAGAACGGCATCAAGGCGAACTTCAAGATC
CGCCACAACATCGAGGACGGCGGCGTGCAGCTCGCCTACCACTACCAGCAGAACACCCCCATCGGCGACG
GCCCCGTGCTGCTGCCCGACAACCACTACCTGAGCgtgCAGTCCAAACTTTCGAAAGACCCCAACGAGAA
GCGCGATCACATGGTCCTGCTGGAGTTCGTGACCGCCGCCGGGATCACTCTCGGCATGGACGAGCTGTAC
AAGGGCGGTACCGGAGGGAGCATGGTGAGCAAGGGCGAGGAGCTGTTCACCGGGGTGGTGCCCATCCTGG
TCGAGCTGGACGGCGACGTAAACGGCCACAAGTTCAGCGTGTCCGGCGAGGGTGAGGGCGATGCCACCTA
CGGCAAGCTGACCCTGAAGTTCATCTGCACCACCGGCAAGCTGCCCGTGCCCTGGCCCACCCTCGTGACC
ACCCTGACCTACGGCGTGCAGTGCTTCAGCCGCTACCCCGACCACATGAAGCAGCACGACTTCTTCAAGT
CCGCCATGCCCGAAGGCTACATCCAGGAGCGCACCATCTTCTTCAAGGACGACGGCAACTACAAGACCCG
CGCCGAGGTGAAGTTCGAGGGCGACACCCTGGTGAACCGCATCGAGCTGAAGGGCATCGACTTCAAGGAG
GACGGCAACATCCTGGGGCACAAGCTGGAGTACAACGGTGCTGAGGTGACAAGACGGAGAGGGGTAGGAC
AATATTTGCTCGAGGAGGTCCTTAGAAATAACCCCGGAGTAAGTTGCTGGTGGATGGCGGACGCTGGAGT
TGAGGATCGCGGAGTCATGACGGCTTTCATGCAGGCCCTTGGGTTCACCGCACAGCAAGGGGGCTGGGAG
AAGTGCTAA
cpGFP with NLS Nucleic Acid Sequence
SEQ ID NO: 84
ATGGTGACCGGTCCAAAGAAGAAGCGTAAGGTAGACTACAAGGATGACGATGACAAGCTCGATGGAGGAT
ACCCATACGATGTTCCAGATTACGCTGCTCGAGGTTATCAAACAAGTTTGTACAAAAAAGCAGGCTCCAC
CATGGGGCATAACGTCTATATCAAGGCCGACAAGCAGAAGAACGGCATCAAGGCGAACTTCAAGATCCGC
CACAACATCGAGGACGGCGGCGTGCAGCTCGCCTACCACTACCAGCAGAACACCCCCATCGGCGACGGCC
CCGTGCTGCTGCCCGACAACCACTACCTGAGCgtgCAGTCCAAACTTTCGAAAGACCCCAACGAGAAGCG
CGATCACATGGTCCTGCTGGAGTTCGTGACCGCCGCCGGGATCACTCTCGGCATGGACGAGCTGTACAAG
GGCGGTACCGGAGGGAGCATGGTGAGCAAGGGCGAGGAGCTGTTCACCGGGGTGGTGCCCATCCTGGTCG
AGCTGGACGGCGACGTAAACGGCCACAAGTTCAGCGTGTCCGGCGAGGGTGAGGGCGATGCCACCTACGG
CAAGCTGACCCTGAAGTTCATCTGCACCACCGGCAAGCTGCCCGTGCCCTGGCCCACCCTCGTGACCACC
CTGACCTACGGCGTGCAGTGCTTCAGCCGCTACCCCGACCACATGAAGCAGCACGACTTCTTCAAGTCCG
CCATGCCCGAAGGCTACATCCAGGAGCGCACCATCTTCTTCAAGGACGACGGCAACTACAAGACCCGCGC
CGAGGTGAAGTTCGAGGGCGACACCCTGGTGAACCGCATCGAGCTGAAGGGCATCGACTTCAAGGAGGAC
GGCAACATCCTGGGGCACAAGCTGGAGTACAACTAA
PanZ(R69)-GA-cpGFP-GA-(E70)PanZ (“PANcACe”) with CLS Nucleic Acid Sequence
SEQ ID NO: 85
ATGGTGACCGGTCTGCAGAAAAAGCTGGAAGAGCTGGAACTGGACGACTACAAGGATGACGATGACAAGC
TCGATGGAGGATACCCATACGATGTTCCAGATTACGCTGCTCGAGGTTATCAAACAAGTTTGTACAAAAA
AGCAGGCTCCACCATGGGGCATATGAAGCTGACAATCATTCGCCTGGAGAAATTTTCCGACCAGGATAGA
ATCGACCTTCAGAAGATCTGGCCCGAATATTCTCCCAGTAGCCTCCAAGTAGACGACAACCATCGAATCT
ACGCCGCGAGATTTAATGAACGACTGCTTGCGGCGGTTAGGGTAACCCTCAGCGGTACGGAAGGTGCTCT
GGACTCCCTGCGGGTCCGAGGAGCAAACGTCTATATCAAGGCCGACAAGCAGAAGAACGGCATCAAGGCG
AACTTCAAGATCCGCCACAACATCGAGGACGGCGGCGTGCAGCTCGCCTACCACTACCAGCAGAACACCC
CCATCGGCGACGGCCCCGTGCTGCTGCCCGACAACCACTACCTGAGCgtqCAGTCCAAACTTTCGAAAGA
CCCCAACGAGAAGCGCGATCACATGGTCCTGCTGGAGTTCGTGACCGCCGCCGGGATCACTCTCGGCATG
GACGAGCTGTACAAGGGCGGTACCGGAGGGAGCATGGTGAGCAAGGGCGAGGAGCTGTTCACCGGGGTGG
TGCCCATCCTGGTCGAGCTGGACGGCGACGTAAACGGCCACAAGTTCAGCGTGTCCGGCGAGGGTGAGGG
CGATGCCACCTACGGCAAGCTGACCCTGAAGTTCATCTGCACCACCGGCAAGCTGCCCGTGCCCTGGCCC
ACCCTCGTGACCACCCTGACCTACGGCGTGCAGTGCTTCAGCCGCTACCCCGACCACATGAAGCAGCACG
ACTTCTTCAAGTCCGCCATGCCCGAAGGCTACATCCAGGAGCGCACCATCTTCTTCAAGGACGACGGCAA
CTACAAGACCCGCGCCGAGGTGAAGTTCGAGGGCGACACCCTGGTGAACCGCATCGAGCTGAAGGGCATC
GACTTCAAGGAGGACGGCAACATCCTGGGGCACAAGCTGGAGTACAACGGTGCTGAGGTGACAAGACGGA
GAGGGGTAGGACAATATTTGCTCGAGGAGGTCCTTAGAAATAACCCCGGAGTAAGTTGCTGGTGGATGGC
GGACGCTGGAGTTGAGGATCGCGGAGTCATGACGGCTTTCATGCAGGCCCTTGGGTTCACCGCACAGCAA
GGGGGCTGGGAGAAGTGCTAA
cpGFP with CLS Nucleic Acid Sequence
SEQ ID NO: 86
ATGGTGACCGGTCTGCAGAAAAAGCTGGAAGAGCTGGAACTGGACGACTACAAGGATGACGATGACAAGC
TCGATGGAGGATACCCATACGATGTTCCAGATTACGCTGCTCGAGGTTATCAAACAAGTTTGTACAAAAA
AGCAGGCTCCACCATGGGGCATAACGTCTATATCAAGGCCGACAAGCAGAAGAACGGCATCAAGGCGAAC
TTCAAGATCCGCCACAACATCGAGGACGGCGGCGTGCAGCTCGCCTACCACTACCAGCAGAACACCCCCA
TCGGCGACGGCCCCGTGCTGCTGCCCGACAACCACTACCTGAGCgtgCAGTCCAAACTTTCGAAAGACCC
CAACGAGAAGCGCGATCACATGGTCCTGCTGGAGTTCGTGACCGCCGCCGGGATCACTCTCGGCATGGAC
GAGCTGTACAAGGGCGGTACCGGAGGGAGCATGGTGAGCAAGGGCGAGGAGCTGTTCACCGGGGTGGTGC
CCATCCTGGTCGAGCTGGACGGCGACGTAAACGGCCACAAGTTCAGCGTGTCCGGCGAGGGTGAGGGCGA
TGCCACCTACGGCAAGCTGACCCTGAAGTTCATCTGCACCACCGGCAAGCTGCCCGTGCCCTGGCCCACC
CTCGTGACCACCCTGACCTACGGCGTGCAGTGCTTCAGCCGCTACCCCGACCACATGAAGCAGCACGACT
TCTTCAAGTCCGCCATGCCCGAAGGCTACATCCAGGAGCGCACCATCTTCTTCAAGGACGACGGCAACTA
CAAGACCCGCGCCGAGGTGAAGTTCGAGGGCGACACCCTGGTGAACCGCATCGAGCTGAAGGGCATCGAC
TTCAAGGAGGACGGCAACATCCTGGGGCACAAGCTGGAGTACAACTAA
PanZ(R69)-GA-cpGFP-GA-(E70)PanZ (“PANcACe”) with MLS Nucleic Acid Sequence
SEQ ID NO: 87
ATGGTGCTGGCCACCCGCGTGTTCAGCCTGGTGGGCAAGCGCGCCATCAGCACCAGCGTGTGCGTGCGCG
CCCACACCGGTGACTACAAGGATGACGATGACAAGCTCGATGGAGGATACCCATACGATGTTCCAGATTA
CGCTGCTCGAGGTTATCAAACAAGTTTGTACAAAAAAGCAGGCTCCACCATGGGGCATATGAAGCTGACA
ATCATTCGCCTGGAGAAATTTTCCGACCAGGATAGAATCGACCTTCAGAAGATCTGGCCCGAATATTCTC
CCAGTAGCCTCCAAGTAGACGACAACCATCGAATCTACGCCGCGAGATTTAATGAACGACTGCTTGCGGC
GGTTAGGGTAACCCTCAGCGGTACGGAAGGTGCTCTGGACTCCCTGCGGGTCCGAGGAGCAAACGTCTAT
ATCAAGGCCGACAAGCAGAAGAACGGCATCAAGGCGAACTTCAAGATCCGCCACAACATCGAGGACGGCG
GCGTGCAGCTCGCCTACCACTACCAGCAGAACACCCCCATCGGCGACGGCCCCGTGCTGCTGCCCGACAA
CCACTACCTGAGCgtgCAGTCCAAACTTTCGAAAGACCCCAACGAGAAGCGCGATCACATGGTCCTGCTG
GAGTTCGTGACCGCCGCCGGGATCACTCTCGGCATGGACGAGCTGTACAAGGGCGGTACCGGAGGGAGCA
TGGTGAGCAAGGGCGAGGAGCTGTTCACCGGGGTGGTGCCCATCCTGGTCGAGCTGGACGGCGACGTAAA
CGGCCACAAGTTCAGCGTGTCCGGCGAGGGTGAGGGCGATGCCACCTACGGCAAGCTGACCCTGAAGTTC
ATCTGCACCACCGGCAAGCTGCCCGTGCCCTGGCCCACCCTCGTGACCACCCTGACCTACGGCGTGCAGT
GCTTCAGCCGCTACCCCGACCACATGAAGCAGCACGACTTCTTCAAGTCCGCCATGCCCGAAGGCTACAT
CCAGGAGCGCACCATCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAACATCCTGGGGCACA
AGCTGGAGTACAACGGTGCTGAGGTGACAAGACGGAGAGGGGTAGGACAATATTTGCTCGAGGAGGTCCT
TAGAAATAACCCCGGAGTAAGTTGCTGGTGGATGGCGGACGCTGGAGTTGAGGATCGCGGAGTCATGACG
GCTTTCATGCAGGCCCTTGGGTTCACCGCACAGCAAGGGGGCTGGGAGAAGTGCTAA
cpGFP with MLS Nucleic Acid Sequence
SEQ ID NO: 88
ATGGTGCTGGCCACCCGCGTGTTCAGCCTGGTGGGCAAGCGCGCCATCAGCACCAGCGTGTGCGTGCGCG
CCCACACCGGTGACTACAAGGATGACGATGACAAGCTCGATGGAGGATACCCATACGATGTTCCAGATTA
CGCTGCTCGAGGTTATCAAACAAGTTTGTACAAAAAAGCAGGCTCCACCATGGGGCATAACGTCTATATC
AAGGCCGACAAGCAGAAGAACGGCATCAAGGCGAACTTCAAGATCCGCCACAACATCGAGGACGGCGGCG
TGCAGCTCGCCTACCACTACCAGCAGAACACCCCCATCGGCGACGGCCCCGTGCTGCTGCCCGACAACCA
CTACCTGAGCgtgCAGTCCAAACTTTCGAAAGACCCCAACGAGAAGCGCGATCACATGGTCCTGCTGGAG
TTCGTGACCGCCGCCGGGATCACTCTCGGCATGGACGAGCTGTACAAGGGCGGTACCGGAGGGAGCATGG
TGAGCAAGGGCGAGGAGCTGTTCACCGGGGTGGTGCCCATCCTGGTCGAGCTGGACGGCGACGTAAACGG
CCACAAGTTCAGCGTGTCCGGCGAGGGTGAGGGCGATGCCACCTACGGCAAGCTGACCCTGAAGTTCATC
TGCACCACCGGCAAGCTGCCCGTGCCCTGGCCCACCCTCGTGACCACCCTGACCTACGGCGTGCAGTGCT
TCAGCCGCTACCCCGACCACATGAAGCAGCACGACTTCTTCAAGTCCGCCATGCCCGAAGGCTACATCCA
GGAGCGCACCATCTTCTTCAAGGACGACGGCAACTACAAGACCCGCGCCGAGGTGAAGTTCGAGGGCGAC
ACCCTGGTGAACCGCATCGAGCTGAAGGGCATCGACTTCAAGGAGGACGGCAACATCCTGGGGCACAAGC
TGGAGTACAACTAA

1. A recombinant acetyl-coenzyme A (acetyl-CoA) biosensor polypeptide comprising:
an acetyl-CoA binding protein having an amino acid sequence at least 95% identical to SEQ ID NO: 1, wherein the acetyl-CoA binding protein is divided into:
a first acetyl-CoA binding protein fragment comprising an N-terminal portion of the acetyl-CoA binding protein; and
a second acetyl-CoA binding protein fragment comprising a C-terminal portion of the acetyl-CoA binding protein;
wherein the first and second acetyl-CoA binding protein fragments collectively include all of the amino acids of the acetyl-CoA binding protein; and
a fluorescent protein inserted between the first and second acetyl-CoA binding protein fragments and attached to a C-terminus of the first acetyl-CoA binding protein fragment and an N-terminus of the second acetyl-CoA binding protein fragment; and
wherein:
(i) the C-terminus is an arginine at position 69 of SEQ ID NO: 1 (Arg69) and the N-terminus is a glutamic acid at position 70 of SEQ ID NO: 1 (Glu70);
(ii) the C-terminus is a tryptophan at position 23 of SEQ ID NO: 1 (Trp23) and the N-terminus is a proline at position 24 of SEQ ID NO: 1 (Pro24);
(iii) the C-terminus is a valine at position 71 of SEQ ID NO: 1 (Val71) and the N-terminus is a threonine at position 72 of SEQ ID NO: 1 (Thr72);
(iv) the C-terminus is an aspartic acid at position 99 of SEQ ID NO: 1 (Asp99) and the N-terminus is an alanine at position 100 of SEQ ID NO: 1 (Ala100);
(v) the C-terminus is an aspartic acid at 104 of SEQ ID NO: 1 (Asp104) and the N-terminus is an arginine at position 105 of SEQ ID NO: 1 (Arg105); or
(vi) the C-terminus is a glycine at position 116 of SEQ ID NO: 1 (Gly116) and the N-terminus is a phenylalanine at position 117 of SEQ ID NO: 1 (Phe117); and
wherein the recombinant acetyl-CoA biosensor polypeptide selectively binds acetyl-CoA, and the binding of acetyl-CoA induces a change in the fluorescence of the fluorescent protein.

2. The recombinant acetyl-CoA biosensor polypeptide of claim 1, wherein the fluorescent protein is a circularly permuted GFP (cpGFP), a circularly permuted yellow fluorescent protein (cpYFP), or a circularly permuted blue fluorescent protein (cpBFP).

3. The recombinant acetyl-CoA biosensor polypeptide of claim 2, wherein the cpGFP comprises an amino acid sequence of SEQ ID NO: 2, the cpYFP comprises an amino acid sequence of SEQ ID NO: 3, and the cpBFP comprises an amino acid sequence of SEQ ID NO: 4.

4. The recombinant acetyl-CoA biosensor polypeptide of claim 1, wherein the acetyl-CoA binding protein comprises an amino acid sequence at least 99% identical to SEQ ID NO: 1.

5. The recombinant acetyl-CoA biosensor polypeptide of claim 1, wherein the acetyl-CoA binding protein comprises the amino acid sequence of SEQ ID NO: 1.

6. The recombinant acetyl-CoA biosensor polypeptide of claim 1, wherein:
the fluorescent protein is either directly attached to the C-terminus of the first acetyl-CoA binding protein fragment or is attached by a first amino acid linker that is from 1 to 3 amino acids in length; and
the fluorescent protein is either directly attached to the N-terminus of the second acetyl-CoA binding protein fragment or is attached by a second linker that is from 1 to 3 amino acids in length.

7. The recombinant acetyl-CoA biosensor polypeptide of claim 6, wherein the first and second amino acid linkers are each independently selected from the group consisting of a Gly, Gly-Ala, Ala-Ser, and Gly-Ala-Ser.

8. The recombinant acetyl-CoA biosensor polypeptide of claim 6, wherein:
(i) the first linker is Gly-Ala and the second linker is Gly-Ala;
(ii) the first linker is Ala-Ser and the second linker is Ala-Ser;
(iii) the first linker is Gly-Ala-Ser and the second linker is Gly;
(iv) the C-terminus and N-terminus are directly attached to the fluorescent protein;
(v) the C-terminus is directly attached to the fluorescent protein and the second linker is Gly-Ala-Ser;
(vi) the first linker is Gly-Ala-Ser and the N-terminus is directly attached to the fluorescent protein;
(vii) the first linker is Gly-Ala and the N-terminus is directly attached to the fluorescent protein; or
(viii) the first linker is Gly and the second linker is Gly-Ala-Ser.

9. The recombinant acetyl-CoA biosensor polypeptide of claim 1, further comprising one or more of a histidine tag, a TEV cleavage site, a FLAG® tag, a human influenza hemagglutinin (HA) tag, a nuclear export signal, a nuclear localization signal, a cytoplasmic localization signal, and a mitochondrial localization signal at the N-terminal portion of the acetyl-CoA binding protein.

10. The recombinant acetyl-CoA biosensor polypeptide of claim 1, wherein:
(i) the recombinant acetyl-CoA biosensor polypeptide comprises the amino acid sequence of SEQ ID NO: 5;
(ii) the recombinant acetyl-CoA biosensor polypeptide comprises the amino acid sequence of SEQ ID NO: 6;
(iii) the recombinant acetyl-CoA biosensor polypeptide comprises the amino acid sequence of SEQ ID NO: 7;
(iv) the recombinant acetyl-CoA biosensor polypeptide comprises the amino acid sequence of SEQ ID NO: 8;
(v) the recombinant acetyl-CoA biosensor polypeptide comprises the amino acid sequence of SEQ ID NO: 9;
(vi) the recombinant acetyl-CoA biosensor polypeptide comprises the amino acid sequence of SEQ ID NO: 10;
(vii) the recombinant acetyl-CoA biosensor polypeptide comprises the amino acid sequence of SEQ ID NO: 11;
(viii) the recombinant acetyl-CoA biosensor polypeptide comprises the amino acid sequence of SEQ ID NO: 12;
(ix) the recombinant acetyl-CoA biosensor polypeptide comprises the amino acid sequence of SEQ ID NO: 13;
(x) the recombinant acetyl-CoA biosensor polypeptide comprises the amino acid sequence of SEQ ID NO: 14;
(xi) the recombinant acetyl-CoA biosensor polypeptide comprises the amino acid sequence of SEQ ID NO: 15;
(xii) the recombinant acetyl-CoA biosensor polypeptide comprises the amino acid sequence of SEQ ID NO: 16;
(xiii) the recombinant acetyl-CoA biosensor polypeptide comprises the amino acid sequence of SEQ ID NO: 17;
(xiv) the recombinant acetyl-CoA biosensor polypeptide comprises the amino acid sequence of SEQ ID NO: 18; or
(xv) the recombinant acetyl-CoA biosensor polypeptide comprises the amino acid sequence of SEQ ID NO: 19.

11. The recombinant acetyl-CoA biosensor polypeptide of claim 10, wherein the recombinant acetyl-CoA biosensor polypeptide comprises the amino acid of SEQ ID NO: 5.

12. An expression vector comprising:
a nucleic acid that encodes the recombinant acetyl-CoA biosensor polypeptide of claim 1; and
a promoter operably linked to the nucleic acid.

13. The expression vector of claim 12, wherein the expression vector is a lentiviral vector, an adeno-associated virus (AAV) vector, or a cytomegalovirus (CMV) vector.

14. A method of detecting acetyl-CoA in a sample comprising:
contacting the sample with the recombinant acetyl-CoA biosensor polypeptide of claim 1;
exciting the recombinant acetyl-CoA biosensor polypeptide in the sample at an excitation wavelength;
measuring a fluorescence intensity of the recombinant acetyl-CoA biosensor polypeptide in the sample at an emission wavelength; and
comparing the fluorescence intensity to a standard curve, wherein the fluorescence intensity correlates with a concentration of acetyl-CoA in the sample.

15. The method of claim 14, wherein the excitation wavelength is from about 460 nm to about 490 nm.

16. The method of claim 14, wherein the excitation wavelength is 485 nm.

17. The method of claim 14, wherein the emission wavelength is from about 513 nm to about 540 nm.

18. The method of claim 14, wherein the emission wavelength is 514 nm.

19. The method of claim 14, wherein the pH of the sample is maintained at a pH of 6.5-8.0.

20. A method of monitoring acetyl-CoA activity in a cell, comprising:
providing a cell with the recombinant acetyl-CoA biosensor polypeptide of claim 1;
exciting the recombinant acetyl-CoA biosensor polypeptide in the cell at a first excitation wavelength between about 400 nm and about 430 nm while measuring a first fluorescence intensity at an emission wavelength between about 513 nm and about 540 nm;
exciting the recombinant acetyl-CoA biosensor polypeptide in the cell at a second excitation wavelength between about 460 nm and about 490 nm while measuring a second fluorescence intensity at the emission wavelength; and
normalizing the second fluorescence intensity based on the first fluorescence intensity.

21. The method of claim 20, wherein normalizing comprises dividing the second fluorescence intensity by the first fluorescence intensity.

22. The method of claim 20, further comprising treating the cell with an acetyl-CoA precursor or nutrient affecting the function of the cell and comparing the normalized fluorescence intensity of the cell to the normalized fluorescence intensity of a control cell.

23. The method of claim 22, wherein one or more of a nuclear export signal, a nuclear localization signal, a cytoplasmic localization signal, and a mitochondrial localization signal is attached to an N-terminus of the recombinant acetyl-CoA biosensor polypeptide.

24. The method of claim 23, further comprising determining where acetyl-CoA is localized in the cell.

25. The method of claim 20, wherein the first excitation wavelength is 405 nm.

26. The method of claim 20, wherein the second excitation wavelength is 485 nm.

27. The method of claim 20, wherein the emission wavelength is 514 nm.

28. The method of claim 20, wherein the providing step comprises transforming the cell with a plasmid comprising a polynucleotide that encodes the recombinant acetyl-CoA biosensor polypeptide.


SEQUENCE LISTING (88 sequences)

SEQ ID NO: 1 - protein, 127 aa, synthetic construct
MKLTIIRLEKFSDQDRIDLQKIWPEYSPSSLQVDDNHRIYAARFNERLLAAVRVTLSGTEGALDSLRVREVTRRRGVGQYLLEEVLRNNPGVSCWWMADAGVEDRGVMTAFMQALGFTAQQGGWEKC

SEQ ID NO: 2 - protein, 241 aa, synthetic construct
NVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 3 - protein, 241 aa, synthetic construct
NVYIKADKQKNGIKANFKIRHNIEDGGVQLAYHYQQNTPIGDGPVLLPDNHYLSYQSVLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 4 - protein, 241 aa, synthetic construct
NVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLSHGVQCFSRYPDHMKQHDFFKSAMPEGYIQERTIFFKDDGNYKTRAEVKFEGDTLVNRIELKGIDFKEDGNILGHKLEYN

SEQ ID NO: 5 - protein, 372 aa, synthetic construct
MKLTIIRLEKFSDQDRIDLQKIWPEYSPSSLQVDDNHRIYAARFNERLLAAVRVTLSGTEGALDSLRVRGANVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGAEVTRRRGVGQYLLEEVLRNNPGVSCWWMADAGVEDRGVMTAFMQALGFTAQQGGWEKC

SEQ ID NO: 6 - protein, 372 aa, synthetic construct
MKLTIIRLEKFSDQDRIDLQKIWASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNASPEYSPSSLQVDDNHRIYAARFNERLLAAVRVTLSGTEGALDSLRVREVTRRRGVGQYLLEEVLRNNPGVSCWWMADAGVEDRGVMTAFMQALGFTAQQGGWEKC

SEQ ID NO: 7 - protein, 372 aa, synthetic construct
MKLTIIRLEKFSDQDRIDLQKIWPEYSPSSLQVDDNHRIYAARFNERLLAAVRVTLSGTEGALDSLRVREVASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNASTRRRGVGQYLLEEVLRNNPGVSCWWMADAGVEDRGVMTAFMQALGFTAQQGGWEKC

SEQ ID NO: 8 - protein, 372 aa, synthetic construct
MKLTIIRLEKFSDQDRIDLQKIWPEYSPSSLQVDDNHRIYAARFNERLLAAVRVTLSGTEGALDSLRVRASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNASEVTRRRGVGQYLLEEVLRNNPGVSCWWMADAGVEDRGVMTAFMQALGFTAQQGGWEKC

SEQ ID NO: 9 - protein, 372 aa, synthetic construct
MKLTIIRLEKFSDQDRIDLQKIWPEYSPSSLQVDDNHRIYAARFNERLLAAVRVTLSGTEGALDSLRVREVTRRRGVGQYLLEEVLRNNPGVSCWWMADASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNASAGVEDRGVMTAFMQALGFTAQQGGWEKC

SEQ ID NO: 10 - protein, 372 aa, synthetic construct
MKLTIIRLEKFSDQDRIDLQKIWPEYSPSSLQVDDNHRIYAARFNERLLAAVRVTLSGTEGALDSLRVREVTRRRGVGQYLLEEVLRNNPGVSCWWMADAGVEDASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNASRGVMTAFMQALGFTAQQGGWEKC

SEQ ID NO: 11 - protein, 372 aa, synthetic construct
MKLTIIRLEKFSDQDRIDLQKIWPEYSPSSLQVDDNHRIYAARFNERLLAAVRVTLSGTEGALDSLRVREVTRRRGVGQYLLEEVLRNNPGVSCWWMADAGVEDRGVMTAFMQALGASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNASFTAQQGGWEKC

SEQ ID NO: 12 - protein, 371 aa, synthetic construct
KLTIIRLEKFSDQDRIDLQKIWPEYSPSSLQVDDNHRIYAARFNERLLAAVRVTLSGTEGALDSLRVRGASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGEVTRRRGVGQYLLEEVLRNNPGVSCWWMADAGVEDRGVMTAFMQALGFTAQQGGWEKC

SEQ ID NO: 13 - protein, 371 aa, synthetic construct
MKLTIIRLEKFSDQDRIDLQKIWPEYSPSSLQVDDNHRIYAARFNERLLAAVRVTLSGTEGALDSLRVRGASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNEVTRRRGVGQYLLEEVLRNNPGVSCWWMADAGVEDRGVMTAFMQALGFTAQQGGWEKC

SEQ ID NO: 14 - protein, 370 aa, synthetic construct
MKLTIIRLEKFSDQDRIDLQKIWPEYSPSSLQVDDNHRIYAARFNERLLAAVRVTLSGTEGALDSLRVRGANVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNEVTRRRGVGQYLLEEVLRNNPGVSCWWMADAGVEDRGVMTAFMQALGFTAQQGGWEKC

SEQ ID NO: 15 - protein, 368 aa, synthetic construct
MKLTIIRLEKFSDQDRIDLQKIWPEYSPSSLQVDDNHRIYAARFNERLLAAVRVTLSGTEGALDSLRVRNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNEVTRRRGVGQYLLEEVLRNNPGVSCWWMADAGVEDRGVMTAFMQALGFTAQQGGWEKC

SEQ ID NO: 16 - protein, 372 aa, synthetic construct
MKLTIIRLEKFSDQDRIDLQKIWPEYSPSSLQVDDNHRIYAARFNERLLAAVRVTLSGTEGALDSLRVRGNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGASEVTRRRGVGQYLLEEVLRNNPGVSCWWMADAGVEDRGVMTAFMQALGFTAQQGGWEKC

SEQ ID NO: 17 - protein, 371 aa, synthetic construct
MKLTIIRLEKFSDQDRIDLQKIWPEYSPSSLQVDDNHRIYAARFNERLLAAVRVTLSGTEGALDSLRVRNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGASEVTRRRGVGQYLLEEVLRNNPGVSCWWMADAGVEDRGVMTAFMQALGFTAQQGGWEKC

SEQ ID NO: 18 - protein, 372 aa, synthetic construct
MKLTIIRLEKFSDQDRIDLQKIWPEYSPSSLQVDDNHRIYAARFNERLLAAVRVTLSGTEGALDSLRVRGANVYIKADKQKNGIKANFKIRHNIEDGGVQLAYHYQQNTPIGDGPVLLPDNHYLSYQSVLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGAEVTRRRGVGQYLLEEVLRNNPGVSCWWMADAGVEDRGVMTAFMQALGFTAQQGGWEKC

SEQ ID NO: 19 - protein, 372 aa, synthetic construct
MKLTIIRLEKFSDQDRIDLQKIWPEYSPSSLQVDDNHRIYAARFNERLLAAVRVTLSGTEGALDSLRVRGANVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLSHGVQCFSRYPDHMKQHDFFKSAMPEGYIQERTIFFKDDGNYKTRAEVKFEGDTLVNRIELKGIDFKEDGNILGHKLEYNGAEVTRRRGVGQYLLEEVLRNNPGVSCWWMADAGVEDRGVMTAFMQALGFTAQQGGWEKC

SEQ ID NO: 20 - DNA, 381 bp, synthetic construct
  source 1..381: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgagaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 21 - DNA, 723 bp, synthetic construct
  source 1..723: mol_type = other DNA
aacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaac

SEQ ID NO: 22 - DNA, 723 bp, synthetic construct
  source 1..723: mol_type = other DNA
aacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagctatcagtccgtactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaac

SEQ ID NO: 23 - DNA, 723 bp, synthetic construct
  source 1..723: mol_type = other DNA
aacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgtcccacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaac

SEQ ID NO: 24 - DNA, 1116 bp, synthetic construct
  source 1..1116: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgctgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 25 - DNA, 1116 bp, synthetic construct
  source 1..1116: mol_type = other DNA
atgaagctgacaatcattcgcctggagaaattttccgaccaggatagaatcgaccttcagaagatctgggcatctaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgcgtcacccgaatattctcccagtagcctccaagtagacgacaaccatcgaatctacgccgcgagatttaatgaacgactgcttgcggcggttagggtaaccctcagcggtacggaaggtgctctggactccctgcgggtccgagaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 26 - DNA, 1116 bp, synthetic construct
  source 1..1116: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgagaggtggcatctaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgcgtcaacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 27 - DNA, 1116 bp, synthetic construct
  source 1..1116: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgagcatctaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgcgtcagaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 28 - DNA, 1116 bp, synthetic construct
  source 1..1116: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgagaggtgacaagacggagaggggtaggacaatatttgctcgaggaggtccttagaaataaccccggagtaagttgctggtggatggcggacgcatctaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgcgtcagctggagttgaggatcgcggagtcatgacggctttcatgcaggcccttgggttcaccgcacagcaagggggctgggagaagtgc

SEQ ID NO: 29 - DNA, 1116 bp, synthetic construct
  source 1..1116: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgagaggtgacaagacggagaggggtaggacaatatttgctcgaggaggtccttagaaataaccccggagtaagttgctggtggatggcggacgctggagttgaggatgcatctaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgcgtcacgcggagtcatgacggctttcatgcaggcccttgggttcaccgcacagcaagggggctgggagaagtgc

SEQ ID NO: 30 - DNA, 1116 bp, synthetic construct
  source 1..1116: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgagaggtgacaagacggagaggggtaggacaatatttgctcgaggaggtccttagaaataaccccggagtaagttgctggtggatggcggacgctggagttgaggatcgcggagtcatgacggctttcatgcaggcccttggggcatctaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgcgtcattcaccgcacagcaagggggctgggagaagtgc

SEQ ID NO: 31 - DNA, 1116 bp, synthetic construct
  source 1..1116: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaagtaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 32 - DNA, 1113 bp, synthetic construct
  source 1..1113: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaagtaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 33 - DNA, 1110 bp, synthetic construct
  source 1..1110: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 34 - DNA, 1104 bp, synthetic construct
  source 1..1104: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 35 - DNA, 1116 bp, synthetic construct
  source 1..1116: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgcttcagaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 36 - DNA, 1113 bp, synthetic construct
  source 1..1113: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgcttcagaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 37 - DNA, 1116 bp, synthetic construct
  source 1..1116: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagctatcagtccgtactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgctgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 38 - DNA, 1116 bp, synthetic construct
  source 1..1116: mol_type = other DNA
atgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgtcccacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgctgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 39 - protein, 141 aa, synthetic construct
MHHHHHHENLYFQSMKLTIIRLEKFSDQDRIDLQKIWPEYSPSSLQVDDNHRIYAARFNERLLAAVRVTLSGTEGALDSLRVREVTRRRGVGQYLLEEVLRNNPGVSCWWMADAGVEDRGVMTAFMQALGFTAQQGGWEKC

SEQ ID NO: 40 - protein, 255 aa, synthetic construct
MHHHHHHENLYFQSNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 41 - protein, 255 aa, synthetic construct
MHHHHHHENLYFQSNVYIKADKQKNGIKANFKIRHNIEDGGVQLAYHYQQNTPIGDGPVLLPDNHYLSYQSVLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 42 - protein, 255 aa, synthetic construct
MHHHHHHENLYFQSNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLSHGVQCFSRYPDHMKQHDFFKSAMPEGYIQERTIFFKDDGNYKTRAEVKFEGDTLVNRIELKGIDFKEDGNILGHKLEYN

SEQ ID NO: 43 - protein, 386 aa, synthetic construct
MHHHHHHENLYFQSMKLTIIRLEKFSDQDRIDLQKIWPEYSPSSLQVDDNHRIYAARFNERLLAAVRVTLSGTEGALDSLRVRGANVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGAEVTRRRGVGQYLLEEVLRNNPGVSCWWMADAGVEDRGVMTAFMQALGFTAQQGGWEKC

SEQ ID NO: 44 - protein, 386 aa, synthetic construct
MHHHHHHENLYFQSMKLTIIRLEKFSDQDRIDLQKIWASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNASPEYSPSSLQVDDNHRIYAARFNERLLAAVRVTLSGTEGALDSLRVREVTRRRGVGQYLLEEVLRNNPGVSCWWMADAGVEDRGVMTAFMQALGFTAQQGGWEKC

SEQ ID NO: 45 - protein, 386 aa, synthetic construct
MHHHHHHENLYFQSMKLTIIRLEKFSDQDRIDLQKIWPEYSPSSLQVDDNHRIYAARFNERLLAAVRVTLSGTEGALDSLRVREVASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNASTRRRGVGQYLLEEVLRNNPGVSCWWMADAGVEDRGVMTAFMQALGFTAQQGGWEKC

SEQ ID NO: 46 - protein, 386 aa, synthetic construct
MHHHHHHENLYFQSMKLTIIRLEKFSDQDRIDLQKIWPEYSPSSLQVDDNHRIYAARFNERLLAAVRVTLSGTEGALDSLRVRASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNASEVTRRRGVGQYLLEEVLRNNPGVSCWWMADAGVEDRGVMTAFMQALGFTAQQGGWEKC

SEQ ID NO: 47 - protein, 386 aa, synthetic construct
MHHHHHHENLYFQSMKLTIIRLEKFSDQDRIDLQKIWPEYSPSSLQVDDNHRIYAARFNERLLAAVRVTLSGTEGALDSLRVREVTRRRGVGQYLLEEVLRNNPGVSCWWMADASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNASAGVEDRGVMTAFMQALGFTAQQGGWEKC

SEQ ID NO: 48 - protein, 386 aa, synthetic construct
MHHHHHHENLYFQSMKLTIIRLEKFSDQDRIDLQKIWPEYSPSSLQVDDNHRIYAARFNERLLAAVRVTLSGTEGALDSLRVREVTRRRGVGQYLLEEVLRNNPGVSCWWMADAGVEDASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNASRGVMTAFMQALGFTAQQGGWEKC

SEQ ID NO: 49 - protein, 386 aa, synthetic construct
MHHHHHHENLYFQSMKLTIIRLEKFSDQDRIDLQKIWPEYSPSSLQVDDNHRIYAARFNERLLAAVRVTLSGTEGALDSLRVREVTRRRGVGQYLLEEVLRNNPGVSCWWMADAGVEDRGVMTAFMQALGASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNASFTAQQGGWEKC

SEQ ID NO: 50 - protein, 371 aa, synthetic construct; identical to SEQ ID NO: 12

SEQ ID NO: 51 - protein, 385 aa, synthetic construct
MHHHHHHENLYFQSMKLTIIRLEKFSDQDRIDLQKIWPEYSPSSLQVDDNHRIYAARFNERLLAAVRVTLSGTEGALDSLRVRGASNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNEVTRRRGVGQYLLEEVLRNNPGVSCWWMADAGVEDRGVMTAFMQALGFTAQQGGWEKC

SEQ ID NO: 52 - protein, 384 aa, synthetic construct
MHHHHHHENLYFQSMKLTIIRLEKFSDQDRIDLQKIWPEYSPSSLQVDDNHRIYAARFNERLLAAVRVTLSGTEGALDSLRVRGANVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNEVTRRRGVGQYLLEEVLRNNPGVSCWWMADAGVEDRGVMTAFMQALGFTAQQGGWEKC

SEQ ID NO: 53 - protein, 382 aa, synthetic construct
MHHHHHHENLYFQSMKLTIIRLEKFSDQDRIDLQKIWPEYSPSSLQVDDNHRIYAARFNERLLAAVRVTLSGTEGALDSLRVRNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNEVTRRRGVGQYLLEEVLRNNPGVSCWWMADAGVEDRGVMTAFMQALGFTAQQGGWEKC

SEQ ID NO: 54 - protein, 386 aa, synthetic construct
MHHHHHHENLYFQSMKLTIIRLEKFSDQDRIDLQKIWPEYSPSSLQVDDNHRIYAARFNERLLAAVRVTLSGTEGALDSLRVRGNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGASEVTRRRGVGQYLLEEVLRNNPGVSCWWMADAGVEDRGVMTAFMQALGFTAQQGGWEKC

SEQ ID NO: 55 - protein, 385 aa, synthetic construct
MHHHHHHENLYFQSMKLTIIRLEKFSDQDRIDLQKIWPEYSPSSLQVDDNHRIYAARFNERLLAAVRVTLSGTEGALDSLRVRNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGASEVTRRRGVGQYLLEEVLRNNPGVSCWWMADAGVEDRGVMTAFMQALGFTAQQGGWEKC

SEQ ID NO: 56 - protein, 386 aa, synthetic construct
MHHHHHHENLYFQSMKLTIIRLEKFSDQDRIDLQKIWPEYSPSSLQVDDNHRIYAARFNERLLAAVRVTLSGTEGALDSLRVRGANVYIKADKQKNGIKANFKIRHNIEDGGVQLAYHYQQNTPIGDGPVLLPDNHYLSYQSVLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGAEVTRRRGVGQYLLEEVLRNNPGVSCWWMADAGVEDRGVMTAFMQALGFTAQQGGWEKC

SEQ ID NO: 57 - protein, 386 aa, synthetic construct
MHHHHHHENLYFQSMKLTIIRLEKFSDQDRIDLQKIWPEYSPSSLQVDDNHRIYAARFNERLLAAVRVTLSGTEGALDSLRVRGANVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLSHGVQCFSRYPDHMKQHDFFKSAMPEGYIQERTIFFKDDGNYKTRAEVKFEGDTLVNRIELKGIDFKEDGNILGHKLEYNGAEVTRRRGVGQYLLEEVLRNNPGVSCWWMADAGVEDRGVMTAFMQALGFTAQQGGWEKC

SEQ ID NO: 58 - DNA, 423 bp, synthetic construct
  source 1..423: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgagaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 59 - DNA, 765 bp, synthetic construct
  source 1..765: mol_type = other DNA
atgcatcaccatcaccatcacgaaaacctgtacttccaaagcaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaac

SEQ ID NO: 60 - DNA, 765 bp, synthetic construct
  source 1..765: mol_type = other DNA
atgcatcaccatcaccatcacgaaaacctgtacttccaaagcaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagctatcagtccgtactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaac

SEQ ID NO: 61 - DNA, 765 bp, synthetic construct
  source 1..765: mol_type = other DNA
atgcatcaccatcaccatcacgaaaacctgtacttccaaagcaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgtcccacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaac

SEQ ID NO: 62 - DNA, 1158 bp, synthetic construct
  source 1..1158: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgctgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 63 - DNA, 1158 bp, synthetic construct
  source 1..1158: mol_type = other DNA
atgcatcaccatcaccatcacgaaaacctgtacttccaaagcatgaagctgacaatcattcgcctggagaaattttccgaccaggatagaatcgaccttcagaagatctgggcatctaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgcgtcacccgaatattctcccagtagcctccaagtagacgacaaccatcgaatctacgccgcgagatttaatgaacgactgcttgcggcggttagggtaaccctcagcggtacggaaggtgctctggactccctgcgggtccgagaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 64 - DNA, 1158 bp, synthetic construct
  source 1..1158: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgagaggtggcatctaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgcgtcaacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 65 - DNA, 1158 bp, synthetic construct
  source 1..1158: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgagcatctaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgcgtcagaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 66 - DNA, 1158 bp, synthetic construct
  source 1..1158: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgagaggtgacaagacggagaggggtaggacaatatttgctcgaggaggtccttagaaataaccccggagtaagttgctggtggatggcggacgcatctaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgcgtcagctggagttgaggatcgcggagtcatgacggctttcatgcaggcccttgggttcaccgcacagcaagggggctgggagaagtgc

SEQ ID NO: 67 - DNA, 1158 bp, synthetic construct
  source 1..1158: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgagaggtgacaagacggagaggggtaggacaatatttgctcgaggaggtccttagaaataaccccggagtaagttgctggtggatggcggacgctggagttgaggatgcatctaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgcgtcacgcggagtcatgacggctttcatgcaggcccttgggttcaccgcacagcaagggggctgggagaagtgc

SEQ ID NO: 68 - DNA, 1158 bp, synthetic construct
  source 1..1158: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgagaggtgacaagacggagaggggtaggacaatatttgctcgaggaggtccttagaaataaccccggagtaagttgctggtggatggcggacgctggagttgaggatcgcggagtcatgacggctttcatgcaggcccttggggcatctaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgcgtcattcaccgcacagcaagggggctgggagaagtgc

SEQ ID NO: 69 - DNA, 1158 bp, synthetic construct
  source 1..1158: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaagtaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 70 - DNA, 1155 bp, synthetic construct
  source 1..1155: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaagtaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 71 - DNA, 1152 bp, synthetic construct
  source 1..1152: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 72 - DNA, 1146 bp, synthetic construct
  source 1..1146: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 73 - DNA, 1158 bp, synthetic construct
  source 1..1158: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgcttcagaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 74 - DNA, 1155 bp, synthetic construct
  source 1..1155: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgcttcagaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 75 - DNA, 1158 bp, synthetic construct
  source 1..1158: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagctatcagtccgtactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgctgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 76 - DNA, 1158 bp, synthetic construct
  source 1..1158: mol_type = other DNA
atgcatcaccatcaccatcacgaaaacctgtacttccaaagcatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgtcccacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgctgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgc

SEQ ID NO: 77 - protein, 422 aa, synthetic construct
MVTGPKKKRKVDYKDDDDKLDGGYPYDVPDYAARGYQTSLYKKAGSTMGHMKLTIIRLEKFSDQDRIDLQKIWPEYSPSSLQVDDNHRIYAARFNERLLAAVRVTLSGTEGALDSLRVRGANVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGAEVTRRRGVGQYLLEEVLRNNPGVSCWWMADAGVEDRGVMTAFMQALGFTAQQGGWEKC

SEQ ID NO: 78 - protein, 291 aa, synthetic construct
MVTGPKKKRKVDYKDDDDKLDGGYPYDVPDYAARGYQTSLYKKAGSTMGHNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 79 - protein, 426 aa, synthetic construct
MVTGLQKKLEELELDDYKDDDDKLDGGYPYDVPDYAARGYQTSLYKKAGSTMGHMKLTIIRLEKFSDQDRIDLQKIWPEYSPSSLQVDDNHRIYAARFNERLLAAVRVTLSGTEGALDSLRVRGANVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGAEVTRRRGVGQYLLEEVLRNNPGVSCWWMADAGVEDRGVMTAFMQALGFTAQQGGWEKC

SEQ ID NO: 80 - protein, 295 aa, synthetic construct
MVTGLQKKLEELELDDYKDDDDKLDGGYPYDVPDYAARGYQTSLYKKAGSTMGHNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 81 - protein, 438 aa, synthetic construct
MVLATRVFSLVGKRAISTSVCVRAHTGDYKDDDDKLDGGYPYDVPDYAARGYQTSLYKKAGSTMGHMKLTIIRLEKFSDQDRIDLQKIWPEYSPSSLQVDDNHRIYAARFNERLLAAVRVTLSGTEGALDSLRVRGANVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGAEVTRRRGVGQYLLEEVLRNNPGVSCWWMADAGVEDRGVMTAFMQALGFTAQQGGWEKC

SEQ ID NO: 82 - protein, 307 aa, synthetic construct
MVLATRVFSLVGKRAISTSVCVRAHTGDYKDDDDKLDGGYPYDVPDYAARGYQTSLYKKAGSTMGHNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 83 - DNA, 1269 bp, synthetic construct
  source 1..1269: mol_type = other DNA
atggtgaccggtccaaagaagaagcgtaaggtagactacaaggatgacgatgacaagctcgatggaggatacccatacgatgttccagattacgctgctcgaggttatcaaacaagtttgtacaaaaaagcaggctccaccatggggcatatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgctgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgctaa

SEQ ID NO: 84 - DNA, 876 bp, synthetic construct
  source 1..876: mol_type = other DNA
atggtgaccggtccaaagaagaagcgtaaggtagactacaaggatgacgatgacaagctcgatggaggatacccatacgatgttccagattacgctgctcgaggttatcaaacaagtttgtacaaaaaagcaggctccaccatggggcataacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactaa

SEQ ID NO: 85 - DNA, 1281 bp, synthetic construct
  source 1..1281: mol_type = other DNA
atggtgaccggtctgcagaaaaagctggaagagctggaactggacgactacaaggatgacgatgacaagctcgatggaggatacccatacgatgttccagattacgctgctcgaggttatcaaacaagtttgtacaaaaaagcaggctccaccatggggcatatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgctgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgctaa

SEQ ID NO: 86 - DNA, 888 bp, synthetic construct
  source 1..888: mol_type = other DNA
atggtgaccggtctgcagaaaaagctggaagagctggaactggacgactacaaggatgacgatgacaagctcgatggaggatacccatacgatgttccagattacgctgctcgaggttatcaaacaagtttgtacaaaaaagcaggctccaccatggggcataacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactaa

SEQ ID NO: 87 - DNA, 1317 bp, synthetic construct
  source 1..1317: mol_type = other DNA
atggtgctggccacccgcgtgttcagcctggtgggcaagcgcgccatcagcaccagcgtgtgcgtgcgcgcccacaccggtgactacaaggatgacgatgacaagctcgatggaggatacccatacgatgttccagattacgctgctcgaggttatcaaacaagtttgtacaaaaaagcaggctccaccatggggcatatgaagctgacaatcattcgcctggagaaattttccgaccaggatagaatcgaccttcagaagatctggcccgaatattctcccagtagcctccaagtagacgacaaccatcgaatctacgccgcgagatttaatgaacgactgcttgcggcggttagggtaaccctcagcggtacggaaggtgctctggactccctgcgggtccgaggagcaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacggtgctgaggtgacaagacggagaggggtaggacaatatttgctcgaggaggtccttagaaataaccccggagtaagttgctggtggatggcggacgctggagttgaggatcgcggagtcatgacggctttcatgcaggcccttgggttcaccgcacagcaagggggctgggagaagtgctaa

SEQ ID NO: 88 - DNA, 924 bp, synthetic construct
  source 1..924: mol_type = other DNA
atggtgctggccacccgcgtgttcagcctggtgggcaagcgcgccatcagcaccagcgtgtgcgtgcgcgcccacaccggtgactacaaggatgacgatgacaagctcgatggaggatacccatacgatgttccagattacgctgctcgaggttatcaaacaagtttgtacaaaaaagcaggctccaccatggggcataacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactaa